SEQUENCE LISTING (62 sequences)

SEQ ID NO: 1 - DNA, 20 bp, unidentified
  source 1..20: mol_type = unassigned DNA
ctcccactcttccaccttcg

SEQ ID NO: 2 - DNA, 20 bp, unidentified
  source 1..20: mol_type = unassigned DNA
gcctctcttgctcagtgtcc

SEQ ID NO: 3 - DNA, 24 bp, unidentified
  source 1..24: mol_type = unassigned DNA
caacaattcctggcgttaccttgg

SEQ ID NO: 4 - DNA, 24 bp, unidentified
  source 1..24: mol_type = unassigned DNA
gaaagccctgtattccgtctcctt

SEQ ID NO: 5 - DNA, 20 bp, unidentified
  source 1..20: mol_type = unassigned DNA
tgaattccctgggtgagaag

SEQ ID NO: 6 - DNA, 21 bp, unidentified
  source 1..21: mol_type = unassigned DNA
tcactcttcacctgctccact

SEQ ID NO: 7 - DNA, 21 bp, unidentified
  source 1..21: mol_type = unassigned DNA
gggtcattcagaggcacactg

SEQ ID NO: 8 - DNA, 20 bp, unidentified
  source 1..20: mol_type = unassigned DNA
ctggctgtcctgtcaaggct

SEQ ID NO: 9 - DNA, 20 bp, unidentified
  source 1..20: mol_type = unassigned DNA
gttagtgtaccgcaccctcc

SEQ ID NO: 10 - DNA, 21 bp, unidentified
  source 1..21: mol_type = unassigned DNA
aaacacagactgacccttccc

SEQ ID NO: 11 - DNA, 21 bp, unidentified
  source 1..21: mol_type = unassigned DNA
tgcctatgtctcagcctcttc

SEQ ID NO: 12 - DNA, 20 bp, unidentified
  source 1..20: mol_type = unassigned DNA
gaggccatttgggaacttct

SEQ ID NO: 13 - DNA, 20 bp, unidentified
  source 1..20: mol_type = unassigned DNA
gttctctgggaaatcgtgga

SEQ ID NO: 14 - DNA, 20 bp, unidentified
  source 1..20: mol_type = unassigned DNA
ggaaattggggtaggaagga

SEQ ID NO: 15 - DNA, 20 bp, unidentified
  source 1..20: mol_type = unassigned DNA
aaatacctgtggccttgggc

SEQ ID NO: 16 - DNA, 21 bp, unidentified
  source 1..21: mol_type = unassigned DNA
cttgggatccacactctccag

SEQ ID NO: 17 - DNA, 20 bp, unidentified
  source 1..20: mol_type = unassigned DNA
tgtgttccaccaggagatgt

SEQ ID NO: 18 - DNA, 19 bp, unidentified
  source 1..19: mol_type = unassigned DNA
aggtgagctgaacgaggaa

SEQ ID NO: 19 - DNA, 20 bp, unidentified
  source 1..20: mol_type = unassigned DNA
gcatcctgggctacactgag

SEQ ID NO: 20 - DNA, 20 bp, unidentified
  source 1..20: mol_type = unassigned DNA
gtcaaaggtggaggagtggg

SEQ ID NO: 21 - DNA, 20 bp, unidentified
  source 1..20: mol_type = unassigned DNA
acaaatgaatggggaaagac

SEQ ID NO: 22 - DNA, 20 bp, unidentified
  source 1..20: mol_type = unassigned DNA
ttatttgaaacagactgggc

SEQ ID NO: 23 - DNA, 19 bp, unidentified
  source 1..19: mol_type = unassigned DNA
ttctttcctcttccccttg

SEQ ID NO: 24 - DNA, 21 bp, unidentified
  source 1..21: mol_type = unassigned DNA
cctcttctggagtttatttgg

SEQ ID NO: 25 - DNA, 20 bp, unidentified
  source 1..20: mol_type = unassigned DNA
gcagtatttacaacagaggg

SEQ ID NO: 26 - DNA, 20 bp, unidentified
  source 1..20: mol_type = unassigned DNA
tcccaaaagaagttttgctg

SEQ ID NO: 27 - DNA, 20 bp, unidentified
  source 1..20: mol_type = unassigned DNA
gaccaaggaaaactcactac

SEQ ID NO: 28 - DNA, 20 bp, unidentified
  source 1..20: mol_type = unassigned DNA
ctgtttaactggtatggcac

SEQ ID NO: 29 - DNA, 20 bp, unidentified
  source 1..20: mol_type = unassigned DNA
atgtggagtatgagagtgac

SEQ ID NO: 30 - DNA, 20 bp, unidentified
  source 1..20: mol_type = unassigned DNA
cttgtatctcggtttgaagc

SEQ ID NO: 31 - DNA, 18 bp, unidentified
  source 1..18: mol_type = unassigned DNA
tggtccacttcatcaacc

SEQ ID NO: 32 - DNA, 21 bp, unidentified
  source 1..21: mol_type = unassigned DNA
ttctgtatttcttcaggagac

SEQ ID NO: 33 - DNA, 20 bp, unidentified
  source 1..20: mol_type = unassigned DNA
ggagacttcaaatgaaggag

SEQ ID NO: 34 - DNA, 21 bp, unidentified
  source 1..21: mol_type = unassigned DNA
cagaaagtgtggtattctcag

SEQ ID NO: 35 - DNA, 20 bp, unidentified
  source 1..20: mol_type = unassigned DNA
caccgagacaccatgagagc

SEQ ID NO: 36 - DNA, 21 bp, unidentified
  source 1..21: mol_type = unassigned DNA
ctgcttggacacaaaggctgc

SEQ ID NO: 37 - DNA, 19 bp, unidentified
  source 1..19: mol_type = unassigned DNA
tctgggagggcttggttgt

SEQ ID NO: 38 - DNA, 20 bp, unidentified
  source 1..20: mol_type = unassigned DNA
aactggtatggcacaggtga

SEQ ID NO: 39 - DNA, 20 bp, unidentified
  source 1..20: mol_type = unassigned DNA
gcatcctgggctacactgag

SEQ ID NO: 40 - DNA, 20 bp, unidentified
  source 1..20: mol_type = unassigned DNA
gtcaaaggtggaggagtggg

SEQ ID NO: 41 - DNA, 20 bp, unidentified
  source 1..20: mol_type = unassigned DNA
agccttgtttgtactgtatg

SEQ ID NO: 42 - DNA, 20 bp, unidentified
  source 1..20: mol_type = unassigned DNA
aaaatggtggtttagcagag

SEQ ID NO: 43 - DNA, 21 bp, unidentified
  source 1..21: mol_type = unassigned DNA
aaagaaagccaacactaaacc

SEQ ID NO: 44 - DNA, 19 bp, unidentified
  source 1..19: mol_type = unassigned DNA
tggtcatttcgttaaaggc

SEQ ID NO: 45 - DNA, 20 bp, unidentified
  source 1..20: mol_type = unassigned DNA
tcaatgacatttcacacacg

SEQ ID NO: 46 - DNA, 20 bp, unidentified
  source 1..20: mol_type = unassigned DNA
tccatcttggataaggtcag

SEQ ID NO: 47 - DNA, 20 bp, unidentified
  source 1..20: mol_type = unassigned DNA
caatacagatggcttcaagg

SEQ ID NO: 48 - DNA, 22 bp, unidentified
  source 1..22: mol_type = unassigned DNA
gatgtattcaaatgactcaggg

SEQ ID NO: 49 - DNA, 20 bp, unidentified
  source 1..20: mol_type = unassigned DNA
caagagcaccataaccttag

SEQ ID NO: 50 - DNA, 21 bp, unidentified
  source 1..21: mol_type = unassigned DNA
ctcgttttctctttatggtgg

SEQ ID NO: 51 - DNA, 20 bp, unidentified
  source 1..20: mol_type = unassigned DNA
aatctgggttttgtgtcttc

SEQ ID NO: 52 - DNA, 20 bp, unidentified
  source 1..20: mol_type = unassigned DNA
aaaagttgtgtaccttgtgg

SEQ ID NO: 53 - DNA, 20 bp, unidentified
  source 1..20: mol_type = unassigned DNA
aaatccaagttcactgatcc

SEQ ID NO: 54 - DNA, 20 bp, unidentified
  source 1..20: mol_type = unassigned DNA
cctctttcttatagcacagc

SEQ ID NO: 55 - DNA, 21 bp, unidentified
  source 1..21: mol_type = unassigned DNA
atagatgttaccctgtgtgag

SEQ ID NO: 56 - DNA, 20 bp, unidentified
  source 1..20: mol_type = unassigned DNA
aagagactaagccagttagg

SEQ ID NO: 57 - DNA, 20 bp, unidentified
  source 1..20: mol_type = unassigned DNA
ggtcttattggtcctaaggg

SEQ ID NO: 58 - DNA, 21 bp, unidentified
  source 1..21: mol_type = unassigned DNA
gtagaagatcttggtaaagcg

SEQ ID NO: 59 - DNA, 21 bp, unidentified
  source 1..21: mol_type = unassigned DNA
acacagaggtagatattggag

SEQ ID NO: 60 - DNA, 20 bp, unidentified
  source 1..20: mol_type = unassigned DNA
ctttttctgagtctctcctg

SEQ ID NO: 61 - DNA, 20 bp, unidentified
  source 1..20: mol_type = unassigned DNA
ccctgaaaagattgcttctg

SEQ ID NO: 62 - DNA, 18 bp, unidentified
  source 1..18: mol_type = unassigned DNA
aggaccttgtctgaagtg

(19) United States
(12) Patent Application Publication
(10) Pub. No.: US 2026/0355234 A1
(43) Pub. Date: Nov. 12, 2026

(54) MICROBIOME-CERIA NANOCOMPOSITIONS AND METHODS FOR USE THEREOF

(71) Applicant: University of Central Florida Research Foundation, Inc., Orlando, FL (US)

(72) Inventors: Melanie Coathup, Orlando, FL (US); Sudipta Seal, Orlando, FL (US); Abinaya Sindu Pugazhendhi, Orlando, FL (US)

(73) Assignee: University of Central Florida Research Foundation, Inc., Orlando, FL (US)

(21) Appl. No.: 19/434,105

(22) Filed: Dec. 29, 2025

Related U.S. Application Data
(60) Provisional application No. 63/740,631, filed on Dec. 31, 2024.

(51) Int. Cl. A61K 33/24; A61K 9/16; A61P 19/02

(52) U.S. Cl. CPC A61K 33/24; A61K 9/167; A61P 19/02

(57) ABSTRACT

Microbiome-ceria nanocompositions and methods for application of these nanocompositions to benefit human health. The nanocomposition formulations include cerium oxide nanoparticles combined with proteins and/or other bioactive agents isolated from commensal microbes such as Lactobacillus acidophilus (L. acidophilus).

### FIELD OF THE INVENTION

The invention generally relates to bioactive agents released or isolated from a microbiome and their potential for application in human health; particularly to nanocompositions formed by combination of therapeutic nanoparticles with bioactive agents released/isolated from a microbiome; and most particularly to nanocompositions including cerium oxide nanoparticles and proteins isolated from commensal microbes such as Lactobacillus acidophilus (L. acidophilus).

### SEQUENCE LISTING INCORPORATION BY REFERENCE

The Sequence Listing in XML format titled “UCF-017utilitySEQLIST.xml”, created on Dec. 2, 2025, and having a file size of 55,264 bytes, is incorporated herein by reference in its entirety pursuant to 37 C.F.R. §§ 1.834(c)(1) and 1.835(a)(2). The Sequence Listing XML file complies with WIPO Standard ST.26.

### BACKGROUND

Humans have co-evolved with a microbiome, a microbial community that inhabits and interacts with the human body. While a variety of organisms, such as fungi and viruses, exist within the microbiome, most of the organisms (within the microbiome) are commensal bacteria which reside on or within the body without harming human health. Microbiomes play critical roles in human health and can be found in the oral cavity, in the gut, and/or on the skin. For example, microbiomes stimulate the immune and metabolic systems, break down potentially toxic food compounds, and synthesize vitamins and amino acids. The microbiota in a healthy human also provides protection from pathogenic organisms that enter and/or interact with the body. For example, the gut microbiota hosts ~100 trillion beneficial/probiotic/commensal microbes of ~1000 species and 28 different phyla. In turn, this microbiota directly regulates many aspects of physiology (immune system, metabolism, nutrient storage, blood pressure, and/or destruction of harmful bacteria) and can be utilized in treatment of heart disease, cancer, diabetes, and/or various inflammatory diseases, including those related to brain and bone health. Although known effects are demonstrated by interaction, the mechanisms of interaction between the microbes and the host cells and tissues remain elusive. As such, strategies that improve understanding of commensal microbes and their role in the body are needed. Further, leveraging the broad-spectrum protection they can potentially deliver through engineering microbiome-based therapeutic strategies may provide a novel approach capable of preventing and/or mitigating many human health challenges, for example treatment of periprosthetic joint infection (PJI) that can occur following joint replacement surgery.

### SUMMARY OF THE INVENTION

The instant invention provides this needed therapeutic strategy in a probiotic microbiome-ceria nanoparticle formulation. This formulation is capable of functioning as both an active antibacterial agent and as a pro-tissue healing agent for maintaining health as a countermeasure to disease, trauma, infection, and/or aging.

In development of the formulations, the instant inventors isolated proteins (both secreted and internal proteins) released from the gut probiotic commensal bacteria, Lactobacillus acidophilus (L. acidophilus). L. acidophilus qualifies for presumption of safety status and are thus considered safe by the U.S. Food and Drug Administration. The isolated extracellular-derived L. acidophilus proteins are abbreviated as LaEPs.

Tissue damage and subsequent repair involve multiple cellular events and tissue cascades. The LaEPs act on multiple events and tissue cascades and (the LaEPs) have been shown to upregulate cell metabolism, to promote multiple types of new tissue formation, to control and downregulate a harmful inflammatory response, and to change cells from releasing harmful damaging products to releasing pro-repair products instead. At the same dose, the LaEPs beneficially activate certain immune cells towards activity that promotes the removal of harmful bacteria and cancer cells.

In development of the formulations, the instant inventors next considered therapeutic nanoparticles. Nanoscale cerium oxide (also referred to interchangeably herein as nanoceria, ceria, nanozymes, and cerium oxide nanoparticles (CNPs)) is an artificial enzyme capable of functioning as an antioxidant having demonstrated biomedical utility. Previous research conducted by the instant inventors indicated that ceria nanoparticles can regulate harmful inflammation that causes tissue damage and loss, while at the same time, also promote other cells to form new healthy tissue. Cerium-oxide nanoparticles coated with the secreted and internal proteins of L. acidophilus were found to exhibit a synergistic therapeutic host cell response. It is anticipated that these protein-coated ceria nanoparticles will reduce growth and viability of pathogenic bacteria, thereby reduce infection, while also regulating inflammation and boosting healthy tissue repair.

Subsequent to the CNP-LaEPs research, further research conducted by the instant inventors has shown that microbiome proteins reduce pathogenic growth, beneficially regulate inflammation, and promote bone tissue repair. This concept can be theoretically expanded to multiple types of ceria nanoparticles, as differing formulations of ceria nanoparticles (i.e., changes in the Ce³⁺ and Ce⁴⁺ ratio) may bind different proteins. Such concept presents the possibility of coating ceria nanoparticles with proteins (or other biomolecules and/or bioactive agents) from a vast array of differing probiotics found within the gut microbiome or within other microbiomes. Thus, in one dose, the bioactive ingredients from several different probiotics together with synergistic ceria activity, can be administered as a multi-functional therapy. Therefore, the broad and beneficial pro-healing effects of probiotic bacteria in the gut, could be delivered directly to other areas of the body where improved tissue healing, and repair is needed, such as in areas in which infection prevention is needed. For example, the probiotics in the gut help to stop the pathogenic bacterial colonization of the intestinal wall. This blueprint could be applied to implants that are surgically inserted into the body and where pathogens race to its surface once implanted. By utilizing the probiotic-coated nanoparticles within a microbiome-nanoparticle-based strategy, the adhesion and growth of pathogenic bacteria onto the implant surface may be reduced, while at the same time, surrounding healthy tissue repair and regeneration is boosted.

The instant invention contemplates a new therapeutic concept; combination of nanoscale cerium oxide with bioactive agents extracted from the microbiome to provide nanocompositions potentially exhibiting both bacteriostatic and bactericidal activity. A non-limiting example is a nanocomposition in which a bioactive agent portion of the nanocomposition provides bacteriostatic activity and the nanoscale cerium oxide portion of the nanocomposition provides bactericidal activity.

Most therapies target only a single cell or tissue type. In an aspect, the instant invention provides a new therapeutic concept using nanoscale cerium oxide in combination with bioactive agents released or isolated from a microbiome, such as but not limited to probiotic peptides and/or proteins. By combining the use of both cerium oxide nanoparticles and probiotic peptides/proteins in a single therapeutic composition, the invention provides a multidimensional approach to tissue repair.

In a general aspect, the instant invention contemplates deliberate alteration of microbiomes as therapy. In a basic aspect, the invention provides a composition and a method for simultaneously reducing pathogenic bacteria and boosting healthy tissue. In another basic aspect, the invention provides a new therapeutic composition capable of preventing pathogenic bacteria from colonization.

In a general embodiment, the invention provides a nanocomposition including nanoparticles and at least one bioactive agent released or isolated from a microbiome. The nanoparticles can be therapeutic nanoparticles. In some embodiments, the at least one bioactive agent is coated onto surfaces of the nanoparticles or is unbound to the nanoparticles. In other embodiments, the nanocomposition includes both a bioactive agent coated onto surfaces of the nanoparticles and a bioactive agent unbound to the nanoparticles. The bioactive agent unbound to the nanoparticles can be the same bioactive agent as that coating the surfaces of the nanoparticles.

In an embodiment, the invention provides a composition including therapeutic nanoparticles and at least one bioactive agent released or isolated from a microbiome. The composition can be referred to as a nanocomposition as it includes nanoscale elements and/or ingredients. Therapeutic nanoparticles provide a beneficial or desired property and/or effect. A bioactive agent refers to a substance that affects a living organism, preferably in a beneficial way. Non-limiting examples of bioactive agents are proteins, peptides, carbohydrates, lipids, and nucleic acids. In the formulation of the composition, the bioactive agent can be coated onto surfaces of the therapeutic nanoparticles. The bioactive agent can also be unbound to the therapeutic nanoparticles. In a preferred embodiment, the composition/nanocomposition includes both unbound bioactive agent and bioactive agent coated onto surfaces of the therapeutic nanoparticles. The unbound bioactive agent can be the same bioactive agent that is coated onto surfaces of the therapeutic nanoparticles but the use of different and/or multiple bioactive agents is also contemplated. In preferred embodiments of the invention, the bound and unbound bioactive agents are the same and/or identical bioactive agent.

In an aspect, the invention provides a composition comprising cerium oxide nanoparticles (CNPs) and at least one bioactive agent released by or isolated from probiotic organisms. A probiotic organism can be any organism isolated from a microbiome. Preferred probiotic organisms are commensal microbes, such as, but not limited to, bacteria. A non-limiting example is Lactobacillus acidophilus (L. acidophilus).

In another aspect, the invention provides a composition comprising cerium oxide nanoparticles (CNPs) and at least one peptide or protein released by or isolated from probiotic organisms. In a non-limiting example, the peptide or protein can be an intracellular peptide/protein isolated from the probiotic organisms, or a secreted/extracellular protein released by the probiotic organisms. In one embodiment, the protein is an extracellular protein isolated from Lactobacillus acidophilus (L. acidophilus). Extracellular proteins isolated from Lactobacillus acidophilus are referred to herein as LaEPs (Lactobacillus acidophilus extracellular proteins). Various, non-limiting examples of LaEPs are listed in Table 1.

The surfaces of cerium oxide nanoparticles exhibit varying levels of Ce³⁺ surface sites and Ce⁴⁺ surface sites. Changing the composition of surface sites changes surface activity allowing attachment/detachment of a variety of different bioactive agents. At reactive surface sites, cerium ions have the ability to easily undergo redox recycling and thus, are capable of drastically adjusting their electronic configurations and versatile catalytic activities. Wei et al. have shown that an engineered artificial nanozyme composed of cerium oxide and designed to possess a higher fraction of trivalent (Ce³⁺) surface sites (relative to Ce⁴⁺), mitigates ionizing radiation-induced loss in bone area, bone architecture, and strength⁴¹.

In an embodiment, the cerium oxide nanoparticles have at least one of Ce³⁺ surface sites and Ce⁴⁺ surface sites. In a preferred, although non-limiting embodiment, the cerium oxide nanoparticles include both Ce³⁺ surface sites and Ce⁴⁺ surface sites.

In another embodiment, the cerium oxide nanoparticles have increased levels of Ce³⁺ surface sites relative to levels of Ce⁴⁺ surface sites, such as, but not limited to cerium oxide CeONP^{60/40} and cerium oxide CeONP^{70/30}.

In another aspect, the described composition/nanocomposition is a pharmaceutical composition formulated for administration as a therapeutic solution, a gel, a paste, or a topical cream. Such pharmaceutical compositions can be used as additives and formulated with other compositions, such as, but not limited to, therapeutic compositions, preventive compositions, and health/wellness compositions.

In another embodiment, the invention provides an implant configured for insertion into a body (a surgically placed/inserted implant), such as, but not limited to, a joint replacement implant. The implant is coated with the inventive pharmaceutical composition such that infection after implantation (into the body) can be prevented and/or treated. The body is preferably a human body but is contemplated as the body of any animal that can benefit from implants.

In another aspect, the components of the inventive compositions, nanocompositions, pharmaceutical compositions, and/or implants can be packaged in containers and assembled in kits together with instructions for use.

In another aspect, the inventive compositions, nanocompositions, pharmaceutical compositions, and/or implants can be packaged in containers together with instructions for use.

In another embodiment, the invention provides a kit comprising an implant for insertion into a body, preferably surgical insertion. The implant of the kit has a surface at least partially coated with at least one of the inventive compositions, nanocompositions, and pharmaceutical compositions. In a preferred, albeit non-limiting embodiment, the surface of the implant is completely coated with the inventive composition, nanocomposition, and/or pharmaceutical composition.

In yet another embodiment, the invention provides a method for at least one of treating, diagnosing, and preventing a condition in a subject in need thereof. The method includes providing the inventive pharmaceutical composition and administering and/or applying the inventive pharmaceutical composition to the subject, thereby treating, diagnosing, or preventing the condition in the subject. The subject is contemplated as an animal or human but is preferably a human having a human health condition wherein the human health condition is inflammation or is associated with inflammation. Further, the human health condition is often, or is associated with, infection and/or tissue damage. Non-limiting examples of a human health condition are a periprosthetic joint infection (PJI) and a neurodegenerative disease, such as, but not limited to, Alzheimer's disease.

In an embodiment, the invention provides therapeutic and/or pharmaceutical compositions including cerium oxide nanoparticles coated with at least one extracellular protein isolated from Lactobacillus acidophilus (L. acidophilus). Extracellular proteins isolated from Lactobacillus acidophilus are referred to herein as LaEPs. Various, non-limiting examples of LaEPs are listed in Table 1. These therapeutic and/or pharmaceutical compositions are osteoinductive and can directly induce bone formation in cells or tissues. In formulation of these therapeutic and/or pharmaceutical compositions the at least one extracellular protein is added (to the composition) in an amount ranging from about 20 g/mL to about 60 μg/mL. Non-limiting, exemplary concentrations include 20 μg/mL, 40 μg/mL, and 60 μg/mL.

As used herein, the terms “inventive composition” and “inventive nanocomposition” are contemplated as referring to any and all of the various compositions, nanocompositions, pharmaceutical compositions, and therapeutic compositions described herein. These inventive compositions and inventive nanocompositions can be used in any of the methods and/or procedures described herein.

In an embodiment, the invention provides a method for transforming stem cells into osteoblast cells. The method includes providing the inventive nanocomposition and administering the inventive nanocomposition to the stem cells, thereby transforming the stem cells into osteoblast cells. A preferred, non-limiting example of stem cells is human-derived mesenchymal stem cells.

In another embodiment, the invention provides a method for stimulating deposition of bone minerals in cells or tissue. The method includes providing the inventive nanocomposition and administering the inventive nanocomposition to the cells or to the tissue, thereby stimulating deposition of bone minerals in the cells or in the tissue.

In yet another embodiment, the invention provides a method for stimulating new bone formation in cells or tissue. The method includes providing the inventive nanocomposition and administering the inventive nanocomposition to the cells or tissue, thereby stimulating new bone formation in the cells or in the tissue.

In an embodiment, the invention provides a method for inducing osteogenesis in cells or tissue. The method includes providing the inventive nanocomposition and administering the inventive nanocomposition to the cells or to the tissue, thereby inducing osteogenesis in the cells or in the tissue.

In another embodiment, the invention provides a method for decreasing expression of at least one pro-inflammatory gene in cells or tissue. The method includes providing the inventive nanocomposition and administering the inventive nanocomposition to the cells or to the tissue, thereby decreasing expression of the at least one pro-inflammatory gene in the cells or in the tissue. A non-limiting example of a pro-inflammatory gene is RANKL (receptor activator of nuclear factor Kappa-B ligand).

In yet another embodiment, the invention provides a method for increasing expression of at least one anti-inflammatory gene in cells or tissue. The method includes providing the inventive nanocomposition and administering the inventive nanocomposition to the cells or to the tissue, thereby increasing expression of the at least one anti-inflammatory gene in the cells or in the tissue. Non-limiting examples of anti-inflammatory genes are TGF-β (transforming growth factor beta), IL-10 (interleukin-10), CD-163 (Cluster of Differentiation-163), and CD-206 (Cluster of Differentiation-206).

In an embodiment, the invention provides a method for healing or repairing injured tissues by stimulating new bone formation in injured tissues. The method includes providing the inventive nanocomposition and administering the inventive nanocomposition to the injured tissue, thereby stimulating new bone formation in the injured tissue to heal or repair the injured tissue.

In another embodiment, the invention provides a method for protecting cells and tissue from radiation exposure. The method includes providing the inventive nanocomposition and administering the inventive nanocomposition to the cells and tissue, thereby protecting the cells and tissue from radiation exposure.

In yet another embodiment, the invention provides a method for protecting cells and tissue from effects of aging. The method includes providing the inventive nanocomposition and administering the inventive nanocomposition to the cells and tissue, thereby protecting the cells and tissue from the effects of aging.

The above-listed aspects are exemplary embodiments only and are not meant to limit the invention. Other objectives and advantages of this invention will become apparent from the following description taken in conjunction with the accompanying drawings and data, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present invention may be obtained by reference to the accompanying drawings/data when considered in conjunction with the subsequent detailed description. The embodiments illustrated in the drawings are intended only to exemplify the invention and should not be construed as limiting the invention to the illustrated embodiments.

FIGS. 1-11: Experimental Example 1—extracellular proteins isolated from L. acidophilus (LaEPs)

FIGS. 1A-K show data pertaining to whole-cell planktonic monocultures and Staphylococcus aureus growth in co-culture with Lactobacillus acidophilus. Representative TEM images showing monocultured [FIG. 1A]S. aureus, and [FIG. 1B]L. acidophilus species after 24 h. [FIGS. 1C-E] Co-cultured analyses reveal cross-species adhesion and aggregation of S. aureus onto L. acidophilus. [FIG. 1F]% aggregation, [FIG. 1G] zeta potential, [FIG. 1H] particle/aggregate size, and [FIG. 1I] the PDI (poly dispersity index) were measured and compared using a Zetasizer following 0, 2, 4, 6, 18 and 24 h of inoculation. [FIG. 1J] CFU analysis demonstrates the inhibitory activity of L. acidophilus when co-cultured with S. aureus. S. aureus growth is significantly reduced, while L. acidophilus growth is not significantly affected. Statistical analyses: significant differences between experimental groups compared to PBS (control) are represented by an asterisk (*), compared to LA are represented by a [symbol] , and compared to SA are represented by a [symbol] . In all groups, [symbol] <0.05, [symbol] <0.01, [symbol] <0.001, [symbol] <0.0001. Qualitative analysis of whole-cell planktonic S. aureus supplemented with extracellular proteins (200 μg/mL) isolated from the cell free supernatant of monocultured L. acidophilus after 24 h. [FIG. 1K] Representative TEM micrographs of control (untreated), monocultured S. aureus showing the circular cocci shape of individual cells. Representative TEM micrographs show an increase in aspect ratio in the LaEP-treated group, where the cells appear to flatten in shape in response to supplementation with the proteins. The yellow annotations highlight changes in the individual shape patterns observed. Experiments were carried out in triplicate and data is presented as mean±standard error.

FIGS. 2A-L show data pertaining to the effect of extracellular proteins isolated from L. acidophilus (LaEPs) on E. coli, S. aureus, and MRSA planktonic growth and biofilm formation. L. acidophilus extracellular proteins (20, 40 and 60 μg/mL⁻¹) were bacteriostatic towards [FIG. 2A]E. coli, [FIG. 2B]S. aureus and [FIG. 2C] MRSA activity, and when compared to the untreated control group. Inoculations of 10⁴ CFU/mL were cultured for 24 h at 37 °C in phosphate buffered saline (PBS). The number of growing cells was enumerated (number of CFUs) after 24 h of treatment. To investigate biofilm formation [FIG. 2D(a)]E. co/i, [FIG. 2E(a)]S. aureus and [FIG. 2F(a)] MRSA (10⁴ CFU/mL) were each co-cultured with LaEPs at concentrations of 20, 40 and 60 μg/mL⁻¹ in LB for 24 h at 37 °C, and under static conditions. The biofilm formed was stained with 0.1% crystal violet and levels quantified @OD 570 nm. [FIG. 2D(b), FIG. 2E(b), FIG. 2F(b)] Representative images of crystal violet staining in each well within a 96-well plate. The LaEPs significantly inhibited biofilm formation in all bacterial species investigated. Fluorescent microscopy images of [FIG. 2G]E. co/i, [FIG. 2H]S. aureus, and [FIG. 2I] MRSA treated with LaEPs (20, 40, or 60 μg/mL⁻¹). The biofilm was stained with Syto-9 (green) and PI (red) and shows z-stack imaging of biofilm taken at intervals of 6 μm. Only green/viable cells were present, and no dead/red cells were observed in any group (Scale=200 μm). Mean biofilm thickness was quantified in the [FIG. 2J]E. co/i, [FIG. 2K]S. aureus, and [FIG. 2L] MRSA groups with and without treatment with LaEPs in LB broth. A significant decrease in biofilm thickness was observed in all groups and at all LaEP concentrations when compared with control samples. All experiments were performed in triplicate, and data is presented as mean±standard error. *p<0.05, **p<0.01, ***p<0.001, and ****p<0.0001.

FIGS. 3A-G show data establishing that LaEPs are non-cytotoxic, and neglect to induce DNA damage to RAW 264.7 cells following 1 day and 3 days of culture. Cells were grown in DMEM medium and supplemented with LaEPs. [FIGS. 3A(a)-(b)] Cell metabolic activity was assessed and the response to LaEPs quantified using a MTT assay on (FIG. 3 A(a)) day 1, and (FIG. 3A(b)) day 3 of culture. [FIG. 3B] Cell viability was assessed using a live/dead assay on day 1, and [FIG. 3C] day 3. Live cells stained green, and dead cells stain red. No dead cells were noted at these timepoints. [FIG. 3D] Cytoskeletal staining with phalloidin (red) and DAPI (blue) show the cytostructure in each of the groups, and the LaEP-induced formation of an M2-like morphology (yellow arrows) on day 1, and [FIG. 3E] day 3. [FIG. 3F(a)]A Comet assay was used to measure single and double stranded DNA damage on day 1 and, [FIG. 3G(a)] day 3. The DNA parameters were analyzed using Image J software. The levels of DNA damage were expressed as, (b) tail DNA (%), (c) tail moment, and (d) tail length (μm) [FIGS. 3F(b)-(d), Day 1; FIGS. 3G(b)-(d), Day 3]. The LaEPs do not induce DNA damage in macrophages. All experiments were performed in triplicate and data is presented as mean±standard error [FIGS. 3A(a)-(b)] and as box plots [FIGS. 3F(b)-(d); FIGS. 3G(b)-(d)]. *p<0.05, **p<0.01, ***p<0.001, and ****p<0.0001.

FIGS. 4A-F show data establishing that LaEPs regulate macrophage phenotype and modulate both anti- and pro-inflammatory gene expression. Osteoclastic differentiation and activation were not observed. [FIGS. 4A(a)-(h)] qRT-PCR showing gene expression of anti-inflammatory (FIG. 4A(a) TGFβ, FIG. 4A(b) IL-10, FIG. 4A(c) CD163, FIG. 4A(d) CD206) and pro-inflammatory (FIG. 4a(e) TNFα, FIG. 4A(f) IL-6, FIG. 4A(g) IL-1β, FIG. 4A(h) iNOS) marker expression, on day 1, and [FIGS. 4B(a)-(h)] day 3. [FIG. 4C(a)-(b)] osteoclastic (FIG. 4C(a) RANKL and FIG. 4C(b) CTSK) marker expression on day 1, and [FIG. 4D(a)-(b)] day 3. [FIG. 4E] When supplemented with LaEPs at all concentrations, macrophage phenotype altered towards a larger and spindle shape characteristic of the M2 pro-healing subtype on day 1, and [FIG. 4F] day 3. Some macrophages appeared ‘activated’ as determined by the presence of phagosomes (red arrows). TRAP⁺ cells are identified on both day 1 and day 3. All experiments were performed in triplicate and data is presented as mean±standard error. *p<0.05, **p<0.01, ***p<0.001, and ****p<0.0001.

FIGS. 5A-F show data establishing the effect of LaEPs on RAW 264.7 macrophages under LPS (lipopolysaccharide)-simulated chronic inflammatory culture conditions on days 1 and 3. [FIG. 5A] Cells were cultured in DMEM medium supplemented with LaEPs (20, 40, and 60 μg/mL⁻¹) and LPS (10 ng/mL⁻¹), and cell metabolic activity was assessed and compared on day 1, and [FIG. 5B] day 3. [FIG. 5C] Cytoskeletal staining using phalloidin (red) and DAPI (blue) shows the cytostructure in each of the groups on day 1, and [FIG. 5D] day 3. [FIG. 5E(a)]A Comet assay was used to detect DNA damage, and the DNA parameters were analyzed using Image J software on day 1, and [FIG. 5F(a)] day 3. The level of DNA damage expressed as, (b) tail DNA (%), (c) tail moment, and (d) tail length (μm) [FIGS. 5E(b)-(d), Day 1; FIGS. 5F(b)-(d), Day 3). All experiments were performed in triplicate and data is presented as mean±standard error in FIGS. 5A-B, and as box plots for FIGS. 5E(b)-(d); FIGS. 5F(b)-(d). *p<0.05, **p<0.01, ***p<0.001, and ****p<0.0001.

FIGS. 6A-G show the pro- and anti-inflammatory gene expression in macrophages under LPS-simulated chronic inflammatory conditions, with and without LaEPs on days 1 and 3 of culture. [FIGS. 6A(a)-(h)] qRT-PCR showing gene expression of anti-inflammatory (FIG. 6A(a) TGFβ, FIG. 6A(b) IL-10, FIG. 6A(c) CD163, FIG. 6A(d) CD206) and pro-inflammatory (FIG. 6A(e) TNFα, FIG. 6A(f) IL-6, FIG. 6A(g) IL-1β, FIG. 6A(h) iNOS) marker expression on day 1, and [FIGS. 6B(a)-(h)] day 3. [FIGS. 6C(a)-(b)] osteoclastic (FIG. 6C(a) RANKL and FIG. 6C(b) CTSK) marker expression on day 1, and [FIGS. 6D(a)-(b)] day 3. A trend was seen where the increased LaEP dose resulted in lower pro-inflammatory, and higher anti-inflammatory expression. [FIG. 6E] TRAP⁺ cells were detected in the LaEP-given groups, suggesting the presence of activated macrophages on day 1, and [FIG. 6F] day 3. [FIG. 6G] Representative image following examination using transmission light microscopy following a LaEP dose of 20 μg/mL. Macrophages become spindle shaped and extend their pseudopodia to communicate with adjacent cells (yellow arrows). All experiments were performed in triplicate and data is presented as mean±standard error. *p<0.05, **p<0.01, ***p<0.001, and ****p<0.0001.

FIGS. 7A-C show data establishing that isolated L. acidophilus extracellular proteins are non-cytotoxic to hBMSCs. [FIG. 7A]A significant increase in cell metabolic activity was demonstrated on day 3 in the 20 g/mL⁻¹ group only (p<0.001). [FIG. 7B] Cell viability was assessed using a live/dead assay, where live cells stain green, and dead cells, red. [FIG. 7C] Cytoskeletal staining using phalloidin (red) and DAPI (blue) shows similarities in cytostructure in each of the groups. Similar to macrophage results, LaEPs were found to be non-cytotoxic to hBMSCs at the concentrations, and 3-day timepoints investigated. Experiments were carried out in triplicate and data is presented as mean±standard error. ***p<0.001

FIGS. 8A-I show data establishing that isolated L. acidophilus extracellular proteins are osteoinductive and promote osteogenic differentiation and mineralization. [FIG. 8A] Alkaline phosphatase (ALP) activity was quantified in hBMSCs cultured in DMEM without osteogenic media and following supplementation with LaEPs at concentrations of 20, 40 and 60 μg/mL⁻¹. [FIG. 8B]A dark bluish-purple precipitation represents ALP-stained cells, suggesting LaEPs are osteoinductive. [FIG. 8C] ALP activity in hBMSCs when cultured in osteogenic media was quantified. [FIG. 8D] ALP-stained cells in osteogenic media, showing the dark bluish-purple ALP⁺ precipitation. [FIG. 8E] Quantification of Alizarin Red staining (ARS) of hBMSCs cultured in DMEM without osteogenic media. [FIG. 8F] No ARS mineral deposits (red) are observed in cells without lineage differentiation via osteogenic media. This shows LaEPs do not intrinsically augment bone mineral deposition. [FIG. 8G] Quantification of ARS stained hBMSCs following culture in osteogenic media. [FIG. 8H] ARS mineral deposits stain red. LaEPs augment bone mineralization in the presence of osteogenic media. (FIGS. 8I(a)-(j)) hBMSC osteogenic-related gene expression following culture in osteogenic media with LaEPs. All experiments were performed in triplicate and data is presented as mean±standard error. *p<0.05, **p<0.01, ***p<0.001, and ****p<0.0001.

FIGS. 9A-C show data establishing that isolated L. acidophilus extracellular proteins reduce hBMSC adipogenic differentiation in a dose-dependent manner. [FIG. 9A(a)] Quantification of hBMSC intracellular lipid droplet accumulation using ORO staining assay and following adipogenic differentiation. FIG. 9A(b) Representative images of ORO staining in each of the groups. [FIG. 9B] Representative fluorescence microscopy images of accumulated lipid droplets following treatment with and without LaEPs. [FIGS. 9C(a)-(k)] hBMSC adipogenesis-related gene expression following culture in adipogenic differentiating media with and without LaEPs. Experiments were carried out in triplicate and data is presented as mean±standard error. *p<0.05, **p<0.01, ***p<0.001, ****p<0.0001.

FIGS. 10A-B show the proteomic profile of LaEPs. [FIG. 10A]1D SDS-PAGE gel analysis with silver staining of LaEPs at concentrations of 20, 40, and 60 μg/mL⁻ identifies higher intensity protein bands ~10-13 kDa in molecular weight, with weaker intensity bands evident at ~26, 48, 75, and 90 kDa. Putative data obtained from nano-LC-MS/MS analysis suggests higher abundance of the proteins (gene name) rplL (13.4 kDa), pyrH (25.5 kDa), prpL (48.1 kDa), urdA (63.5 kDa), and celE (90.2 kDa). These proteins likely contribute to the high intensity seen within the SDS-based protein bands observed. [FIGS. 10B(a)-(c)] Gene ontology (GO) analysis of the data retrieved from nano-LC-MS/MS shows the biological processes (BP), cellular component (CC), and molecular functions (MF) of LaEPs at a concentration of 60 μg/mL⁻¹.

FIG. 11 shows the proteomic profile, via nano-LC-MS/MS, of a 60 μg/mL⁻¹ concentration of LaEPs; a heat map showing the top 50 abundant proteins (#PSM) by gene name, and the BP, CC, and MF functions associated with each protein.

FIGS. 12-17: Experimental Example 2—LaEPs/CNPs: cerium oxide nanoparticles (CNPs) coated with extracellular proteins isolated from gut probiotic Lactobacillus acidophilus (LaEPs)

FIG. 12* shows the effect of LaEPs-CNPs (cerium oxide nanoparticles (CNPs) coated with extracellular proteins isolated from gut probiotic Lactobacillus acidophilus (LaEPs) on macrophage metabolic activity.

FIG. 13* shows the effect** of LaEPs-CNPs on amounts of the pro-inflammatory factor interleukin-1β (IL-1β).

FIG. 14* shows the effect** of LaEPs-CNPs on amounts of the pro-inflammatory factor interleukin-6 (IL-6).

FIG. 15* shows the effect** of LaEPs-CNPs on amounts of the pro-healing factor transforming growth factor β (TGFβ).

FIG. 16* shows the effect** of LaEPs-CNPs on amounts of the pro-healing factor interleukin-10 (IL-10).

FIG. 17* shows the effect** of LaEPs-CNPs on amounts of the macrophage activator cathepsin K (CTSK).

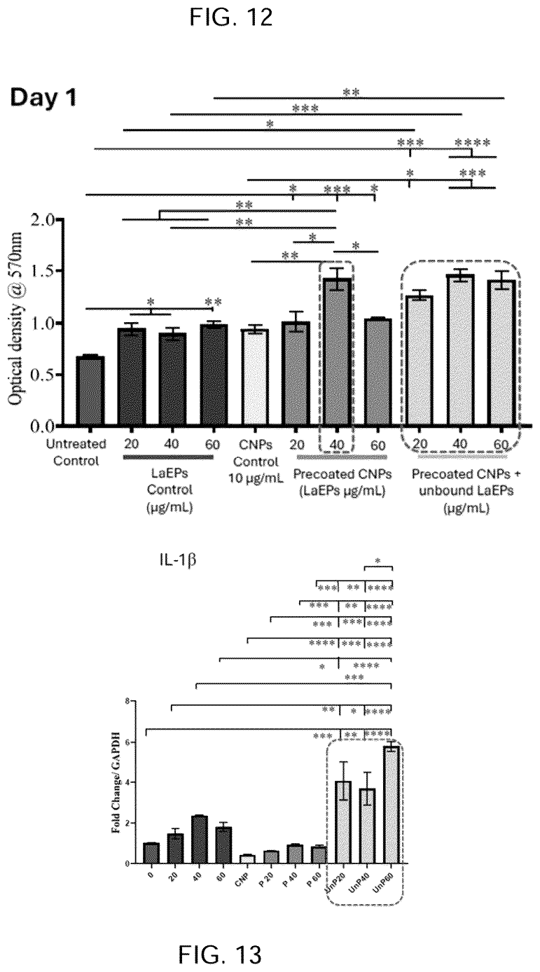

Graphs* of FIGS. 12-17: first five columns left to right represent experimental controls, untreated control, LaEPs controls (20 μg/ml, 40 μg/ml, and 60 μg/ml), CNPs control (10 μg/ml) and next six columns left to right represent tested nanocompositions, coated CNPs (20 μg/ml LaEPs, 40 μg/ml LaEPs, and 60 μg/ml LaEPs) and coated CNPs+unbound LaEPs 20 μg/ml LaEPs, 40 μg/ml LaEPs, and 60 μg/ml LaEPs). The terms “precoated CNPs”, “coated CNPs”, and “LaEPs/CNPs” are used interchangeably herein to refer to nanoparticles having LaEPs coated thereon. The nanoparticles can be partially or completely covered with the LaEPs.

Graphs**of FIGS. 13-17: macrophages were pretreated with lipopolysaccharide (LPS) to simulate a chronic inflammatory condition akin to that seen in the human body.

FIGS. 18-26: Experimental Example 3 (part A-part B)— LaEPs/CNPs: cerium oxide nanoparticles (CNPs) coated with extracellular proteins isolated from gut probiotic Lactobacillus acidophilus (LaEPs) boost new bone formation (part A) and modulate both anti-inflammatory and pro-inflammatory gene expression (part B)

### Part A:

FIGS. 18A-B show data derived from an osteogenesis experiment conducted on day 14 of culture without (in absence of) osteogenic medium. [FIG. 18A] is a graph showing alkaline phosphatase activity on day 14 of culture. *p<0.05, **p<0.01, ***p<0.001, ****p<0.0001. [FIG. 18B] shows micrographs (in both high and low magnification) indicating osteogenesis (blue stain) present in samples measured in FIG. 18A.

FIGS. 19A-B show data derived from a bone mineral deposition experiment conducted on day 28 of culture without (in absence of) osteogenic medium. [FIG. 19A] is a graph showing optical density @415 nm on day 28 of culture. *p<0.05, **p<0.01, ***p<0.001, ****p<0.0001. [FIG. 19B] shows micrographs indicating bone mineral deposition (red stain) in samples measured in FIG. 19A. Red stain=bone mineral deposition.

FIGS. 20A-B show data derived from a bone mineral deposition experiment conducted on day 14 of culture with (in presence of) osteogenic medium. [FIG. 20A] is a graph showing optical density @415 nm on day 14 of culture. *p<0.05, **p<0.01, ***p<0.001, ****p<0.0001. [FIG. 20B] shows micrographs indicating bone mineral deposition (red stain) in samples measured in FIG. 20A. Red stain=bone mineral deposition.

FIGS. 21A-B show data derived from a bone mineral deposition experiment conducted on day 28 of culture with (in presence of) osteogenic medium. [FIG. 21A] is a graph showing optical density @415 nm on day 28 of culture. *p<0.05, **p<0.01, ***p<0.001, ****p<0.0001. [FIG. 21B] shows micrographs indicating bone mineral deposition (red stain) in samples measured in FIG. 21A. Red stain=bone mineral deposition.

### Part B:

FIGS. 22A-B show data derived from a RANKL (receptor activator of nuclear factor Kappa-B ligand) gene expression measurement experiment using quantitative PCR (qPCR). [FIGS. 22A-B] are graphs showing gene expression on day 1 [FIG. 22A] and on day 3 [FIG. 22B] of culture. *p<0.05, **p<0.01, ***p<0.001, ****p<0.0001.

FIGS. 23A-B show data derived from a TGFβ (transforming growth factor beta) gene expression measurement experiment using quantitative PCR (qPCR). [FIGS. 23A-B] are graphs showing gene expression on day 1 [FIG. 23A] and on day 3 [FIG. 23B] of culture. *p<0.05, **p<0.01, ***p<0.001, ****p<0.0001.

FIGS. 24A-B show data derived from an IL-10 (interleukin-10) gene expression measurement experiment using quantitative PCR (qPCR). [FIGS. 24A-B] are graphs showing gene expression on day 1 [FIG. 24A] and on day 3 [FIG. 24B] of culture. *p<0.05, **p<0.01, ***p<0.001, ****p<0.0001.

FIGS. 25A-B show data derived from a CD-163 (cluster of differentiation-163) gene expression measurement experiment using quantitative PCR (qPCR). [FIGS. 25A-B] are graphs showing gene expression on day 1 [FIG. 25A] and on day 3 [FIG. 25B] of culture. *p<0.05, **p<0.01, ***p<0.001, ****p<0.0001.

FIGS. 26A-B show data derived from a CD-206 (cluster of differentiation-206) gene expression measurement experiment using quantitative PCR (qPCR). [FIGS. 26A-B] are graphs showing gene expression on day 1 [FIG. 26A] and on day 3 [FIG. 26B] of culture. *p<0.05, **p<0.01, ***p<0.001, ****p<0.0001.

FIGS. 27A-B show data from another experimental example of biofilm formation prevention of CNPs in the presence of LaEPs with FIG. 27A measured against P. aeruginosa and FIG. 27B measured against S. aureus.

### DETAILED DESCRIPTION OF THE INVENTION

As required, embodiments are disclosed herein; however, it is to be understood that the disclosed embodiments are merely examples and that the nanocompositions, compositions, nanoparticles, therapeutics, pharmaceutical compositions, kits, implants, and/or methods described herein can be embodied in various forms. Therefore, specific structural and functional details disclosed herein are not to be interpreted as limiting, but merely as a representative basis for teaching one skilled in the art to variously employ the instant subject matter in virtually any appropriately detailed structure and function. Further, the terms and phrases used herein are not intended to be limiting, but rather, they are intended to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, or C; A and B; A and C; B and C; and A, B, and C.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Note that not all of the activities described in the general description, or the examples are required, that a portion of a specific activity may not be required, and that one or more further activities can be performed in addition to those described. Still further, the order in which activities are listed is not necessarily the order in which they are performed.

Reference will now be made to specific embodiments illustrated herein and specific language will be used to describe the same. It will nevertheless be understood that no limitation of the scope of the invention is thereby intended. Any alterations and further modification in the described nanocompositions, compositions, nanoparticles, therapeutics, pharmaceutical compositions, kits, implants, and/or methods along with any further application of the principles of the invention as described herein, are contemplated as would normally occur to one skilled in the art to which the invention relates.

The terms “study”, “experiment”, and “experimental example” are used interchangeably herein.

### Summary: Experimental Example 1

Periprosthetic joint infection (PJI) is a challenging complication that can occur following joint replacement surgery. Efficacious strategies to prevent and treat PJI and its recurrence remain elusive, and a novel resolution is urgently needed. Commensal bacteria within the gut microbiota convey beneficial effects by contributing to a defense strategy named “colonization resistance” thereby preventing pathogenic infection and overgrowth along the intestinal surface. This blueprint may be applicable to PJI. Here in this experiment described below, the aim was to investigate Lactobacillus acidophilus spp. and primarily, isolated extracellular-derived L. acidophilus proteins (LaEPs) on PJI relevant S. aureus, MRSA, and E. coli planktonic growth and biofilm formation in vitro. The effect of LaEPs on cultured RAW264.7 macrophages and osteogenic, and adipogenic hBMSC differentiation was also analyzed. Data shows electrostatically induced probiotic-pathogen species co-aggregation and pathogenic growth inhibition together with LaEP-induced biofilm prevention. LaEPs activated macrophages priming them for enhanced microbial phagocytosis via cathepsin K, reduced lipopolysaccharide-induced DNA damage and RANKL expression, and promoted a reparative M2 macrophage morphology when under chronic inflammatory conditions. Proteomic analyses highlight high abundance of lysyl endopeptidase, urocanate reductase, uridylate kinase, large ribosomal subunit protein bL12, and polyketide synthase. Data also shows LaEPs significantly augmented bone deposition while abating adipogenesis thus holding promise as a potential multimodal therapeutic strategy for prevention and treatment of infections such as PJIs.

### Experimental Example 1-Extracellular Proteins Isolated from L. acidophilus as an Osteomicrobiological Therapeutic Agent to Reduce Pathogenic Biofilm Formation, Regulate Chronic Inflammation, and Augment Bone Formation in Vitro¹⁷⁰

### Introduction

Periprosthetic joint infection (PJI) is a challenging complication that can occur following joint replacement surgery and is one of the leading causes of total knee, and total hip arthroplasty (TKA and THA, respectively) failure¹⁻⁴. Periprosthetic infections are reported to occur in 1% to 2% of primary, and 4% of revision arthroplasties⁵⁻⁷, and are associated with high morbidity, patient dissatisfaction, and the need for complex interdisciplinary treatment strategies^{8,9}. Further, the emergence of antimicrobial resistance exacerbates the difficulty in treating PJI^{10,11}. As the demand for primary and revision joint surgeries continues to rise at an accelerated rates,^{5,12,13,169}, an increasing future PJI burden is anticipated, which will have significant healthcare and economic implications. However, efficacious approaches to prevent and treat PJI and its recurrence remain elusive, predominantly due to bacterial adhesion, and subsequent biofilm formation on the implant and surrounding tissue surfaces^{14,15}. Biofilms are comprised of a protective extracellular polymeric substance (EPS) that surrounds bacterial communities, barricading them from adverse or detrimental environmental stressors, including antibiotics, antimicrobial therapies, and host-induced immune countermeasures¹⁶. As such, and once formed, biofilms are notoriously challenging to treat and are currently considered one of the most important factors in implant component-associated infections^{14,17}. To this end, microbial biofilm is associated with ~1.7 million hospital acquired infections per year in the United States, incurring an annual economic burden of ~$11 billion¹⁸.

In the hospital setting, the most common pathogens associated with PJI include 15 Staphylococcus aureus and Staphylococcus epidermidis^{19,20}, with the risk of bacteraemia with S. aureus reported to be up to 34%²¹. Remarkably, Moran et al.²² demonstrated that methicillin-resistant S. aureus (MRSA) can account for up to 74% of PJI, where successful clinical control ranges between 48%-89% in THA^{23,24}, and 60%-74% in TKA^{25,26}. Further, multi-resistant Gram-negative bacteria including Escherichia coli, Klebsiella pneumoniae, Acinetobacter baumannii, and Pseudomonas aeruginosa have also emerged as serious concerns²⁰. The longer the infection persists, the greater the risk of complications, such as implant loosening, joint damage, and systemic spread of the infection²⁷.

However, the pathomechanisms leading to prosthetic loosening are not fully understood. Osteoimmunological pathways and a persistent proinflammatory environment are considered pivotal for delayed bone healing²⁸, disease progression, and long-term implant failure²⁹⁻³¹. To this end, recent studies have suggested that various immune cells, including macrophages, monocytes, dendritic cells, neutrophils, and natural killer cells, may also contribute to the proinflammatory-induced manifestation of ‘immunoporosis’, a term recently coined by Srivastava et al^{32,33}. Specifically, prolonged elevation of the cytokines, interleukin-1-beta (IL-1β), IL-6, and tumor necrosis factor-alpha (TNFα), predominantly produced by activated macrophages, upregulate osteoclastogenesis, osteoclast survival, and osteoclast activity³⁰. This occurs via receptor activator nuclear factor kappa beta (NF)-κB, also known as RANK, along with upregulation of its ligand RANKL, which ultimately increases bone resorption^{34,35}. Notably, macrophages can also engage in bone resorptive behavior, although typically low-grade, with fewer pits that are smaller in diameter when compared to osteoclasts³⁶. Additionally, osteoblasts have been shown to increase proinflammatory cytokine expression (IL-6, IL-8, and monocyte chemoattractant protein-1 (MCP-1)) when challenged with planktonic S. epidermidis or S. aureus³⁷. Indeed, TNFα. and IL-1β also stimulate osteoblastic secretion of MCP-1, and prostaglandin E2, which further attracts osteoclast precursors to the area³⁸, and induces osteoclast formation³⁹, respectively. Furthermore, adipokines participate in the development of chronic inflammation⁴⁰, and increased bone marrow adiposity is a common occurrence and major contributor to many chronic pathological bone loss conditions⁴¹⁻⁴⁵. To this end, changes in the functional capacity of mesenchymal stem cells (MSCs), including impaired proliferation, and a switch in lineage commitment toward adipogenic differentiation at the expense of osteoblastogenesis, have been identified as major contributors to bone loss⁴⁶. Therefore, sustained and elevated levels of these cytokines together with RANKL (relative to its decoy osteoprotegerin (OPG)), likely lead to overarching inflammatory bone loss⁴⁷, thereby negatively impacting osteointegration of an implant following PJI⁴⁸.

Given the current challenges, a novel approach is urgently needed to directly address the issue of biofilm, host inflammation, and bone loss, thereby effectively treating PJI. Emerging evidence highlights the significance of the gut microbiota, which hosts ~100 trillion microbes comprising ~1000 species and 28 different phyla⁴⁹. In turn the gut microbiota directly regulates many aspects of host physiology as well as in treating various inflammatory diseases, including those related to bone health via increasing bone mass, and inhibiting bone resorption^{50,51}. However, the exact mechanisms remain elusive. Among probiotics, lactic acid bacteria, particularly Lactobacillus spp., are widely recognized Gram-positive commensals. These species among others, are reported to convey beneficial effects to the host via inhibition in the growth of pathogenic bacteria, inhibiting biofilm formation⁵², as well as modulating the ecology of biofilms⁵³, thereby contributing to a defense mechanism named, “colonization resistance”⁵⁴. This strategy subsequently aids in preventing pathogenic adhesion, colonization, and overgrowth along the intestinal mucosa⁵⁵. The effects are considered mediated by both direct cell competitive exclusion, as well as via the production of lactic acid, hydrogen peroxide, and/or bacteriocins, antimicrobial peptides, lantibiotics, and microcins. Together, these molecules have been shown to confer bacteriostatic and/or bactericidal mechanisms including membrane permeabilization and interference with essential enzymes, gene expression, and via modulation of the host immune system⁵⁶⁻⁵⁹.

The effect of increasing concentrations of isolated extracellular proteins, derived from the cell free supernatant (CFS) of L. acidophilus (LaEPs), on S. aureus, MRSA, and E. coli planktonic growth and biofilm formation, as well as the macrophage response to LaEPs and their regulation of pro-, and anti-inflammatory gene expression, and phenotype when cultured under simulated chronic inflammatory conditions was investigated. Further, at the same LaEP concentrations, their regulatory effect towards osteogenic versus adipogenic differentiation was also determined. Finally, the proteomic profile was determined using SDS-PAGE gel electrophoresis and nano-liquid chromatography tandem mass spectrometry (nano-LC-MS/MS). The results suggest that LaEPs hold promise as a potential bacteriostatic therapeutic agent that impedes pathogenic biofilm formation, beneficially regulates chronic inflammation, promotes a reparative macrophage phenotype, and augments bone deposition over adipogenesis in vitro.

### Materials and Methods

### Bacterial Strains

Lactobacillus acidophilus (L. acidophilus) was obtained from the American Type Culture Collection (ATCC 4356, Manassas, VA, USA), and cultured in de Man, Rogosa, and Sharpe (MRS) broth (BD Difco Laboratories, NJ, USA). Staphylococcus aureus (S. aureus) ATCC 6538, methicillin resistant S. aureus (MRSA) ATCC 33592, and Gram-negative Escherichia coli (E. coli) ATCC 43888 were also procured from the ATCC and cultured in Luria-Bertani (LB) (Sigma, Germany). All strains were grown at 37 °C for 16 h under 200 rpm of agitation. All tested bacteria were stored at −80 °C in 50% glycerol broth.

### Co-Culture Assay

A co-culture assay was used to determine the antimicrobial effect of L. acidophilus. Monocultures of L. acidophilus and S. aureus were grown in MRS and LB broth, respectively, at 37 °C and for 16 h. Then, both L. acidophilus and S. aureus at an inoculation of 10⁸ CFU/mL⁻¹ were equally (1:1) co-incubated in MRS and LB broth (1:1) for 24 h at 37 °C. A monoculture of L. acidophilus and S. aureus in MRS and LB broth (1:1) were used as control samples. The number of S. aureus and L. acidophilus CFUs was determined via plate counting on LB agar and MRS agar respectively.

### Transmission Electron Analyses

The cultures of S. aureus, L. acidophilus, and coculture of L. acidophilus and S. aureus, were evaluated using transmission electron microscope (TEM) analysis. Samples were preliminarily fixed in a 3.0% solution of glutaraldehyde and washed three times with water. After fixation, cells were dehydrated in increasing concentrations of ethanol (30-100%), and for 20 min at each stage. The samples were then deposited onto a copper grid and images were taken using a JEOL TEM-1011 microscope (JEOL, MA, USA).

Zeta (ζ) potential analysis

L. acidophilus and S. aureus were grown overnight at 37 °C in MRS and LB broth for 0-24 h and harvested following centrifugation at 6000 rpm for 10 mins. The collected cells were washed using PBS (Sigma-Aldrich, USA) and then re-suspended to give a final concentration of 10⁸ CFU/mL⁻¹ or OD600 nm=0.2 (Absᵢₙ). Zeta potential measurements were performed in 0.5 mM potassium phosphate buffer solution (pH 7.4) to minimize the influence of pH. Monocultures of L. acidophilus, S. aureus, and a co-culture of L. acidophilus and S. aureus were assessed, and measurements taken at 0, 2, 4, 6, 18 and 24 h post-treatment (n=3). The zeta potential of the bacteria was analyzed at room temperature using a Zetasizer Nano ZS90 (Malvern instrument, Malvern, UK) with a 633 nm helium-neon laser source. To maintain the buffer salt concentration, water was used as a solvent and each sample was run for 100 cycles.

### % Bacterial Aggregation, Particle Size, and Polydispersity Index

An overnight culture of 10⁸ CFU/mL⁻¹ or OD600 nm=0.3 (Absᵢₙ) was prepared as described above. The suspended solution was incubated for 0-24 h (at 37 °C) and the optical absorption of the upper part (Abs_{fi}) was measured (Zetasizer Nano ZS90 (Malvern instrument, Malvern, UK)). The percentage of bacterial aggregation within the monoculture of L. acidophilus, S. aureus, and a co-culture L. acidophilus and S. aureus was calculated according to the following equation:

$$\% Aggregation = [ \frac{( {Abs}_{in} - {Abs}_{fi} )}{( {Abs}_{in} )} ] \times 100$$

The particle size and polydispersity index (PDI) of the samples was measured in a cuvette at a 90-degree scattering angle (Zetasizer Nano ZS90 (Malvern instrument, Malvern, UK)).

Isolation of Extracellular L. acidophilus Proteins L. acidophilus was cultured in MRS broth for 48 h at 37 °C. The cultures were harvested in a refrigerated centrifuge at 10,000×g for 15 min at 4 °C. The extracellular proteins were precipitated from the culture supernatant by the addition of solid ammonium sulphate at 60% saturation. The resultant mixture was centrifuged at 12,557×g for 60 min at 4 °C. The mixture was then dissolved in a minimum volume of 1×PBS (pH 7.2) and dialyzed against the same buffer. The dialyzed protein sample in 1×PBS was then freeze dried (Lyophilizer, Alpha 1-2 LD plus, Christ, Germany) at a temperature of −53 °C, a pressure of 0.09 mbar, for 6 h and stored at −20 °C.
Testing Protocol for Isolated L. acidophilus Extracellular Proteins

Prior to use, the isolated proteins were filter-sterilized by passing the solution through a sterile Uniflo 0.2 lm pore size PVDF filter (Celltreat, USA). The density of each microbial suspension (S. aureus, MRSA and E. coli) was adjusted to 10⁴ CFU/mL⁻¹ prior to testing at concentrations of 20, 40 and 60 μg/ml⁻¹ in phosphate buffered saline (PBS). An inoculum aliquot of 100 μl of each test pathogen was spread over a pre-solidified LB agar plate, and the number of bacteria present was determined by plate counting.

### Biofilm Formation on a Polystyrene Plate

The ability of S. aureus, MRSA, and E. coli to form biofilms following treatment with isolated extracellular L. acidophilus proteins was analyzed. Briefly, at an inoculum of 10⁴ CFU/mL⁻¹, S. aureus, MRSA and E. coli were cultured with LaEPs at a concentration of 20, 40 or 60 μg/mL⁻¹. Cultures without LeEPs served as control. Bacteria were cultured in a 96-well plate (Costar®, Sigma Aldrich) and incubated at 37 °C for 24 h. The plates were then washed twice with PBS, before adding 0.1% of crystal violet. The plates were incubated for a further 30 mins at room temperature. Each well was washed twice with PBS, and 200 μL of 95% ethanol was added prior to measuring absorbance @570 nm using a Spectramax iD3 multimode microplate reader (Molecular Devices, USA). Samples were also stained with Syto 9 and imaged using fluorescent microscopy (Keyence BZ-x800, Keyence, USA). Z-stack imaging (intervals of 6 μm; Keyence, USA) was used to compile XYZ positioned 3D reconstruction of biofilm in each of the groups. Three random images, per each triplicate well, and 10 points per image were used to quantify biomass thickness (μm).

RAW264.7 Macrophage and hBMSC Culture

Murine-derived RAW 264.7 macrophages ATCC® TIB-71™ were sourced from the ATCC. RAW 264.7 cells were maintained in Dulbecco's modified Eagle's medium (DMEM; Thermo Fisher Scientific, USA) supplemented with 10% heat inactivated fetal bovine serum (FBS; Thermo Fisher Scientific, USA), 1% (v/v) penicillin/streptomycin (P/S; Thermo Fisher Scientific, USA) and 2 mM L-Glutamine in a humidified incubator containing 5% CO₂ at 37 °C. Human bone marrow derived mesenchymal stem cells (hBMSCs, ATCC® PCS-500-012™)) were maintained in DMEM supplemented with 10% FBS, and 1% (v/v) P/S at 37 °C.

### Cell Metabolic Activity

Cell metabolic activity was quantified on days 1 and 3 for macrophages, and on day 3 when investigating hBMSCs. Cells were cultured with and without LaEPs at a concentration of 20, 40 or 60 μg/mL⁻ using a MTT [3-(4, 5-dimethylthiazol-2-yl)-2, 5-diphenyl tetrazolium bromide] assay (M2128, Millipore Sigma, USA). RAW 264.7 macrophages were seeded in 96-well plates at a density of 2.5×10³/well and cultured overnight at 37 °C. hBMSCs were seeded in 24-well plates at a density of 1×10⁵/well. On the respective days of culture, 20 μL of a 5 mg/mL MTT solution was added into each well for 4 h. The supernatant was then carefully removed and 100 μL of dimethyl sulfoxide was used to dissolve the formazan crystals. The absorbance was read on a Spectramax iD3 multimode microplate reader (Molecular Devices, CA, USA).

### Analyses of Cell Viability and Morphology

Biocidal activity was analyzed using a Live/Dead® assay (Molecular Probes, Thermofisher, USA). Cells were stained with fluorescein diacetate (FDA)/propidium iodide (PI) and imaged using fluorescent microscopy (Keyence BZ-x800, Keyence, FL, USA). To assess changes in cell cytoskeletal morphology, and following 3 days of culture, cells (RAW264.7 macrophages or hBMSCs) were gently rinsed twice with PBS at a pH 7.4, and stained with Phalloidin/DAPI (Invitrogen, CA, USA). Cell morphology was visualized using fluorescent microscopy.

### DNA Damage

DNA damage was evaluated using single cell gel electrophoresis and the alkaline Comet Assay® kit (R&D systems, MN, USA). Following 3 days of culture, the RAW 264.7 macrophages were pelleted via centrifugation at 1500 g for 5 min at 4 °C. Cells were combined with LMagarose at a ratio of 1:10 (v/v) and spread over a glass slide before being transferred to 4 °C, for 15 mins in the dark. Slides were transferred to lysis buffer containing DMSO for 30-60 min at 4 °C. This was then followed by immersion in alkaline unwinding solution for 2 h at 4 °C, before analysis in pre-chilled alkaline electrophoresis solution. Slides were run at 21 v for 30 mins before being gently rinsed with water twice and immersed in 70% ethanol for 5 min and air dried at 37 °C for 10-15 min. 100 μL of diluted SYBR gold dye was added and samples stained for 30 mins. Samples were dried at 37 °C before being imaged using fluorescence microscopy (Keyence BZ-x800, Keyence, USA). DNA damage was quantified by measuring tail DNA (%), tail moment (arbitrary units), and tail length (μm). All of the comet parameters were measured and analyzed using OpenComet (v1.3.1) in ImageJ.

Quantitative Real-Time Reverse-Transcription Polymerase Chain Reaction (qRT-PCR)

The mRNA expression levels of target genes related to pro-inflammatory, anti-inflammation, and osteoclastic markers were investigated in macrophages, while osteogenic-related, and adipogenic-related gene expression were investigated in hMSCs using qRT-PCR. Total RNA was extracted using the PureLink™ RNA Mini Kit (Thermofisher Scientific, FL, USA). The extracted RNA was reverse-transcribed into cDNA using a SuperScript™ IV VILO™ Master Mix kit (Thermofisher Scientific, FL, USA). qRT-PCR analysis was performed using the Fast SYBR® Green Master Mix (Applied Biosystems, MA, USA). The sequences of qRT-PCR primers used in this experiment are presented in Table 2, SEQ ID NOS:1-62, numbering from top to bottom of table. The fold change of relative mRNA expression was calculated using the comparative Ct (2^{-ΔΔCT}) method.

TABLE 2
Oligonucleotide sequences employed in qPCR.

| | | |
|---|---|---|
| Gene | Primer | Sequence 5′ to 3′ |

Mouse Anti and Pro-inflammatory markers:

| | | |
|---|---|---|
| GAPDH | F | CTCCCACTCTTCCACCTTCG |
| | R | GCCTCTCTTGCTCAGTGTCC |
| TGF-β | F | CAACAATTCCTGGCGTTACCTTGG |
| | R | GAAAGCCCTGTATTCCGTCTCCTT |
| IL-10 | F | TGAATTCCCTGGGTGAGAAG |
| | R | TCACTCTTCACCTGCTCCACT |
| CD163 | F | GGGTCATTCAGAGGCACACTG |
| | R | CTGGCTGTCCTGTCAAGGCT |
| CD206 | F | GTTAGTGTACCGCACCCTCC |
| | R | AAACACAGACTGACCCTTCCC |
| TNFα | F | TGCCTATGTCTCAGCCTCTTC |
| | R | GAGGCCATTTGGGAACTTCT |
| IL-6 | F | GTTCTCTGGGAAATCGTGGA |
| | R | GGAAATTGGGGTAGGAAGGA |
| IL-β | F | AAATACCTGTGGCCTTGGGC |
| | R | CTTGGGATCCACACTCTCCAG |
| iNOS | F | TGTGTTCCACCAGGAGATGT |
| | R | AGGTGAGCTGAACGAGGAA |

Human Osteogenic markers:

| | | |
|---|---|---|
| GAPDH | F | GCATCCTGGGCTACACTGAG |
| | R | GTCAAAGGTGGAGGAGTGGG |
| COL1 | F | ACAAATGAATGGGGAAAGAC |
| | R | TTATTTGAAACAGACTGGGC |
| BGLAP | F | TTCTTTCCTCTTCCCCTTG |
| | R | CCTCTTCTGGAGTTTATTTGG |
| Runx2 | F | GCAGTATTTACAACAGAGGG |
| | R | TCCCAAAAGAAGTTTTGCTG |
| SPP1 | F | GACCAAGGAAAACTCACTAC |
| | R | CTGTTTAACTGGTATGGCAC |
| ALPL | F | ATGTGGAGTATGAGAGTGAC |
| | R | CTTGTATCTCGGTTTGAAGC |
| BMP7 | F | TGGTCCACTTCATCAACC |
| | R | TTCTGTATTTCTTCAGGAGAC |
| BSP | F | GGAGACTTCAAATGAAGGAG |
| | R | CAGAAAGTGTGGTATTCTCAG |
| OCN | F | CACCGAGACACCATGAGAGC |
| | R | CTGCTTGGACACAAAGGCTGC |
| OPN | F | TCTGGGAGGGCTTGGTTGT |
| | R | AACTGGTATGGCACAGGTGA |

Adipogenic markers:

| | | |
|---|---|---|
| GAPDH | F | GCATCCTGGGCTACACTGAG |
| | R | GTCAAAGGTGGAGGAGTGGG |
| C/EBPα | F | AGCCTTGTTTGTACTGTATG |
| | R | AAAATGGTGGTTTAGCAGAG |
| PPARγ | F | AAAGAAAGCCAACACTAAACC |
| | R | TGGTCATTTCGTTAAAGGC |
| LEP | F | TCAATGACATTTCACACACG |
| | R | TCCATCTTGGATAAGGTCAG |
| FASN | F | CAATACAGATGGCTTCAAGG |
| | R | GATGTATTCAAATGACTCAGGG |
| FABP4 | F | CAAGAGCACCATAACCTTAG |
| | R | CTCGTTTTCTCTTTATGGTGG |
| SREBF1 | F | AATCTGGGTTTTGTGTCTTC |
| | R | AAAAGTTGTGTACCTTGTGG |
| LRP4 | F | AAATCCAAGTTCACTGATCC |
| | R | CCTCTTTCTTATAGCACAGC |
| TNFRSF11B | F | ATAGATGTTACCCTGTGTGAG |
| | R | AAGAGACTAAGCCAGTTAGG |
| ADIPOQ | F | GGTCTTATTGGTCCTAAGGG |
| | R | GTAGAAGATCTTGGTAAAGCG |
| LPL | F | ACACAGAGGTAGATATTGGAG |
| | R | CTTTTTCTGAGTCTCTCCTG |
| PLINI | F | CCCTGAAAAGATTGCTTCTG |
| | R | AGGACCTTGTCTGAAGTG |

### Chronic Inflammatory Simulation and TRAP Staining

Macrophages were stimulated with 10 ng/mL⁻¹ of lipopolysaccharide (LPS) in the presence of the extracellular protein of increasing concentrations of LaEP (20, 40 and 60 μg/mL-1) over a 3-day culture period. Mature osteoclasts were validated using Tartrate-resistant acid phosphatase (TRAP) staining according to a previously published protocol⁴¹.

### Osteogenic Differentiation and Mineralization

hBMSCs were seeded in 24-well plates at a density of 1×10⁵/well. The cells were then treated with either 20, 40 or 60 g/mL⁻¹ of LaEPs with or without osteogenic medium, which comprised of the osteogenic components; 2 mM β-glycerophosphate, 100 M L-ascorbic acid 2-phosphate, 10 nM dexamethasone (Sigma-Aldrich, MA, USA)). Untreated hBMSCs served as control samples. Cells were maintained by replenishing media every 3 days. To quantify alkaline phosphatase (ALP) activity, hBMSCs were treated with LaEPs and culture expanded for 14 days. ALP activity was measured on day 14 using an ALP assay kit (Colorimetric, ab83369, Abcam, USA), and performed per the manufacturer's protocol. The hBMSCs were collected and homogenized and the cell lysate incubated with p-nitrophenyl phosphate solution at 25 °C for 60 mins. To end the reaction, 20 μL of stop solution was added and the solution measured using a Spectramax iD5 (Molecular Devices, CA, USA) at 405 nm. To visualize ALP⁺ cells, ALP staining was performed according to the manufacturer's instructions (K2035-50, BioVision Diagnostics, IL, USA). After removing the culture medium, cells were washed in 300 μL washing buffer, and stained with ALP staining solution for 30 min at 37 °C. After rinsing twice with washing buffer, images were captured using a bright field microscope (BZ-X800E, Keyence, FL, USA).

In vitro mineralization in the presence and absence of LaEPs (20, 40 or 60 μg/mL⁻¹), and with or without supplementation with osteogenic differentiation media was determined using Alizarin Red S (ARS) staining. hBMSCs were cultured to 28 days, washed and fixed using 4% PFA for 20 mins at room temperature. After fixation, cells were washed with deionized H₂O and incubated with 2% pH 4.1 ARS solution (A5533-25G, Millipore Sigma, MA, USA) for 20 mins at room temperature. Images of mineral deposits formed in each of the groups were taken using inverted bright field microscope (BZ-X800E, Keyence, FL, USA). Then cells were treated with 500 μL acidic solution (sodium dodecyl sulfate 5% in 0.5 N HCl) for 30 mins to release the cell-bound stain. The absorbance value of the released ARS stain was measured using a Spectramax iD5 (Molecular Devices, CA, USA) plate reader at 415 nm.

### Adipocyte Differentiation

hBMSCs were seeded in 6-well plates at a density of 6×10³/well. Once confluent, the medium was replaced with the differentiation cocktail medium MesenCult™ Adipogenic Differentiation Kit (Human) (#05412, Stem cell technologies, MA, USA). Cells were treated with LaEPs at a concentration of either 0, 20, 40 or 60 μg/mL⁻¹ over a 21-day culture period. Intracellular lipid accumulation in differentiated hBMSCs was measured using Oil Red 0 staining and following the method described by Ramiŕez-Zacariá et al¹⁶⁷. Briefly, differentiated hBMSCs were washed twice and fixed in a 3.7% formaldehyde solution for 15 mins. Subsequently, the cells were washed three times with PBS and then stained with filtered Oil Red O solution for 30 mins at room temperature. Oil Red 0 solution was removed from the wells, and each well was then washed three times with PBS. Oil Red O quantification was performed by extracting the dye with 100% isopropanol, and the absorbance measured at 540 nm using a microplate reader. For fluorescent imaging, fixed cells were stained with LipidSpot™ Lipid Droplet Stain kit (Biotium, CA, USA).

### Sodium Dodecyl Sulfate Poly Acryl Amide Gel Electrophoresis (SDS-PAGE)

LaEPs at a concentration of 20, 40, and 60 μg/mL⁻¹ were electrophoresed on a 10% Tris-Glycine-SDS-polyacrylamide gel. The 10 to 250 kDa protein marker (Precision Plus Protein™ Kaleidoscope™, Biorad, USA) was used to identify the molecular mass of standard reference proteins. The prepared gel was 20.0 cm long and 1.0 mm thick and run at 50 v for an initial 30 mins in 4% stacking gel, followed by 120 v in a 10% separating gel. After electrophoresis, the SDS gel was fixed for 20 mins in a solution of 50% methanol and 5% acetic acid and rinsed with distilled water. The gel was then stained with Ag as described by Green et al¹⁶⁸.

### Nano-Liquid Chromatography Mass Spectrometry of LaEPs

Nano-liquid chromatography mass spectrometry (Nano-LC/MS/MS) analysis of LaEPs was carried out by the Mass Spectrometry Research and Education Center, University of Florida.

### In Solution Digestion

Proteins were extracted and digested using the EasyPep™ MS Sample Prep Kit (Thermo Fisher Scientific, FL, USA). Total protein was determined on a Qubit and the appropriate volume of each sample was taken to equal 20 μg total protein for digestion. The samples were digested with sequencing grade trypsin/lys C rapid digestion kit from Promega (Madison WI) using the manufacturer's recommended protocol. Three times the sample volume of rapid digestion buffer (provided with the kit) was added to the samples. The sample was incubated at 56 °C with 1 μL of dithiothreitol (DTT) solution (0.1 M in 100 mM ammonium bicarbonate) for 30 minutes prior to the addition of 0.54 μL of 55 mM Iodoacetamide in 100 mM ammonium bicarbonate. Iodoacetamide was incubated at room temperature in dark for 30 min. The trypsin/lys C was prepared fresh as 1 μg/μL in the rapid digestion buffer. 1 μL of enzyme was added and the samples were incubated at 70 °C for 1 hour. The digestion was stopped by addition of 0.5% TFA. The MS analysis was immediately performed to ensure high quality tryptic peptides with minimal non-specific cleavage.

### Q Exactive HF Orbitrap

Nano-liquid chromatography tandem mass spectrometry was performed on a Thermo Scientific Q Exactive HF Orbitrap mass spectrometer equipped with an EASY Spray nanospray source (Thermo Scientific, FL, USA) operated in positive ion mode. The LC system was an UltiMate™ 3000 RSLCnano system from Thermo Scientific. The mobile phase A was water containing 0.1% formic acid and the mobile phase B was acetonitrile with 0.1% formic acid. The mobile phase A for the loading pump was water containing 0.1% trifluoracetic acid. 5 mL of sample is injected on to a PharmaFluidics mPAC™ C18 trapping column (C18, 5 m pillar diameter, 10 mm length, 2.5 μm inter-pillar distance) at 10 μL/mL flow rate. This was held for 3 minutes and washed with 1% B to desalt and concentrate the peptides. The injector port was switched to inject and the peptides were eluted off the trap onto the column. PharmaFluidics 110 cm mPAC™ was used for chromatographic separations (C18, 5 μm pillar diameter, 50 cm length, 2.5 μm inter-pillar distance). The column temperature was maintained at 40 °C. A flowrate of 750 nL/min was used for the first 15 minutes and then the flow was reduced to 300 nL/min. As shown below in Table 3, peptides were eluted directly off the column into the Q Exactive system using a gradient of 1% B to 20% B over 100 minutes and then to 45% B in 20 minutes for a total run time of 150 minutes.

TABLE 3
Peptides were eluted using a gradient of % B (acetonitrile
with 0.1% formic acid) at reduced flowrates over
a total run time of 150 minutes.

| Time (min) | % B | Flow Rate (nL/min) |
|---|---|---|
| 0 | 1 | 750 |
| 3 | 1 | 750 |
| 15 | 5 | 750 |
| 15.1 | 5 | 300 |
| 100 | 20 | 300 |
| 123 | 45 | 300 |
| 130 | 95 | 300 |
| 135 | 95 | 300 |
| 135.1 | 1 | 300 |
| 150 | 1 | 300 |

The MS/MS was acquired according to standard conditions established in the lab. The EASY Spray source operated with a spray voltage of 1.5 KV and a capillary temperature of 200 °C. The scan sequence of the mass spectrometer was based on the original TopTen™ method; the analysis was programmed for a full scan recorded between 375-1575 Da at 60,000 resolution and a MS/MS scan at resolution 15,000 to generate product ion spectra to determine amino acid sequence in consecutive instrument scans of the fifteen most abundant peaks in the spectrum. The AGC Target ion number was set at 3e6 ions for full scan and 2e5 ions for MS² mode. Maximum ion injection time was set at 50 ms for full scan and 55 ms for MS² mode. Micro scan number was set at 1 for both full scan and MS² scan. The HCD fragmentation energy (N)CE/stepped NCE was set to 28 and an isolation window of 4 m/z. Singly charged ions were excluded from MS². Dynamic exclusion was enabled with a repeat count of 1 within 15 seconds and to exclude isotopes. A Siloxane background peak at 445.12003 was used as the internal lock mass.

All MS/MS spectra were analyzed using Sequest (Thermo Fisher Scientific, San Jose, CA, USA; version IseNode in Proteome Discoverer 3.0.1.27). Sequest was set up to search Lactobacillus acidophilus (NcbiAV TaxID=2) (v2023-01-24) assuming the digestion enzyme trypsin. Sequest was searched with a fragment ion mass tolerance of 0.020 Da and a parent ion tolerance of 10.0 ppm. Carbamidomethyl of cysteine was specified in Sequest as a fixed modification. Met-loss of methionine, met-loss+Acetyl of methionine, oxidation of methionine and acetyl of the n-terminus were specified in Sequest as variable modifications.

Precursor ion intensity label free quantitation was carried out using Proteome Discoverer (Thermo Fisher Scientific vs 2.4.0.305). Normalization was derived by using all peptides. Protein abundances were calculated by summed abundances, meaning the protein abundances are calculated by summing sample abundances of the connected peptide groups.

A heat map was generated to represent gene expression patterns. Genes were classified based on cellular function and pathways using the functional annotation tool in DAVID (Database for Annotation, Visualization and Integrated Discovery). DAVID databases were used to identify the pathways and gene functions. The significance of the pathway classification was calculated based on the EASE score. The biological process (BP), cellular components (CC), and the molecular function (MF) were sorted based on the number of genes, false discovery rate (FDR), and log P value.

### Statistical Analysis

Statistical analysis was performed using a two-way ANOVA with a post-hoc Tukey's test for comparison between groups (GraphPAD Prism, v8, USA). p values <0.05 were considered significant. Mean values±SE are presented.

### Results

Suppression of Whole-Cell Planktonic Staphylococcus aureus Growth in Co-Culture with Lactobacillus acidophilus

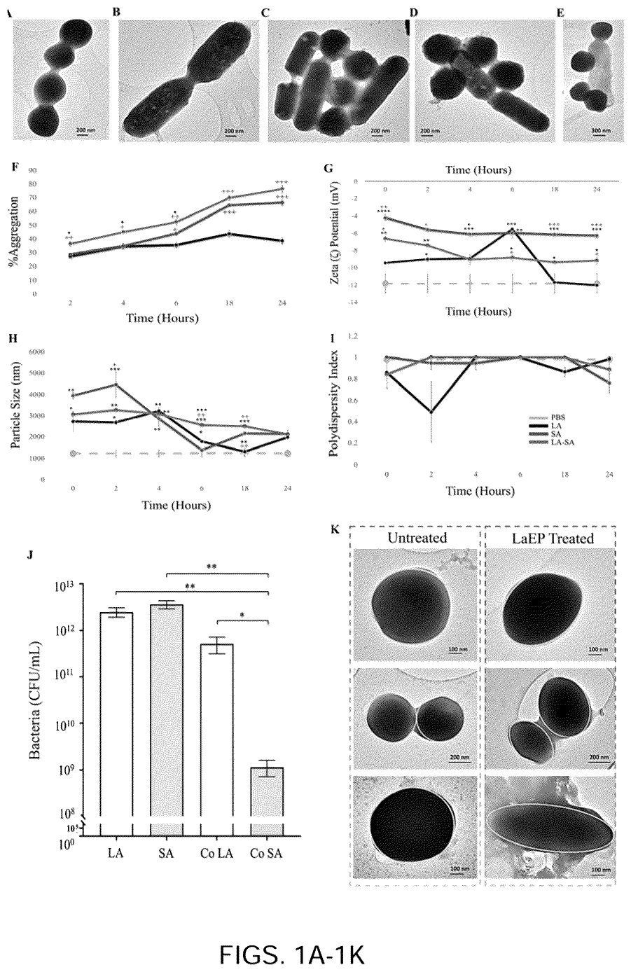

Auto-aggregation is often associated with microcolony and biofilm formation, and thus, pathogenesis in pathogenic bacteria⁶⁰. While in contrast, auto-aggregation and co-aggregation of Lactobacillus strains may infer greater probiotic potential by creating a hostile micro-environment around a pathogen^{61,62}. Specifically, adherence and aggregation largely by hydrophobic Lactobacilli^{63,64}, can inhibit adherence of pathogenic bacteria to the intestinal mucosa by forming a barrier via direct co-aggregation, thereby facilitating their elimination and avoiding colonization⁶⁵⁻⁶⁷. Here, planktonic auto-aggregation of both S. aureus (SA) (FIG. 1A) and L. acidophilus (LA) (FIG. 1B), as well as co-aggregation within a co-culture of L. acidophilus and S. aureus (LA-SA) in MRS and LB broth (1:1) and after a 24 h time-period, were assessed using transmission electron microscopy (TEM) (FIGS. 1C-E). The results show that during co-culture, S. aureus and L. acidophilus species adhere and co-aggregate, often with multiple S. aureus cocci cohered to a single L. acidophilus cell. The percentage of bacterial aggregation in each culture was quantified, and results showed time-dependent aggregation in both the mono- and co-cultures investigated. Specifically, significantly increased auto-aggregation was measured in the pathogenic S. aureus group when compared to commensal L. acidophilus at 6 h (p<0.05), 18 h (p<0.001), and 24 h (p<0.001) timepoints (FIG. 1F). Notably, a significant increase in % aggregation was revealed in the LA-SA co-cultures at all timepoints when compared with the L. acidophilus group (2 h, p<0.01), 4 h, p<0.05, 6 h, p<0.01, 18 h, p<0.001, 24 h, p<0.001), and following 2, 4 and 6 h (p<0.05 in all cases) in the S. aureus monocultured group. Gram-positive S. aureus and L. acidophilus, as well as Gram-negative bacterial species, possess a negatively charged cell wall, and differences in the electrochemical properties of the bacterial surface charge can be described by the zeta (ζ) potential⁶⁸. The results reveal L. acidophilus displayed the most anionic, and significant mean surface ζ potential (−9.47±0.13 mV) when compared to S. aureus (−4.21±0.32 mV, p<0.01). Further, and at the ‘0’ timepoint, a combined LA-SA inoculation resulted in a comparatively centralized ζ potential of −6.66±0.29 (FIG. 1G). Thus, the interaction observed can, in part, be explained via electrostatic interactions, together with likely favorable S. aureus binding to L. acidophilus due to the increased surface area available. Using atomic force spectroscopy, Younes et al⁶². showed that the adhesion forces between Lactobacillus spp. and S. aureus were stronger than between staphylococcal pairs and resulted in significantly larger co-aggregates. The findings reveal that although % aggregation was significantly increased in the LA-SA co-cultured group, the size of the aggregations formed at the earlier timepoint of 2 h were smaller (3264.00±45.03 nm) than the auto-aggregates displayed in S. aureus monocultures (4459.00±584.80 nm), and a similar aggregate size compared with the L. acidophilus (2672.00±114.50 nm) group (FIG. 1H). Although a trend was seen in all groups where aggregate size decreased over time, at the 6 and 18 h timepoints, significantly larger aggregates were measured in the LA-SA co-culture group (2544.00±143.9, and 2486.00±100.9 nm, respectively) when compared with both L. acidophilus (6 h, 1772.00±50.02 p<0.001; and 18 h, 1293.00±162.60 nm p<0.01)) and S. aureus aggregates (6 h only, 1359.00±67.72p<0.001). The dispersity (polydispersity Index PDI) is used to describe the degree of ‘non-uniformity’ of a distribution in size, shape, and mass distribution. A perfectly uniform sample would present with a PDI of 0.0. The data reveal no significant differences when each of the monocultures and co-cultures were compared (FIG. 1I).

Next, whether planktonic whole-cell growth of both species was affected during co-culture was analyzed. Remarkably, the results show L. acidophilus (CoLa) significantly inhibited S. aureus (CoSa) growth by 3-log (p<0.0031) in comparison with monoculture S. aureus at the 24 h time point (FIG. 1J). Notably, no significant differences in growth were determined when each species was cultured individually. Additionally, L. acidophilus growth was not significantly affected by S. aureus when in co-culture and over the 24 h period.

Isolated Extracellular L. acidophilus Proteins Inhibit E. coli, S. Aureus, and MRSA Planktonic Growth and Biofilm Formation

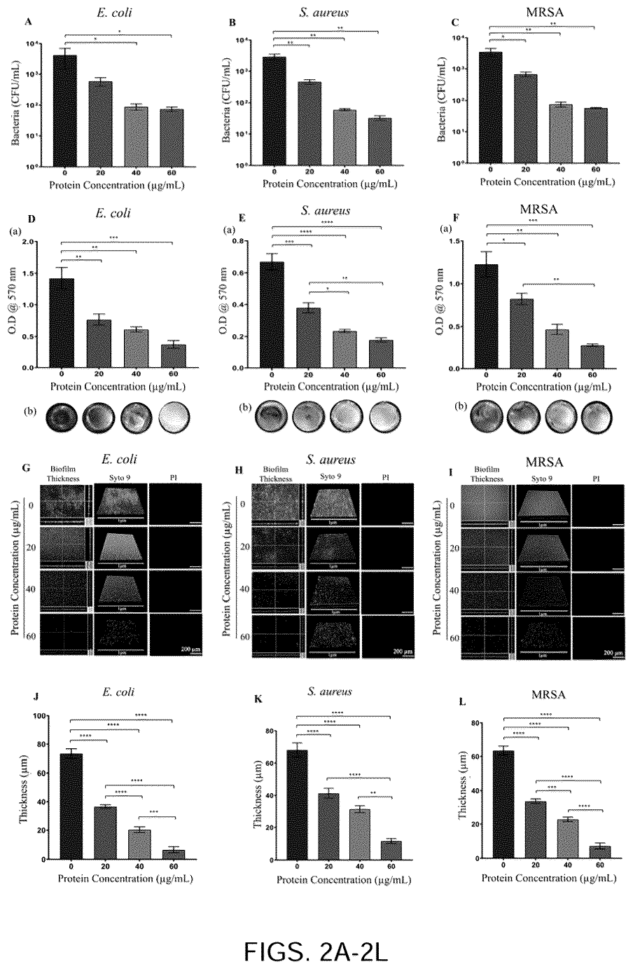

Although uncommon, Lactobacillus spp. bacteremia have been associated with PJI⁶⁹⁻⁷⁰. Nevertheless, the release of bacteriocins, microcins, and other antimicrobial peptides and substances are known to confer bacteriostatic and/or bactericidal mechanisms to pathogenic bacteria as well as modulate the host immune systems^{56-59,71-73}. Therefore, a major focus of this study was the investigation of extracellular protein release from L. acidophilus on challenging pathogenic bacterial species associated with PJI; namely, S. aureus, E. coli, and MRSA. Remarkably, TEM analyses reveal that the supplementation of whole-cell S. aureus with extracellular proteins isolated from the cell free supernatant of monocultured L. acidophilus (LaEPs) resulted in alterations in cell morphology (FIG. 1K). Next, the effect of E. coli, S. aureus, and MRSA growth at increasing LaEP concentrations (20, 40, 60 μg/mL⁻¹, FIGS. 2A-C) was investigated. The bacteriostatic and bactericidal properties of LaEPs were assessed and the results show that as the dose increased in each of the three species investigated, pathogenic bacterial growth decreased in a dose-dependent manner. A significant and ~1.67- and 1.75-fold log 10 reduction in Gram-negative E. coli growth was measured following supplementation with 40, and 60 μg/mL⁻¹ of proteins (p<0.01 in both groups). Further, the LaEPs resulted in a significant and ~1-fold log 10 decrease following administration at 20 μg/mL⁻¹, a ~1.5-decrease at 40 μg/mL⁻¹, and a ~2-fold log 10 decrease at 60 μg/mL⁻¹ (p<0.01 in all groups) when administered to S. aureus. Notably, in the MRSA group, a significant reduction in growth was measured at all three concentrations, with a ~1.5-fold log 10 reduction measured at the 40, and 60 μg/mL⁻¹ dose (p<0.01 in all cases). These results suggest moderate LaEP bacteriostatic properties at the time points investigated.

E. coli, S. aureus, and MRSA are each strong biofilm producers, and here whether LaEPs inhibit biofilm growth was examined. E. coli, S. aureus and MRSA (10⁴ CFU/mL⁻¹) were each co-cultured with LaEPs at concentrations of 20, 40 and 60 μg/mL⁻¹. The results reveal the proteins significantly inhibited biofilm formation in all strains, and at all LaEP concentrations in a dose-dependent manner (FIGS. 2D-F). Fluorescent microscopy and Syto-9 staining confirmed these results. A substantial decrease in biofilm biomass was measured in all three strains with a proportional reduction in live cells observed (FIGS. 2G-I). Biofilm thickness was quantified, and the mean in the E. co/i control group (73.40±3.40 m) was significantly thicker than biofilm formation following co-culture with LaEPs at all concentrations (36.61±1.34 m, 20.36±2.05 m, and 6.67±2.03 m, at 20, 40 and 60 μg/mL⁻¹ respectively. p<0.0001 in all groups (FIG. 2J). Similarly, a significant reduction in biofilm thickness was measured when S. aureus and MRSA were each co-cultured with LaEPs. In the S. aureus group, and when compared with the untreated group, a significant decrease in thickness was measured following culture with 20 μg/mL⁻¹ (41.33±3.15 m), 40 μg/mL⁻¹ (31.40±2.17 m), and at 60 μg/mL⁻¹ of LaEP (11.50±1.50 μm (p<0.0001 in all groups)) (FIG. 2K). The same pattern was observed in the MRSA samples when treated with L. acidophilus isolated extracellular proteins. The mean thickness significantly reduced in the 20 μg/mL⁻¹ (33.65±1.39 μm (p<0.0001), 40 μg/mL⁻¹ (22.80±1.40 m (p<0.0001) and at 60 μg/mL⁻¹ (11.50±1.65 μm (p<0.0001) group following co-culture, and when compared with the control group (FIG. 2L). Thus, LaEPs inhibited bacterial growth and biofilm formation in all three of the bacterial species investigated. Notably, the results also showed no dead cells following treatment, suggesting that at this dose and timepoint, LaEPs are bacteriostatic.

Isolated L. acidophilus Extracellular Proteins Augment Macrophage Metabolic Activity, are Non-Cytotoxic, Neglect to Induce DNA Damage, and Regulate Both Pro-Inflammatory, and Anti-Inflammatory Gene Expression

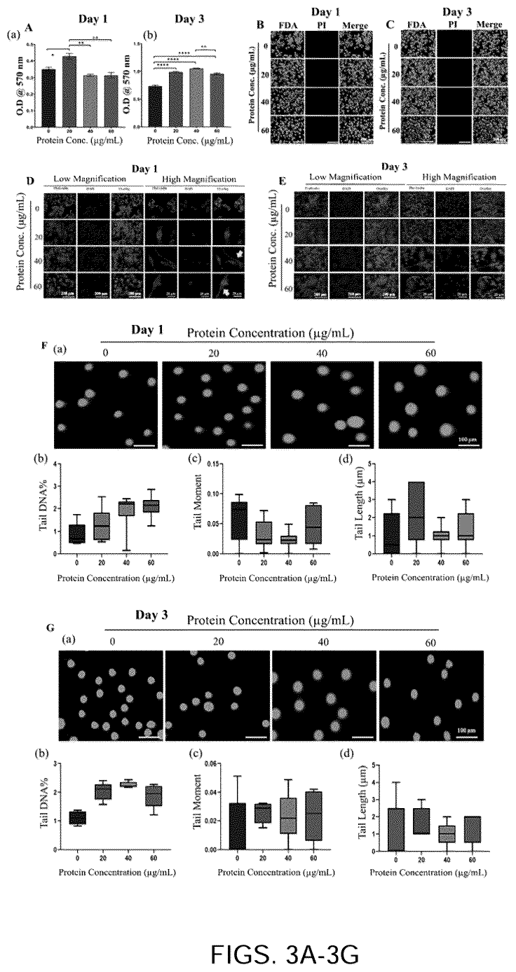

Next, and to assess host cell viability, proliferation, and cytotoxicity, a MTT assay was used to investigate the effect of LaEPs on the metabolic activity of RAW 264.7 macrophages on days 1 and 3 post-incubation (FIG. 3A). The results show that on day 1, a dose of 20 μg/mL⁻¹ of LaEPs only, significantly increased macrophage metabolic activity compared to the control group (p<0.05) and the 40, and 60 μg/mL⁻¹ groups (p<0.01 in both groups). Remarkably, on day 3, significantly increased metabolic activity was measured following treatment at all three LaEP concentrations, and when compared to the control, untreated group (p<0.0001 in all groups). Further, a live/dead assay confirmed 100% macrophage viability following supplementation with 20, 40, or 60 μg/mL⁻¹ of LaEPs on days 1 and 3 of culture (FIG. 3B). Next, and using confocal microscopy, macrophage cytoskeletal morphology via phalloidin and DAPI staining was evaluated (FIG. 3C). Remarkably, the results reveal the smaller and circular morphology of the macrophages alter where cells become much larger and predominantly spindle shaped as the LaEP dose increased on both days 1 and 3 of treatment. Whether exposure to LaEPs induced DNA damage in macrophages was then examined and the results show no significant differences when tail DNA %, tail moment, and tail length were assessed on days 1 and 3 following supplementation (FIG. 3D). Thus, the LaEPs do not induce single or double stranded DNA breaks in RAW 264.7 cells. Finally, the inflammatory effect of LaEPs on RAW 264.7 cells was analyzed using quantitative real-time reverse transcription polymerase chain reaction (qRT-PCR).

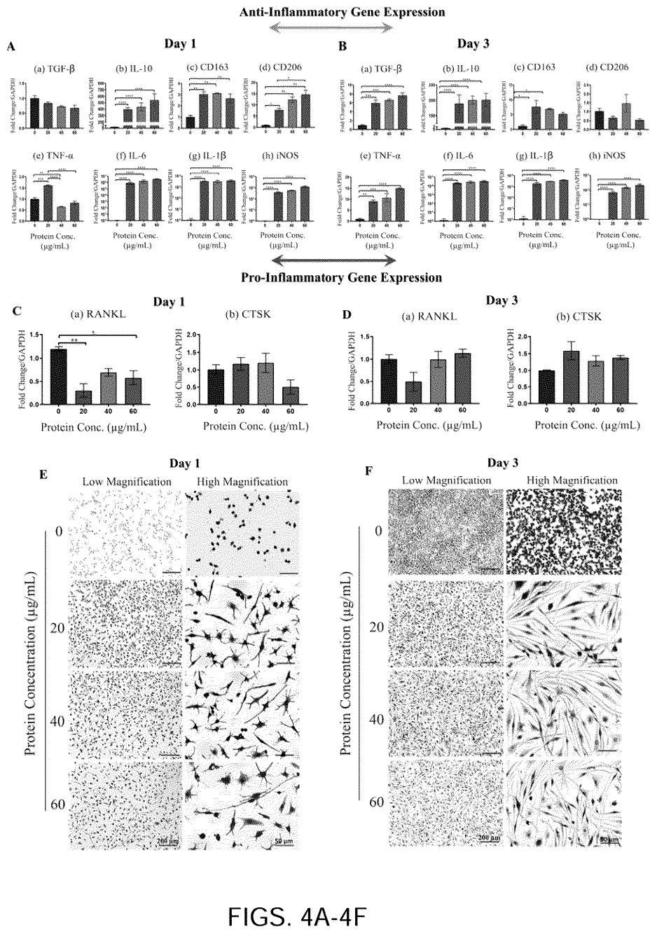

Notably, the results show a trend where LaEPs stimulated both anti-inflammatory and pro-inflammatory gene expression (FIG. 4A). On day 1, a significant and dose-dependent increase in anti-inflammatory gene expression was measured at all three LaEP concentrations. When supplemented with 60 μg/mL⁻¹ of LaEPs, IL-10 expression significantly increased ~500-fold on day 1, and ~200-fold on day 3 when compared with the control group (p<0.0001 on both days). Similarly, CD163 expression significantly increased ~2.5-fold on day 1 (p<0.01), and ~5-fold on day 3, although this increase was not significant. TGF-β expression was reduced on day 1, however, on day 3 a significant and ~7-fold increase in expression was measured (p<0.0001). Remarkably, and despite qualitative observation of a M2-like morphological phenotype, pro-inflammatory marker expression (TNF-α, IL-6, IL-1β and iNOS) also significantly increased in response to the LaEPs. IL-6 and IL-1β expression increased ~10⁶-fold, and ~10³-fold respectively on day 1, reducing to a ~10⁴-fold increase in IL-6, and maintaining a ~10³-fold increase in IL-1β expression on day 3 (p<0.0001 in all groups).

To investigate this further, and to determine whether the macrophage phenotype had differentiated towards an osteoclastic phenotype, expression of the major osteoclastogenesis marker, RANKL, and osteoclastic activation marker cathepsin K (CTSK), were quantified (FIG. 4B), together with determining the number of tartrate-resistant acid phosphatase (TRAP)-positive cells (FIG. 4C). Notably, the results show that in all of the LaEP concentrations investigated, significantly reduced RANKL expression was observed on day 1, with no significant change in CTSK expression on days 1 and 3. Further, and interestingly, a mixture of both TRAP⁺ and TRAP⁻ stained cells were identified. Here, some cells appear to display an activated macrophage phenotype with the noted presence of phagosomes within the cytoplasm. Phagocytosis begins via a complex series of steps including the formation of plasma-derived phagosomes, and subsequently phagolysosomes within which a series of antibacterial compounds are generated, and where the microbe is eventually killed, or cellular debris is digested^{74,75}. Despite this, and in support of the observed downregulation in RANKL expression, none of the cells presented with TRAP⁺ staining together with a multinucleated morphology, suggesting no evidence of osteoclastic differentiation and activity.

Isolated L. acidophilus Extracellular Proteins Downregulate Lipopolysaccharide-Induced Chronic Inflammatory Gene Expression and Protect RAW 264.7 Macrophages Against DNA Damage

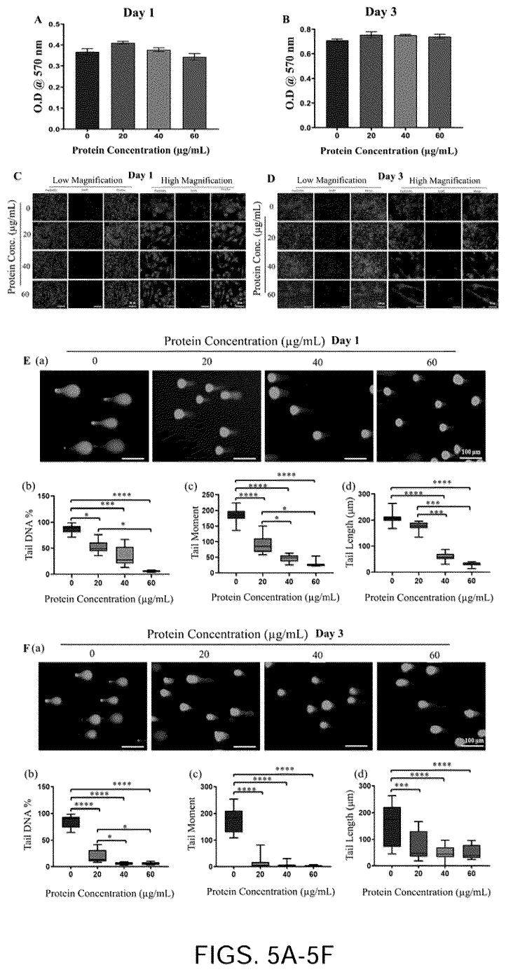

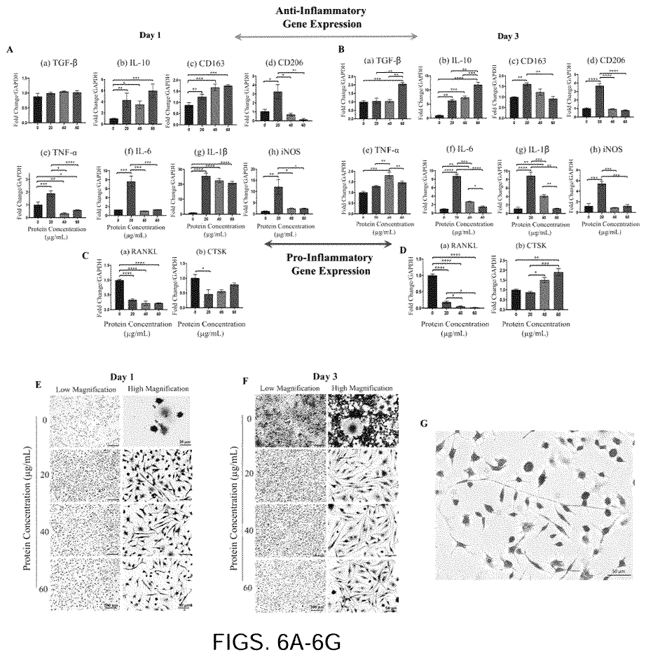

Next, whether LaEPs upregulate anti-inflammatory gene expression while under simulated chronic inflammatory conditions in vitro was investigated. RAW 264.7 macrophages were stimulated with 10 ng/mL⁻¹ of lipopolysaccharide (LPS) to induce chronic inflammatory conditions, as previously reported⁷⁶. The results show that under these culture conditions, metabolic activity increased with time however, no change in activity was measured following LaEP treatment at the 1- and 3-day timepoints (FIG. 5A). Further, cell morphological analysis on days 1 and 3 show the macrophages become more spindle-shaped when compared with control cells and display an anti-inflammatory phenotype despite the presence of LPS, and following exposure to LaEPs at all three of the concentrations investigated (FIG. 5B). Notably, and on day 3, cells in the LPS-given control group had fused to form multinucleated giant cells, a hallmark indicator of chronic inflammation. However, in comparison, cells supplemented with LaEPs remained mononucleated, spindle-shaped, and elongated. Further, the results show that LPS induced DNA damage within macrophages (FIG. 5C), and remarkably, when cells were supplemented with LaEPs, DNA damage was significantly reduced in a dose dependent manner. Notably, on day 3, the mean comet tail length measured in the control cell group with LPS, measured 159.90±26.60 μm, while cells with LPS and LaEPs at a concentration of 60 μg/mL⁻¹, demonstrated a significant, and ~8-fold reduction in mean tail length of 19.70±3.32 μm (p<0.0001).

qRT-PCR analyses on days 1 and 3 showed a heightened differential effect in both pro- and anti-inflammatory markers when LaEPs were supplemented at a concentration of 20 μg/mL⁻¹ (FIG. 6A). At this dose, IL-10 expression significantly increased ~3-fold and ~6-fold on days 1 and 3 respectively, when compared to control cells (p<0.01 in both groups). Notably, only the cells exposed to 20 μg/mL⁻¹, and not 40, or 60 μg/mL⁻¹ of LaEPs significantly increased CD206 expression ~2-fold (p<0.05), and ~2.5-fold (p<0.0001) on days 1 and 3 respectively, when compared to control. Remarkably, at this dose, and when compared to the control group, a significant increase in TNF-α (p<0.01), IL-6 (p<0.01), IL-1β (p<0.0001), and iNOS (p<0.01 was measured on day 1. This trend continued, and on day 3, significantly increased IL-6 (p<0.0001), IL-1β (p<0.0001), and iNOS (p<0.001) expression was measured in the 20 μg/mL⁻¹ LaEP group when compared to the control cell group. Notably, at the 40 and 60 μg/mL⁻¹ dose, and on day 3, IL-6, IL-1β, and iNOS expression were reduced when compared to the 20 μg/mL⁻¹ LaEP treated cells. As such, a trend was seen where the highest LaEP dose of 60 μg/mL⁻¹, displayed overall higher anti-inflammatory gene expression parallel to lower pro-inflammatory expression, suggesting that the dose of LaEPs may be significant in its role of resolving chronic inflammation in vitro. To further support this, osteoclastogenesis marker RANKL expression was significantly downregulated in all groups and in a dose-dependent manner, on days 1 and 3 (p<0.0001 in all groups) (FIG. 6B). CTSK expression reduced on day 1 and was significantly upregulated in the 40 (p<0.05), and 60 μg/mL⁻¹ (p<0.001) groups on day 3 when compared to control. Next, whether LaEP treatment prevented osteoclast differentiation and osteoclastic activity of RAW264.7 macrophages was investigated. Activated macrophage/osteoclastic activity was investigated using TRAP staining, and after 3 days, a mixture of TRAP⁺ and TRAP⁻ cells were detected in LPS stimulated RAW264.7 cells with LaEPs at all concentrations investigated (FIG. 6C).

Isolated L. acidophilus Extracellular Proteins are Non-Cytotoxic to hBMSCs

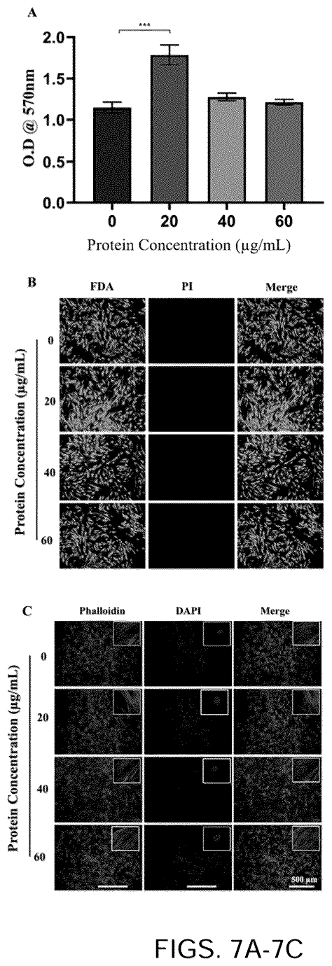

The effect of LaEPs on hBMSC metabolic activity was assessed and the results show that cells cultured in media supplemented with 20 μg/mL⁻¹ of LaEPs resulted in a significant increase in activity when compared with the control group (p<0.001, FIG. 7A). No other significant differences were found. In support of this, hBMSC viability remained high, with no dead (red) cells detected at any of the LaEP concentrations when investigated on day 3 (FIG. 7B). Further, qualitative cell morphological analysis suggested no evident changes to cytoskeletal structure and content following supplementation of hBMSCs with LaEPs at concentrations of 20, 40, or 60 μg/mL⁻¹ (FIG. 7C). Thus, and similar to RAW264.7 macrophages, LaEPs were found to be non-cytotoxic to hBMSCs at the concentrations, and 3-day timepoint investigated.

Isolated L. acidophilus Extracellular Proteins are Osteoinductive, and Promote hBMSC Osteogenic Differentiation, and Mineral Deposition

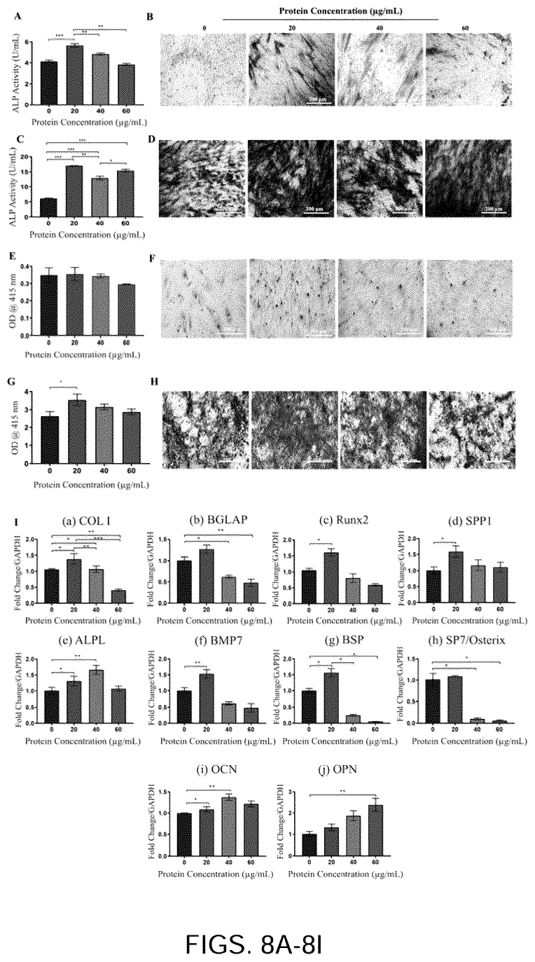

Osteoblasts and bone marrow adipocytes are derived from the same progenitor cell (i.e., BMSCs), and a switch in lineage commitment toward adipogenic differentiation at the expense of osteoblastogenesis has been identified during bacterial infection⁷⁷ and is a major contributor to various pathological bone diseases and tissue loss^{46,78,79}. Here, the effect of LaEPs on both hBMSC osteogenic and adipogenic differentiation was investigated. Osteoinduction is the process by which osteogenesis is induced and implies the direct stimulation of hBMSCs to develop into preosteoblasts⁸⁰. First, in order to determine whether LaEPs were osteoinductive, their ability to stimulate osteogenic differentiation in vitro, and in the absence of osteogenic-supplemented media (OM) was investigated. Remarkably, the results reveal that after 14 days of culture, a significant increase in ALP protein expression, an initial marker of osteoblast differentiation, was measured in both the 20 and 40 g/mL⁻¹ groups (p<0.001 and p<0.01, respectively) when compared with the control group without OM (FIGS. 8A-B). When investigated in the presence of OM, a significant ~>2-fold increase in ALP⁺ staining was measured in the 20, 40, and 60 g/mL⁻¹ treated groups compared with control (p<0.001 in all groups, FIGS. 8C-D). Notably, and following Alizarin Red staining to reveal bone mineral deposition after 28 days of culture, no mineralization was observed when hBMSCs were cultured in the absence of OM (FIGS. 8E-F). Further, and when the effect of LaEPs was investigated in the presence of OM, results showed a significant increase in mineral deposits in the 20 g/mL⁻¹ group only, and when compared with the control group (FIGS. 8G-H). The results suggest the LaEPs may provide a dose-dependent and greater regulatory role (at lower concentrations) earlier, and during lineage differentiation, with less interactions later, and when mature osteoblasts begin to deposit mineral. To support this, when hBMSCs were cultured in osteogenic media, a significant upregulation in the expression of collagen I (Col-I) (p<0.01), runt-related transcription factor 2 (Runx2) (p<0.05), secreted phosphoprotein 1 (SPP1) (p<0.01), alkaline phosphatase (ALPL) (p<0.05), bone morphogenic protein 7 (BMP-7) (p<0.01), bone sialoprotein (BSP) (p<0.05), and osteocalcin (OCN) (p<0.05) was demonstrated when LaEPs were compared to control cells at a concentration of 20 g/mL⁻¹ (FIG. 8I). Notably, osteopontin (OPN) expression increased as the LaEP dose increased, while Col-I, BSP, bone gamma-carboxyglutamate protein (BGLAP), Runx2, SPP1, BMP7, and SP7 Osterix (SP7), expression decreased with increasing concentrations of LaEPs.

Exposure to Isolated L. acidophilus Extracellular Proteins Abates hBMSC Adipogenic Differentiation and Lipid Formation

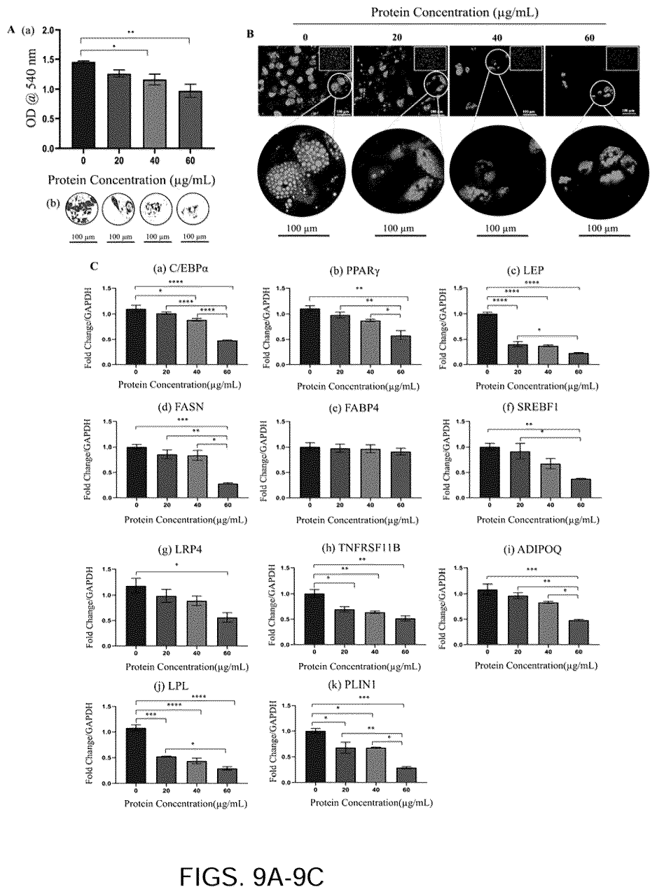

To determine whether the extracellular proteins released by L. acidophilus reduce hBMSC adipogenic differentiation, the gene expression of adipogenesis-related markers and accumulation of intracellular lipid droplets were quantified. Following hBMSC culture in adipogenic differentiation media, lipid droplet formation was observed in control cells without LaEP treatment (FIGS. 9A-C). However, when cultured with LaEPs, a dose-dependent and significant reduction in intracellular lipid formation was measured in the 40 (p<0.05), and 60 μg/mL⁻¹ (p<0.01) groups when compared with control. An increased number of control cells were observed that appeared larger in size, and with a markedly higher amount of intracellular lipid droplets when compared to LaEPs treated cells (FIG. 9B).

Next, changes in the gene expression of adipogenesis-related markers were evaluated. The following genes were investigated: CCAAT-enhancer bindingprotein α (CIEBPα), peroxisome proliferator-activated receptor gamma (PPARγ), leptin (LEP), fatty acid synthase (FASN), fatty acid-binding protein 4 (FABP4), sterol regulatory element-binding transcription factor 1 (SREBF1), low density lipoprotein receptor related protein 4 (LRP4), TNF receptor superfamily member 11b (TNFRSF11B), adiponectin (ADIPOQ), low density lipoprotein (LPL), and perilipin 1 (PLIN1). The results reveal that, except for FABP4, treatment with LaEPs significantly reduced the expression of all genes in a dose-dependent manner (FIG. 9D).

### Proteomic Profile of LaEPs Using Gel Electrophoresis and Tandem Nano-LC-MS/MS

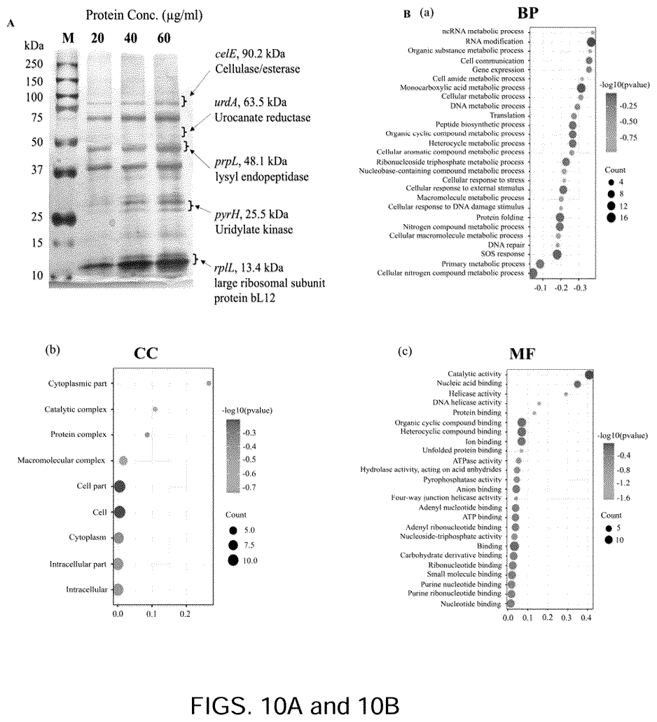

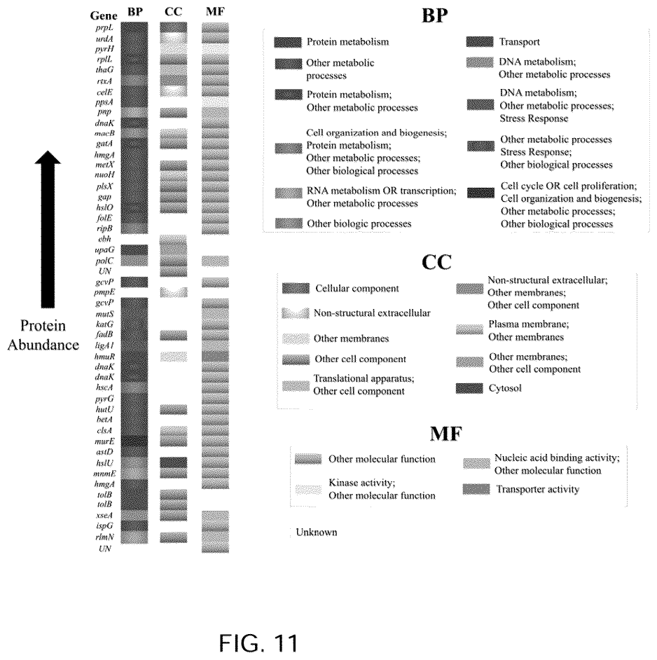

Next, the heterogeneous proteomic profile of LaEPs at a concentration of 20, 40, and 60 μg/mL⁻¹ using 1D SDS-PAGE gel electrophoresis, and at 60 μg/mL⁻¹ using nano-liquid chromatography tandem mass spectrometry (nano-LC-MS/MS) was analyzed. SDS-PAGE analyses revealed similar protein band patterns in the 20, 40, and 60 μg/mL⁻¹ concentration groups, with an expected increase in abundance observed in the 60 μg/mL⁻¹ group. Notably, a strong protein band was observed at ~10-13 kDa, with weaker intensity bands evident at ~26, 48, 75, and 90 kDa when referenced against the 10 to 250 kDa protein marker (Precision Plus Protein™ Kaleidoscope™, Biorad, USA). Further, reduced band intensities were evident almost along the entire length of the gel (FIG. 10A). The total number of putative proteins identified using nano-LC-MS/MS was 6537, with molecular weights ranging between 3.3-1156.5 kDa. Gene ontology (GO) analysis revealed the biological process (BP), cellular component (CC), and molecular functions (MF), where the top BPs were ncRNA metabolic process, RNA modification, and organic substance metabolic process. The top CCs were cytoplasmic part, catalytic complex, and protein complex, and finally, the top MFs were catalytic activity, nucleic acid binding, and helicase activity (FIGS. 10B(a-c). The top 50 measured protein abundance levels are highlighted in FIG. 11. The most abundant protein present was the lysing enzyme, lysyl endopeptidase (prpL) (Table 1), followed by urocanate reductase (urdA), uridylate kinase (pyrH), large ribosomal subunit protein bL12 (rplL), and polyketide synthase (thaG). All are putatively involved in metabolism however their specific biological and molecular functions remain elusive.

TABLE 1

| | | | | | | Score | |
|---|---|---|---|---|---|---|---|
| | | MW | | | # Unique | Sequest | |
| Abun. | Gene | (kDa) | # Accession | # PSMs | Peptides | HT | Putative Description |
| 1 | prpL | 48.1 | Q02SZ7 | 55 | 17 | 263.95 | Lysyl endopeptidase |
| 2 | urdA | 63.5 | C8WLE3 | 7 | 2 | 41.18 | Urocanate reductase |
| 3 | pyrH | 25.5 | A1K6R9 | 5 | 1 | 5.27 | Uridylate kinase |
| 4 | rplL | 13.4 | B7ICR4 | 5 | 2 | 16.01 | Large ribosomal subunit protein |
| | | | | | | | bL12 |
| 5 | thaG | 587 | Q2T4N1 | 4 | 4 | 18.19 | Polyketide synthase ThaG |
| 6 | rtxA | 485.1 | Q9KS12 | 4 | 4 | 12.7 | Multifunctional-autoprocessing |
| | | | | | | | repeats-in-toxin |
| 7 | celE | 90.2 | P10477 | 4 | 1 | 8.48 | Cellulase/esterase CelE |
| 8 | ppsA | 87.4 | P23538 | 4 | 1 | 10.04 | Phosphoenolpyruvate synthase |
| 9 | pnp | 77.2 | B4RC48 | 4 | 1 | 16.24 | Polyribonucleotide |
| | | | | | | | nucleotidyltransferase |
| 10 | dnaK | 68.7 | Q2RNE6 | 4 | 2 | 14.22 | Chaperone protein DnaK |
| 11 | macB | 67.7 | Q5P6D5 | 4 | 1 | 8.59 | Macrolide export ATP- |
| | | | | | | | binding/permease protein MacB |
| 12 | gatA | 51.9 | C5BSY8 | 4 | 2 | 11.88 | Glutamyl-tRNA(Gln) |
| | | | | | | | amidotransferase subunit A |
| 13 | hmgA | 50.1 | A3MMH2 | 4 | 1 | 12.58 | Homogentisate 1,2-dioxygenase |
| 14 | metX | 43.1 | Q8PK44 | 4 | 1 | 18.31 | Serine O-succinyltransferase |
| 15 | nuoH | 40.1 | Q8A0F7 | 4 | 1 | 3.47 | NADH-quinone oxidoreductase |
| | | | | | | | subunit H |
| 16 | plsX | 37.4 | Q1QMZ1 | 4 | 2 | 5.73 | Phosphate acyltransferase |
| 17 | gap | 36.5 | O32755 | 4 | 3 | 15.15 | Glyceraldehyde-3-phosphate |
| | | | | | | | dehydrogenase |
| 18 | hslO | 31.4 | Q5WLV4 | 4 | | 10.65 | 33 kDa chaperonin |
| 19 | folE | 24.8 | P0A6T6 | 4 | 1 | 16.49 | GTP cyclohydrolase 1 |
| 20 | ripB | 24.6 | P9WHU5 | 4 | 4 | 24.64 | Peptidoglycan endopeptidase RipB |
| 21 | ebh | 1144.2 | A5ISW6 | 3 | 1 | 13.45 | Extracellular matrix-binding |
| | | | | | | | protein ebh |
| 22 | upaG | 177.6 | A0A0H2VCA1 | 3 | 2 | 9.94 | Autotransporter adhesin UpaG |
| 23 | polC | 161.4 | Q038M0 | 3 | 3 | 11.99 | DNA polymerase III PolC-type |
| 24 | UN | 134.4 | B2RHG5 | 3 | 1 | 14.49 | Minor fimbrium subunit Mfa5 |
| 25 | gcvP | 104.8 | Q6LHN5 | 3 | 3 | 17.18 | Glycine dehydrogenase |
| | | | | | | | (decarboxylating) |
| 26 | pmpE | 104.6 | O84877 | 3 | 2 | 13.66 | Probable outer membrane protein |
| | | | | | | | PmpE |
| 27 | gcvP | 100.8 | A4TA90 | 3 | 3 | 6.87 | Glycine dehydrogenase |
| | | | | | | | (decarboxylating) |
| 28 | mutS | 95.7 | B0T142 | 3 | 2 | 7.25 | DNA mismatch repair protein |
| | | | | | | | MutS |
| 29 | katG | 79.6 | A4T0J1 | 3 | 1 | 14.04 | Catalase-peroxidase |
| 30 | fadB | 77.9 | Q6FF68 | 3 | 1 | 4.53 | Fatty acid oxidation complex |
| | | | | | | | subunit alpha |
| 31 | ligA1 | 75 | B1VUC2 | 3 | 2 | 18.16 | DNA ligase 1 |
| 32 | hmuR | 74.2 | Q56989 | 3 | 1 | 10.47 | Hemin receptor |
| 33 | dnaK | 69.8 | A4JBS1 | 3 | 2 | 13.98 | Chaperone protein DnaK |
| 34 | dnaK | 69.3 | Q47HK2 | 3 | 2 | 14.41 | Chaperone protein DnaK |
| 35 | hscA | 65.8 | A4WDA7 | 3 | 2 | 11.21 | Chaperone protein HscA |
| 36 | pyrG | 63.6 | AOPPA8 | 3 | 1 | 15.8 | CTP synthase |
| 37 | hutU | 61.5 | B2K6P6 | 3 | 1 | 9.5 | Urocanate hydratase |
| 38 | betA | 61.4 | Q6FDF9 | 3 | 2 | 13.13 | Oxygen-dependent choline |
| | | | | | | | dehydrogenase |
| 39 | clsA | 54.9 | B7LS29 | 3 | 3 | 8.39 | Cardiolipin synthase A |
| 40 | murE | 52.4 | Q87AF5 | 3 | 2 | 2.75 | UDP-N-acetylmuramoyl-L-alanyl- |
| | | | | | | | D-glutamate-2,6-diaminopimelate |
| | | | | | | | ligase |
| 41 | astD | 51.8 | A9AD19 | 3 | 1 | 16.52 | N-succinylglutamate 5- |
| | | | | | | | semialdehyde dehydrogenase |
| 42 | hslU | 50.1 | A6VDH5 | 3 | 2 | 11.19 | ATP-dependent protease ATPase |
| | | | | | | | subunit HslU |
| 43 | mnmE | 49.8 | Q6LW56 | 3 | 2 | 4.79 | tRNA modification GTPase MnmE |
| 44 | hmgA | 48.9 | A0K4Z6 | 3 | 1 | 11.27 | Homogentisate 1,2-dioxygenase |
| 45 | tolB | 48.1 | Q726K4 | 3 | 3 | 8.68 | Tol-Pal system protein TolB |
| 46 | tolB | 47.1 | B2STJ5 | 3 | 1 | 17.64 | Tol-Pal system protein TolB |
| 47 | xseA | 47 | B5ZA25 | 3 | 2 | 8.66 | Exodeoxyribonuclease 7 large |
| | | | | | | | subunit |
| 48 | ispG | 46.2 | Q5FHA6 | 3 | 1 | 14.57 | 4-hydroxy-3-methylbut-2-en-1-yl |
| | | | | | | | diphosphate synthase (flavodoxin) |
| 49 | rlmN | 45.7 | A3M208 | 3 | 2 | 10.29 | Dual-specificity RNA |
| | | | | | | | methyltransferase RlmN |
| 50 | UN | 45.6 | P0DO96 | 3 | 2 | 9.65 | Citrate (Re)-synthase |

Table 1 lists descriptions of LaEP (60 μg/mL¹) peptides measured using nano-LC-MS/MS. Accession numbers are from the nrNCBI database. The identity of the peptides/proteins was confirmed by the sequencing of the MS/MS data followed by Sequest HT searches, a tandem mass spectrometry database search program. A total of 6537 master proteins with molecular weights ranging between 3.3-1156.5 kDa were identified. The top 50 most abundant (PSM) peptides and a description of putative functions are described. Abun.=abundance, MW=molecular weight, AA=amino acid, PSM=peptide spectrum match, UN=unknown.

### Discussion

The aim of Experimental Example 1 was to investigate L. acidophilus-S. aureus co-aggregation and pathogenic growth inhibition together with a primary focus on the effect of isolated extracellular-derived L. acidophilus proteins on PJI relevant S. aureus, MRSA, and E. coli planktonic growth and biofilm formation. Further, and in parallel, Experimental Example 1 aimed to investigate the effect of LaEPs on host macrophages and osteogenic, and adipogenic hBMSC differentiation in vitro. The results reveal that LaEPs confer bacteriostatic properties, prevent biofilm formation, beneficially regulate chronic inflammation, promote a reparative macrophage phenotype, and augment bone deposition while abating adipogenesis.

Although widespread, bacterial auto-aggregation is poorly understood. Haaber et al⁸¹, demonstrated that cell aggregates of S. aureus display a mean 7-fold increase in metabolic activity when compared to planktonic cells. Thus, auto-aggregation is considered to provide pathogenic bacteria with the ability to maintain mobility whilst also evading host defenses and antimicrobial treatment. However, co-aggregation with probiotic bacteria may neutralize pathogenic strains, preventing their motility and adhesion to host cells, as well as the mucosal surface thereby creating a barrier to colonization within the hosts^{82,83}. If the Lactobacilli adhesion forces with pathogens are stronger than those binding the pathogen together, then this may describe their disruptive properties. In terms of S. aureus, our ζ potential results reveal L. acidophilus species were more anionic and the % aggregation was immediately, and significantly higher in the L. acidophilus-S. aureus co-cultured group when compared with a monoculture of each bacterial species alone. Although these forces were not quantified in Experimental Example 1, the electrostatic L. acidophilus-S. aureus interactions were likely stronger than the S. aureus-S. aureus interactions⁶². Further, the data show increased % aggregation but with a smaller aggregate size compared to monocultured S. aureus at the earlier timepoints investigated. Notably, and over a longer duration, the S. aureus aggregate size decreased compared to the L. acidophilus-S. aureus group. This disparity may be accounted for by preferential L. acidophilus-S. aureus bond-strengthening, where adhesion forces become stronger over time due to the progressive removal of water in between the interacting cell surfaces, rearrangement of surface structures, and the unfolding of binding molecules⁶². Further, and notably during co-culture, the TEM imaging revealed multiple S. aureus cocci adhered to single L. acidophilus cells. Nevertheless, and similar to other studies^{55,84,85}. L. acidophilus growth was maintained and a significant 3-log reduction in S. aureus growth was demonstrated. Lactobacilli show great diversity in cell surface architecture and can modify their surface properties in response to environmental changes⁸⁶⁻⁸⁷. To this end, and in addition to electrostatic forces, or physical interactions such as van der Waals forces⁸⁸, surface-associated Lactobacilli molecules, including lipoteichoic acid, sortase-dependent proteins⁸⁹⁻⁹⁰, aggregation promoting factor⁹¹, surface layer A protein (SlpA)⁹², lipoproteins, and a myosin-cross-reactive protein⁹³, have been associated with its probiotic co-aggregation attributes. While the release of antimicrobial compounds, and nutrient competition from L. acidophilus will undoubtedly play a key role in the inhibition of S. aureus growth observed, further work to investigate the contribution of direct physical interaction and binding between the microbes and abated cell growth is warranted. This is of relevance as probiotic adherence to host cells and tissue, or metal implant surfaces will theoretically limit sites available for pathogenic adherence thereby reducing the incidence of pathogenic infection⁹⁴⁹⁵. However, Lactobacillus spp. have been associated with PJI^{69,70}. Thus, an improved understanding of the adhesion forces at the micro- and nano-level will undoubtedly be critical to the development of novel therapeutic interventions that target the co-aggregation of probiotic-pathogenic species and/or expedite advantageous, and prompt probiotic implant and tissue surface occupation to prevent pathogenic PJI adhesion and colonization.

While studies have demonstrated the inhibition of S. aureus, E. coli, and MRSA biofilm formation and its disassembly, and E. coli activity and adherence is reduced when exposed to Lactobacillus CFS, this response has primarily been attributed to acidification of the culture media by the fermentation of lactic acid, acetic acid, and production of hydrogen peroxide (H₂O₂), among other biomolecules^{56,96-99}. As the exogenous introduction of e.g., lactic acid, acetic acid, and H₂O₂ as a therapeutic approach to patients is improbable, here, L. acidophilus proteins were isolated and their direct effect on pathogenic biofilm formation was investigated. Proteins derived from L. acidophilus exhibit promising characteristics in terms of inhibiting the formation of pathogenic biofilmss⁵². The results show LaEPs significantly reduced biofilm formation and thickness in a dose-dependent manner in all three of the bacterial species investigated. Notably, and while the LaEPs reduced biofilm formation, the results also reveal that cells remained viable following treatment, suggesting that at the dose and timepoints investigated, LaEPs are bacteriostatic to E. co/i, S. aureus, and MRSA. In support of this, Ong et al¹⁰⁰. investigated the protein-rich fraction isolated from L. fermentum and demonstrated significantly reduced S. aureus growth and biofilm thickness. Interestingly, the proteinaceous metabolites inhibited staphyloxanthin, a golden carotenoid pigment, which serves as a reactive oxygen radical scavenger, protecting proteins and DNA from oxidative stress. Further the cationic proteins inhibited quorum-sensing via electrostatic adhesion to the negatively charged S. aureus membrane. Although not S. aureus, Jung et al¹⁰¹. demonstrated that the lipoteichoic acids of Lactobacilli inhibited Enterococcus faecalis biofilm formation and disrupted preformed biofilm on the surface of dentin slices, however, pathogenic growth remained unaffected. Kim et al¹⁰². reported bacteriocins produced by Lactobacillus brevis significantly reduced E. coli biofilm formation, while Cisneros et al¹⁰³. showed that Lactobacilli species were able to reduce preformed E. coli biofilm as well as exert a Lactobacilli-induced growth inhibitory effect. Investigation of the direct effect of the protein-rich fractions or lipoteichoic acids produced by Lactobacillus strains on S. aureus, MRSA, and E. coli are limited. However, the findings, together with the studies described, suggest an impactful contribution and further work is warranted to uncover their mechanistic contributions and any conceivable therapeutic role in the prevention and treatment of PJI.

Although activated macrophages play a pivotal role in both initiating and maintaining chronic inflammation, their deactivation is also central to resolving inflammation and in promoting bone regeneration and repair^{104,105}. As such, and depending on their polarization, macrophages contribute an essential osteoprotective or osteodestructive response. Further, and to ensure optimal host health, macrophages deliver major phagocytic activity, and are specialized to detect, engulf, and destroy some bacteria and viruses¹⁰⁶. Thus, their concerted activity is pivotal to the post-surgical host response following e.g., TKA, and THA, and their subsequent expedient contribution to bone repair, the host cell-microbial ‘race to the surface’, and ultimately, implant osseointegration. In Experimental Example 1, the effect of LaEPs on macrophage activity was investigated under both ambient and simulated chronic inflammatory culture conditions. The data show that when cultured under typical ambient conditions, no reduction in macrophage viability was identified and the non-cytotoxic LaEPs at all concentrations failed to induce DNA damage. Remarkably, a trend was seen where the higher the LaEP dose, the higher the simultaneous expression of both pro- and anti-inflammatory genes. Activated M1 (osteoclastic) macrophages are typically small and round, and can be deactivated towards a reparative, elongated, and spindle shaped M2 phenotype^{107,108} via anti-inflammatory cytokines including IL-10, TGF-β, CD163, and CD206, which are mainly produced by macrophages^{104,109}. Further, RANKL promotes osteoclastogenesis, and CTSK is a secreted protease that plays an essential role in osteoclastic bone resorption¹¹⁰, and is expressed by activated osteoclasts^{111,112} and is considered one of the most potent collagenases¹¹³. During osteoclastic activation, both RANKL and CTSK are typically upregulated¹¹⁴. To distinguish between the M1, and M2/osteoclastic phenotype, macrophage morphology, the expression of RANKL and CTSK, and the presence of TRAP⁺ staining was assessed. The results show the cells remained mononucleated, with reduced levels of RANKL and CTSK on day 1 and became elongated and spindle shaped in a dose- and time-dependent manner. This suggests LaEP-induced differentiation towards the pro-healing M2 phenotype. However, and paradoxically, many cells were TRAP⁺, thereby also confirming the presence of activated M1 macrophages within the culture. Further, and when investigated under LPS-induced chronic inflammatory conditions, a notable trend was seen where the higher the concentration of LaEPs, the higher the upregulation of anti-inflammatory expression, together with a reduction in pro-inflammatory gene expression. This was also accompanied by a significant reduction in RANKL expression on days 1 and 3, suggesting limited osteoclastogenic activity, although parallel to extensive TRAP⁺ staining. Notably, the data shows CTSK expression remained either similar to control samples or increased in response to LaEPs.

Firstly, TRAP is a member of a heterogeneous group of lysosomal enzymes of which there are two forms, namely, TRAP5a and TRAP5b¹¹⁵. These two isoforms are structurally and antigenically identical, however, TRAP5a is produced by activated macrophages, and TRAP5b is secreted by osteoclasts¹¹⁶. The expression of TRAP5a versus TRAP5b was not quantified in this experiment, however, and based on the extensive presence of cytoplasmic phagosomes, upregulated anti-inflammatory expression, together with significantly reduced RANKL, it was speculated that the TRAP⁺ staining observed in this experiment was primarily TRAP5a in nature, thus indicating the presence of activated macrophages (and thus IL-1β, IL-6, JNFα, and INOS) and not osteoclasts. Secondly, the phagocytic activity of macrophages occurs via the presence of a repertoire of cathepsins¹¹⁷, where pathogenic bacteria are reported capable of influencing cathepsin expression and proteolytic activity to favor their own intracellular survival¹⁰⁶. A recent proteomic study reported CTSK as a crucial molecular factor required for macrophage phagosome formation, in preparation for intracellular protein and collagen degradation via phagocytosis¹¹⁸. Experimental Example 1 showed that pharmacological blockage of CTSK activity diminished phagosomal proteolysis and collagenolysis in lung-resident macrophages, contributing to reduced phagocytic collagen clearance in lungs. As macrophages are the first encounters of invading bacteria, this activity is also highly relevant to bacteria-macrophage interaction and subsequent sentinel microbial elimination¹⁰⁶. Regulating phagocytic macrophage activity by probiotics has been shown to enhance their ability to engulf and kill pathogenic bacteria, thereby augmenting the innate host immune response to infection¹¹⁹. However, the mechanisms which impact macrophage phagocytic capacity and downstream phagosomal processing remain poorly understood. The phagocytic response to S. aureus was not investigated in this experiment and as a dose-dependent response was measured, further investigation is essential to determine the role of LaEP-induced enhanced phagocytic bacterial clearance. In support of this and using conditioned medium obtained from cultures of L. rhamnosus, Nanjundaiah et al¹¹⁹. showed a dose-dependent increase in the bacterial killing ability of macrophages against E. coli, and that this was found to be associated with the enhanced production of reactive oxygen species (ROS) and nitric oxide (NO) in differential amounts, within macrophages. Similarly, Rocha-Ramirez et al¹²⁰. demonstrated that the pretreatment of macrophages with varying Lactobacillus strains increased their phagocytic and bactericidal activity towards pathogens including S. aureus and E. coli. However, the results contrast with a previous study by Hidalgo-Cantabrana et al.¹²¹, which confirmed that daily administration of extracellular proteins, mainly composed of SlpA, and isolated from L. acidophilus, decreased the production of peripheral blood mononuclear cell-induced pro-inflammatory cytokines in both healthy and Crohn's disease patients with and without LPS. Further, the strong and dose-dependent anti-inflammatory properties of L. acidophilus spp. have been reported¹²²⁻¹²⁶, where combined probiotics showed the most efficacious anti-inflammatory activity^{121,127}, potentially via NF-κB signaling¹²⁸.

Lipopolysaccharide (LPS) stimulates the synthesis of substantial levels of NO, ROS, and cyclooxygenase-2 (COX-2) in macrophages resulting in cellular oxidative stress¹²⁹⁻¹³¹, and DNA damage¹³². In support of this, although NO, ROS, and COX-2 were not investigated in this experiment, the results demonstrated a substantial increase in LPS-induced single- and double-strand DNA damage where notably, supplementation with LaEPs significantly reduced DNA damage; decreasing damage as the LaEP dose increased. Production of NO and ROS is an effective tool for eliminating microbes. However, it does put the survival of monocytes and macrophages at risk, blocking the tissue reconstruction phase, which can lead to chronic inflammation¹³³⁻¹³⁵. The subsequent macrophage response to DNA damage can determine its differentiation phenotype to for example, inflammatory dendritic cells¹³⁵, and DNA strand breaks in macrophages play a key role in immune senescence¹³⁶. To this end, further work is required to investigate the beneficial role of LaEPs in regulating the DNA damage-induced inflammatory/reparative process in macrophages, and in response to invading pathogenic bacteria.

The results show that under both ambient and chronic inflammatory conditions in vitro, LaEPs activated macrophages, priming them for phagocytosis, as well as induced a reparative, anti-inflammatory M2 macrophage phenotype. These antimicrobial and pro-repair properties are likely highly beneficial following orthopaedic surgery. The category of M2 polarized macrophages can be further subdivided¹³⁷, however generally, the M2 phenotype produces many osteogenic growth factors including bone morphogenetic protein 2, osteopontin (OPN), and 1,25-dihydroxy-vitamin D3, and also promotes BMSC osteogenic differentiation, and bone formationios,¹³⁸⁻¹⁴¹. Several studies have shown the significant, and beneficial effect of L. acidophilus spp. on promoting bone formation, preventing bone loss, and in ameliorating osteoporosis^{125,142-145}. The results further support these studies as LaEPs at the lower concentration of 20 μg/mL⁻¹ significantly upregulated hBMSC viability, ALP activity, and bone mineral deposition when compared with control cultures. Alkaline phosphatase has been shown to be a sensitive and reliable indicator of bone metabolism¹⁴⁶, with a role in osteoid formation, and in increasing inorganic phosphate local rates to facilitate mineralization^{147,148}. Runx2 and Osterix (SPP7) are both required for early and late stages of osteoblast differentiation^{149,150}, where Runx2 is a master regulator for inducing osteoblast differentiation. Notably, a trend was seen where ALP, Runx2, and SPP7 together with COLI, BGLAP, SPP1, ALP, BMP7, BSP, OCN, and OPN were upregulated primarily when supplemented at a LaEP concentration of 20 μg/mL-1. Notably, the osteoinductive properties of LaEPs were investigated and remarkably, results showed significantly increased ALP activity, and thus osteoblastogenesis, at a concentration of 20 μg/mL⁻¹ and when in the absence of OM media. However, no increase in mineral deposition was measured. Further work is needed to clarify its ability to selectively osteoinduce dose-dependent osteoblastogenesis and not mineral formation.

Bone marrow adipocytes are derived from the same progenitor cells as osteoblasts (i.e., BMSCs). PPARγ inhibits the Runx2-mediated transcription of OCN in osteoblasts and terminal osteoblast differentiation¹⁵¹, and a switch in lineage commitment toward osteogenic differentiation at the expense of adipogenesis is considered a promising approach for bone regeneration and repair. To this end, the results show LaEPs upregulate the expression of several osteogenic genes in hBMSCs, while downregulating many adipogenesis markers, and lipid accumulation primarily when administered at the higher dose of 40, and 60 μg/mL⁻¹. No other studies have reported the effect of extracellular proteins isolated from Lactobacillus spp. on adipogenesis. However, Kang et al¹⁵². found that L. acidophilus spp. when administered to mice fed a high-fat diet (IFD), reduced body weight, fat mass, inflammation, and insulin resistance, with improvements in glucose and lipid metabolism. Similarly, Park et al¹⁵³. showed that L. acidophilus significantly reduced obesity in HFD-fed mice, while Kim et al¹⁵⁴. reported an anti-adipogenic effect of L. fermentum spp. on preadipocytes via the AMPK pathway. L. plantarum has also been described to confer an anti-adipogenic effect¹⁵⁵. However, the molecular mechanisms remain unclear.

Finally, the proteomic analyses reveal a highly heterogenous LaEP population composed of 6537 putative proteins. The data highlights high abundance of lysyl endopeptidase, urocanate reductase, uridylate kinase, large ribosomal subunit protein bL12, and polyketide synthase. Notably, and although not L. acidophilus, ribosomal protein L12 has been found to be associated with the outer surfaces of L. plantarum and L. lactis, and is reported as a protective antigen against pathogens, and one able to positively regulate the host immune system^{156,157}. Further chaperone protein DnaK was also abundant in our analysis, and previous studies have hypothesized a role in the immunomodulatory, and inflammatory resolving properties of L. plantarum^{158,159}. Glyceraldehyde-3-phosphate dehydrogenase was also found in comparatively high abundance, and this otherwise intracellularly located enzyme has been identified on the surface of a variety of Lactobacillus strains. Its presence is reported to be associated with binding to the lipoteichoic acid on the pathogen cell surface, where it acts as a virulence-associated immunomodulatory protein, competing with pathogens that infect the intestinal muscosa¹⁶⁰. Bacteriocins are a very heterogeneous group composed of small peptides consisting of 19 to 37 amino acids with molecular weights largely but not limited to ~10-13 kDa¹⁶¹. Notably, and although strong intensity bands were identified at MWs ~10-13 kDa via SDS-PAGE, no putative bacteriocins, e.g., lactacin B, F, and acidocin were identified using nano-LS-MS/MS. It is nevertheless anticipated that the peptides located within these bands likely include unidentified bacteriocins and antimicrobial peptides, and further work is needed to confirm this. It is speculated that either individual proteins or those able to contribute when combined i.e., via a common molecular pathway or potentially through synergistic activity, afforded the ability of LaEPs to deliver distinct properties to both pathogens and host cells. The precise activities of many of the proteins identified remain unresolved, and further work is needed to elucidate their specific biological and molecular function/s as well as the subsequent isolation of the active agents responsible. In turn, this will allow for a more focused approach towards augmenting LaEP-induced bacteriostatic activity and the promotion of a pro-osteogenic, and reparative inflammatory phenotype.

### Conclusion of Experimental Example 1

The in vitro data suggest LaEPs hold promise as a potential bacteriostatic therapeutic agent able to reduce S. aureus biofilm formation. Further, LaEPs are non-cytotoxic to RAW264.7 macrophages and hBMSCs, induce the activation of macrophages priming them for enhanced microbial phagocytosis via CTSK upregulation, reduce LPS-induced DNA damage and RANKL expression, increase anti-inflammatory gene expression, and promote a reparative M2 morphology when under chronic inflammatory conditions. The data also shows LaEPs significantly augment osteogenesis while downregulating adipogenesis and lipid formation. Notably, a trend was observed where the higher doses of LaEPs were more effective in preventing pathogenic biofilm formation, reducing LPS-induced DNA damage, increasing overall anti-inflammatory gene expression parallel to pro-inflammatory expression, CTSK expression, and in reducing adipogenesis and lipid accumulation. A lower dose was more effective at promoting cell metabolic activity, osteogenesis, and mineralization. Lactic acid bacteria qualify for presumption of safety status and are thus considered safe by the U.S. Food and Drug Administration¹⁶²⁻¹⁶⁴. As such, LaEPs hold promise as a future clinical approach potentially able to reduce biofilm formation, while priming the host immune defense system to augment microbial phagocytosis and reparative inflammatory resolution while in parallel, augmenting new bone growth. Potential applications include LaEP coatings applied pre-operatively to the implant surface, or a gel or spray solution applied directly to the implant and adjacent bone surfaces peri-operatively. Finally, the human gastrointestinal tract harbors a complex and dynamic collection of bacteria, archaea, and eukarya, which all cross-interact to form a mechanistically sophisticated ecosystem. This study investigated a single strain, and notably one of 261 classified Lactobacillus spp¹⁶⁵. To this end, the secretome characteristics of different Lactobacilli are distinct¹⁶⁶, and therefore their capacity to crosstalk not only with each other, but other phyla will also undoubtedly influence the varying factors encountered within host bone tissue and on an abiotic implant surface. As such, further work may be beneficial to analyze varying Lactobacilli exoproteomes and their crosstalk, as this may aid in identifying candidate effector molecules, and thus, potentially advance the future development of a multimodal strategy to improve the prevention and outcome of PJIs.

### Experimental Example 2—LaEPs/CNPs: Cerium Oxide Nanoparticles (CNPs) Coated with Proteins Isolated from Gut Probiotic Lactobacillus acidophilus (LaEPs)

### Introduction

This experiment shows that when the cerium oxide nanoparticles (CNPs) are coated with proteins isolated from gut probiotic Lactobacillus acidophilus (LaEPs), a synergistic effect to cells is seen where: 1) an increase in cell metabolism occurs, this means that there is an increase in cell viability and a propensity to proliferate and increase their numbers, which indicates a likely beneficial response to new tissue formation; 2) the coated LaEPs/CNPs up and downregulate certain genes that indicate they are better able to regulate a harmful inflammatory response compared to when either CNPs or LaEPs are given alone; and 3) when the coated LaEPs/CNPs are given to the cells also in the presence of unbound LaEPs, a further different and beneficial synergistic effect is seen.

It is noted that the terms “precoated CNPs” and “coated CNPs” are used interchangeably herein. The “pre” generally refers to coating of the CNPs prior to use/administration. LaEPs/CNPs can be referred to as both or either of the terms “precoated CNPs” and “coated CNPs”.

The cells will recognize the proteins on the CNP surface, and not the CNP itself, and these results suggest that the LaEPs/CNPs may enter the cell more efficiently or via a different mechanism when the CNPs are given alone. The presence of unbound LaEPs may trigger the cell (independently) to further increase nanoparticle uptake. Or the uptake of coated LaEPs/CNPs may not increase, but once inside the cells the proteins detach exposing the CNP surface, and both may trigger an improved cellular response (individually or in combination).

Many researchers have attempted coating nanoparticles with proteins, but one aspect of the present invention is coating the CNPs with proteins isolated from the gut microbiome and the exhibited need for unbound LaEPs to achieve enhanced results. Due to the differing surface kinetics, it is likely that certain proteins will bind to the cerium oxide CeONP^{60/40} formulation, and others to the cerium oxide CeONP^{70/30} formulation, thus expanding areas of potential use.

Presently, no technology has been developed to utilize probiotics isolated from microbiomes in this way. No material-based strategy has been shown to enhance the efficacy of these probiotic proteins. The instant inventors are first to confirm their synergistic interaction with ceria (CNPs). There is much research interest in finding a drug/therapy that can reduce pathogenic bacteria while also boosting surrounding host tissue, but to date, no effective approach has been discovered, and as such, this invention offers advantages over other existing techniques and technologies.

Because the microbiome is involved in the maintenance and repair of many tissues and organs in the body, the formulations could be tuned and repurposed for potentially any area of the body. For example, gut microbiomes have been shown to beneficially affect, inter alia, osteoarthritis, osteoporosis, Alzheimer's disease, and immune disorders. The inventive formulations disclosed herein can potentially be used in solutions as an injectable, or as an implant coating for insertion into the body, or as a topical formulation/cream to assist with wound healing.

### Macrophage Metabolic Activity

FIG. 12* shows the effect of LaEPs-CNPs (cerium oxide nanoparticles (CNPs) coated with extracellular proteins isolated from gut probiotic Lactobacillus acidophilus (LaEPs) on macrophage metabolic activity. Cell metabolic activity was measured and the effect of coating cerium oxide nanoparticles (CNPs) with extracellular proteins isolated from the gut probiotic Lactobacillus acidophilus (LaEPs) was investigated. Data shows that coated CNPs at a LaEP concentration of 40 μg/mL (orange) produced significantly increased cell activity when compared with a 40 μg/mL solution of LaEPs (red) or CNPs (yellow) alone. This shows that a synergistic effect was delivered. A further and enhanced effect was seen when the coated CNPs were given to the cells in the presence of unbound LaEPs within the solution (blue). The reason for this enhancement is unclear but may involve the LaEPs stimulating the cells to increase their uptake of the coated CNPs, thereby further increasing cell activity. LaEPs delivered dose-dependent differences.

FIG. 13* shows the effect** of LaEPs-CNPs on amounts of the pro-inflammatory factor interleukin-1β (IL-1β). Here, the macrophages have been treated with lipopolysaccharide (LPS) to simulate a chronic inflammatory condition akin to that seen within the body. IL-10 is a pro-inflammatory factor, but previous work of the instant inventors has shown that high levels of IL-1β do not necessarily result in an overall damaging effect. Nevertheless, the data shows that LaEPs/CNPs alongside the presence of unbound LaEPs results in a synergistic and substantial increase in this molecule.

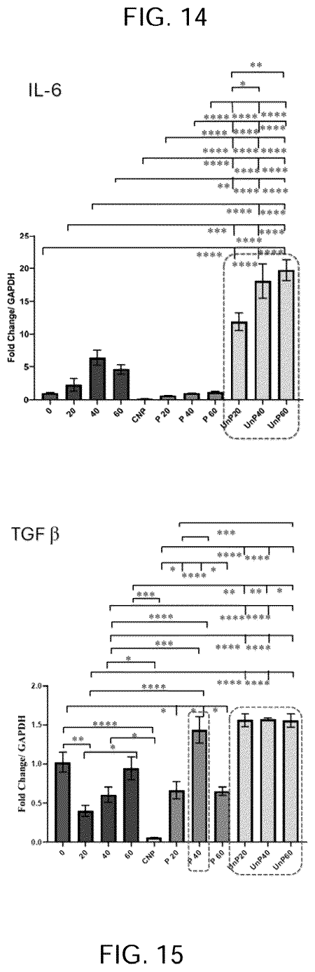

FIG. 14* shows the effect** of LaEPs-CNPs on amounts of the pro-inflammatory factor interleukin-6 (IL-6). Under simulated harmful chronic inflammatory conditions IL-6 (pro-inflammatory factor) is significantly elevated in the LaEPs/CNPs (with unbound LaEPs groups). Although a pro-inflammatory factor, high levels have previously not necessarily resulted in pro-tissue destructive cell activity. This data is included to highlight the substantial synergistic effect that occurs when coated CNPs with LaEPs together with the presence of unbound LaEPs has on altering/regulating the generation of IL-6.

FIG. 15* shows the effect** of LaEPs-CNPs on amounts of the pro-healing factor transforming growth factor β (TGFβ). Under simulated harmful chronic inflammatory conditions TGFb (pro-healing factor) is significantly elevated in the LaEPs/CNPs (with unbound LaEPs) groups and in the CNP group coating with a 40 μg/mL solution of LaEPs. This result is similar to results of FIG. 12 and suggests that CNPs coated with a 40 μg/mL solution of LaEPs produces a synergistic effect, as well as the coated CNPs at all LaEP concentrations when in the presence of unbound LaEPs.

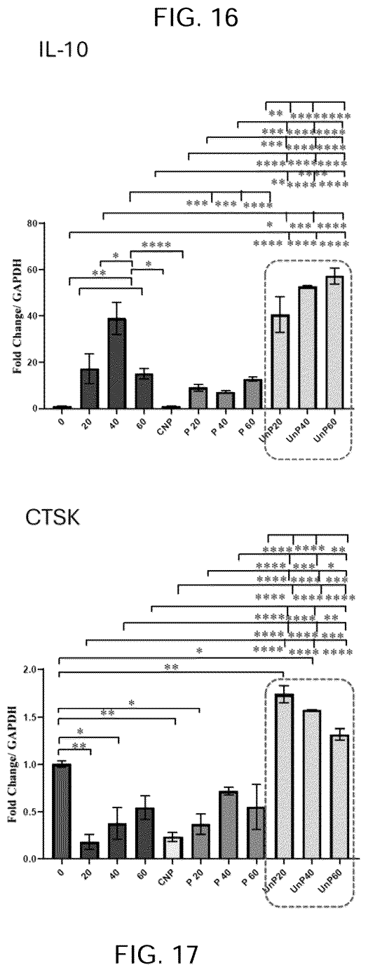

FIG. 16* shows the effect** of LaEPs-CNPs on amounts of the pro-healing factor interleukin-10 (IL-10). Under simulated harmful chronic inflammatory conditions IL-10 (pro-healing factor) is significantly elevated in the LaEPs/CNPs (with unbound LaEPs) groups.

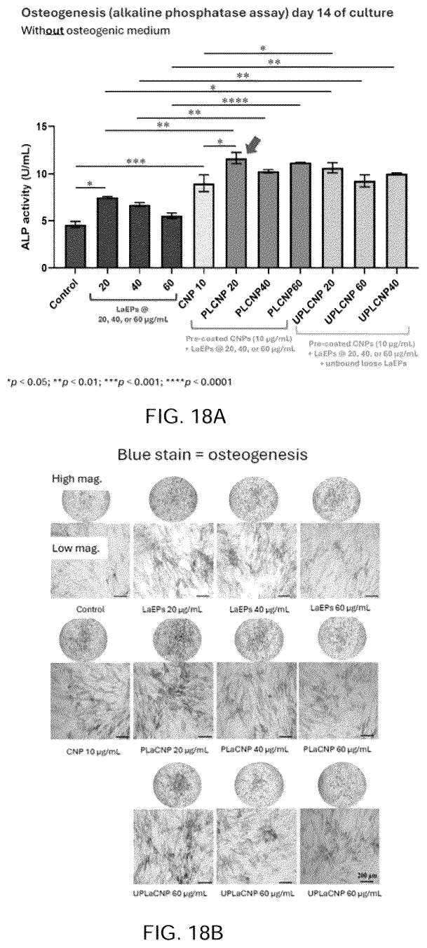

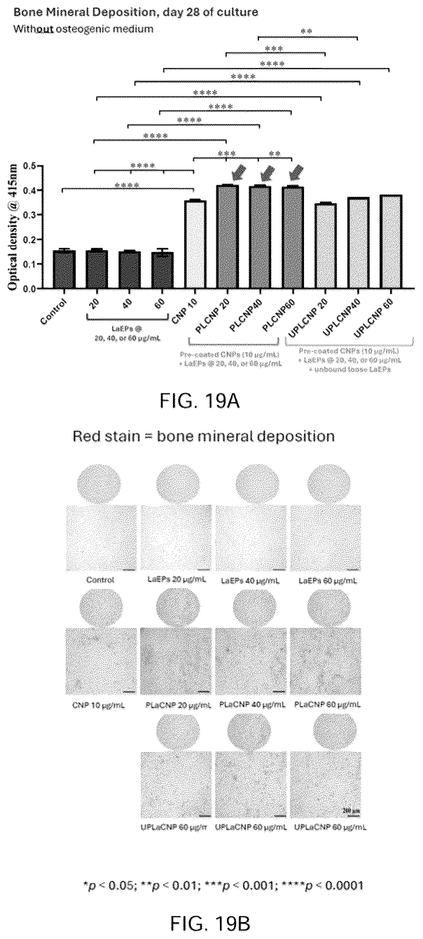

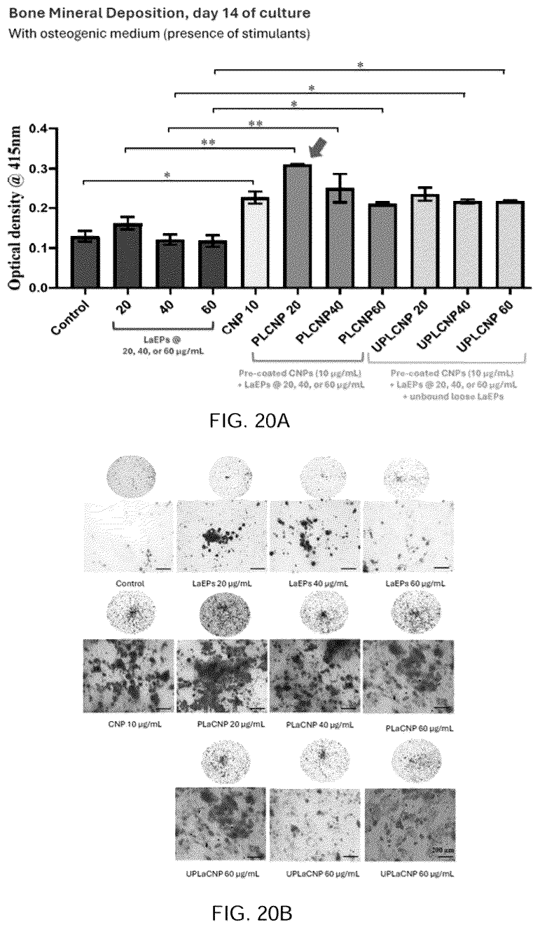

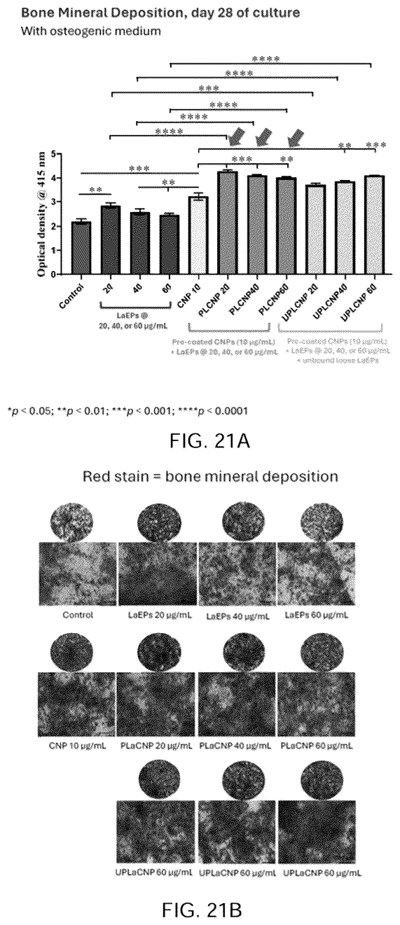

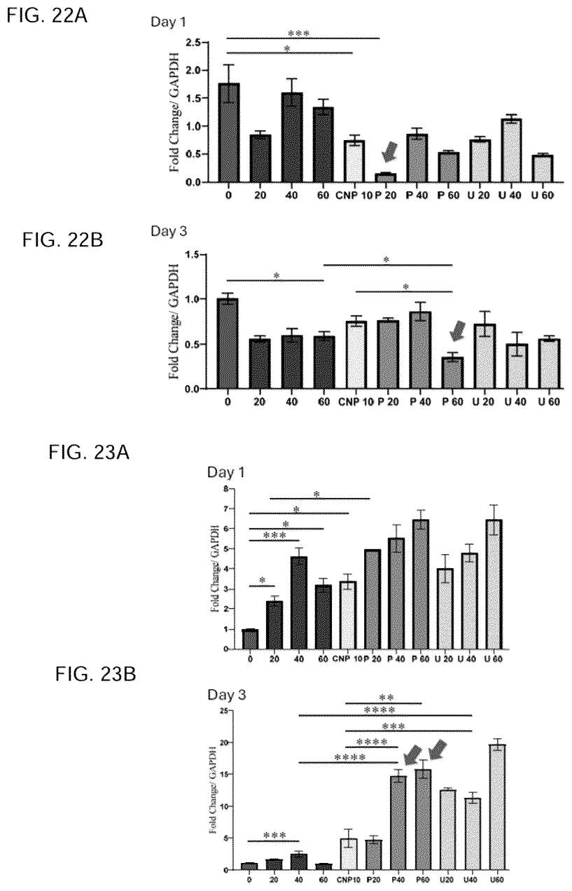

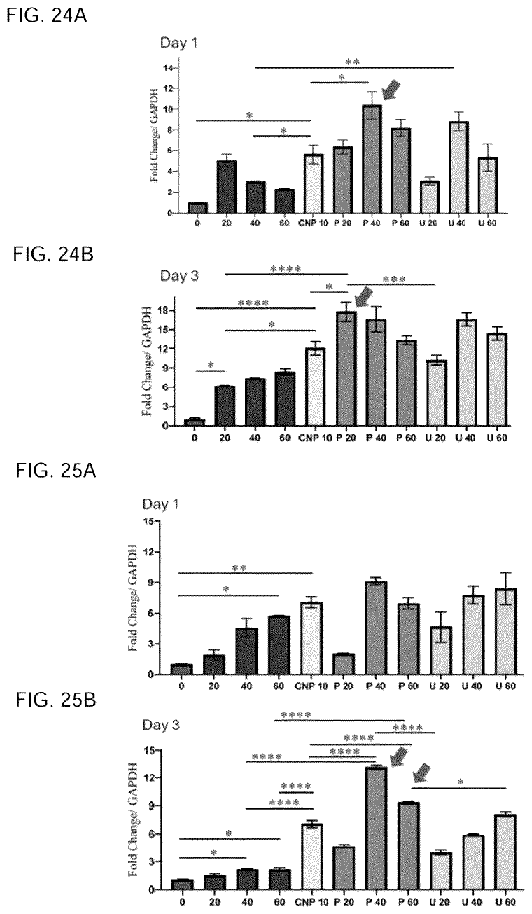

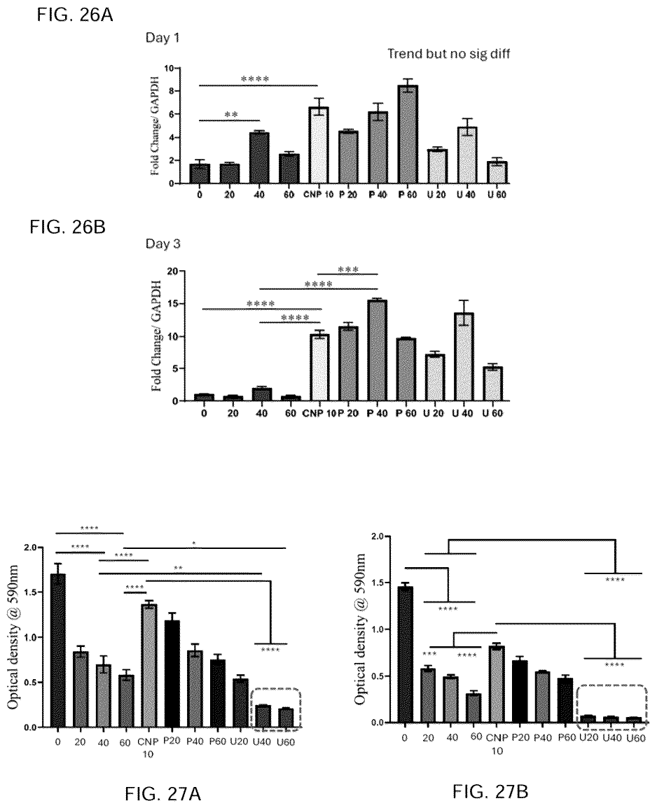

FIG. 17* shows the effect** of LaEPs-CNPs on amounts of the macrophage activator cathepsin K (CTSK). Under simulated harmful chronic inflammatory conditions, Cathepsin K (CTSK) is significantly elevated in the LaEPs/CNPs (with unbound LaEPs) groups. This is relevant, as the previous study of the instant inventors has shown that CTSK is a key factor that activates macrophages. When activated, the macrophages will engulf foreign bodies (e.g., harmful bacteria, debris, cancer cells) more. The response seen here indicates that coated CNPs in the presence of unbound LaEPs may significantly activate macrophages so they engulf and kill more bacteria and cancer cells than they would do normally.

Graphs* of FIGS. 12-17: first five columns left to right represent experimental controls, untreated control, LaEPs controls (20 μg/ml, 40 μg/ml, and 60 μg/ml), CNPs control (10 μg/ml) and next six columns left to right represent tested nanocompositions, coated CNPs (20 μg/ml LaEPs, 40 μg/ml LaEPs, and 60 μg/ml LaEPs) and coated CNPs+unbound LaEPs 20 μg/ml LaEPs, 40 μg/ml LaEPs, and 60 μg/ml LaEPs).

Graphs**of FIGS. 13-17: macrophages were pretreated with lipopolysaccharide (LPS) to simulate a chronic inflammatory condition akin to that seen in the human body.

### Experimental Example 3 (Part A-Part B)— LaEPs/CNPs: Cerium Oxide Nanoparticles (CNPs) Coated with Extracellular Proteins Isolated from Gut Probiotic Lactobacillus acidophilus (LaEPs) Boost New Bone Formation (Part A) and Modulate Both Anti-Inflammatory and Pro-Inflammatory Gene Expression (Part B)

### Introduction

Experiment 3 demonstrates that pre-coating cerium oxide nanoparticles (CNPs) with Lactobacillus acidophilus extracellular proteins (LaEPs) stimulates and boosts new bone formation to a statistically greater degree than either the CNPs alone or LaEPs alone. Further, the experiment shows that when cells are damaged by exposure to harmful levels of radiation, the LaEPs/CNPs (microbiome-ceria nanocompositions) statistically downregulate expression of pro-inflammatory (harmful) proteins while upregulating anti-inflammatory (pro-healing) proteins that helps to counteract the damage caused by the radiation. The LaEPs/CNPs were more effective at this gene regulation control than when either the CNPs or LaEPs were administered alone.

### General Methods

This experiment included examining the fate of human-derived mesenchymal stem cells, for example, measuring whether the stem cells were encouraged (or were not encouraged) to move toward forming osteoblasts (bone cells). The osteoblast cells are responsible for depositing new bone tissue in the body. An alkaline phosphatase dye was used to measure osteogenesis (whether the mesenchymal stem cells were turning into bone cells). The blue stain that results from the dye is an indication of the stem cells becoming osteoblasts.

### Results and Discussion

### Part A:

FIGS. 18A-B show data derived from an osteogenesis measurement experiment conducted on day 14 of culture without (in absence of) osteogenic medium. The LaEPs/CNPs (microbiome-ceria nanocompositions) induced the stem cells to differentiate into osteoblasts in the absence of any biological and/or chemical compounds that are usually given to stimulate the fate of stem cells toward osteoblast formation. Thus, the LaEPs/CNPs can be described as “osteoinductive” as they directly induce bone formation. FIG. 18A is a graph showing alkaline phosphatase activity on day 14 of culture. *p<0.05, **p<0.01, ***p<0.001, ****p<0.0001. These results show that the LaEPs alone (red bars, in concentrations of 20 μg/mL, 40 μg/mL, and 60 Lg/mL) significantly increased osteogenesis compared to the control group (green bar, untreated cells). The CNPs alone (yellow bar, in a concentration of 10 μg/ML) also significantly increased osteogenesis compared to the control group (green bar, untreated cells). The LaEPs/CNPs (orange bars, in concentrations of 20 μg/mL, 40 μg/mL, and 60 μg/mL) promote even more (statistically significant) osteogenesis and at a similar amount to the LaEPs/CNPs+any loose LaEPs in solution (aqua bars). For example, when either the LaEPs alone (red bar) are administered at a concentration of 20 μg/mL or the CNPs (yellow bar) are administered alone, they promote more new bone formation than the control (green bar, untreated cells). However, when the PLCNP 20 group (CNPs having LaEPs at a concentration of 20 μg/mL) is compared, there is a statistically significant increase in new bone formation compared to the CNPs alone or LaEPs alone. No differing trends are seen when the orange and aqua bars are compared. When the LaEPs are given at a concentration of 20 μg/mL, both the orange and the aqua groups increase formation of new bone to a similar amount. Overall, these results evidence a synergistic (or additive) and beneficial effect that happens when the CNPs are coated with LaEPs. FIG. 18B shows micrographs (in both high and low magnification) indicating osteogenesis (blue stain, alkaline phosphatase dye) in samples measured in FIG. 18A.

FIGS. 19A-B show data derived from a bone mineral deposition experiment conducted on day 28 of culture without (in absence of) osteogenic medium. This experiment measured whether the LaEPs/CNPs (orange bars) increased deposition of bone minerals when compared to either the CNPs alone (yellow bar) or the LaEPs alone (red bars). The alkaline phosphatase stain is a measure of the early events that happen in new bone formation, while bone mineral deposition gives an indication of how the later events of bone formation are influenced as the bone matrix is being deposited. The LaEPs alone (red bars) did not increase this later phase of bone formation, while the CNPs alone (yellow bar) did significantly increase mineral deposition. The LaEPs/CNPs (orange bars) further increased deposition of bone minerals beyond either the LaEPs or CNPs alone. This result was statistically significant for all three concentrations, 20 μg/mL, 40 μg/mL, and 60 μg/ML. The LaEPs/CNPs+loose LaEPs (aqua bars) did not perform as well the LaEPs/CNPs (orange bars, minus loose LaEPs). FIG. 19A is a graph showing optical density @415 nm on day 28 of culture. *p<0.05, **p<0.01, ***p<0.001, ****p<0.0001. FIG. 19B shows micrographs indicating bone mineral deposition (red stain) in samples measured in FIG. 19A. Red stain=bone mineral deposition. Overall, these results show that in the absence of any biological and/or chemical compounds given to stimulate the fate of stem cells toward osteoblast formation, the LaEPs/CNPs significantly increased the amount of bone mineral that formed, an indication that the LaEPs/CNPs are more potent in driving the creation of new bone (during later stages) compared to either the CNPs or LaEPs alone.

Whether the LaEPs/CNPs (orange bars) increased the deposition of bone minerals when in the presence of conventionally used chemicals, such as osteogenic medium, that drive stem cells toward new bone formation was also measured. FIGS. 20A-B show data derived from a bone mineral deposition experiment conducted on day 14 of culture with (in presence of) osteogenic medium. FIG. 20 is a graph showing optical density @415 nm on day 14 of culture with (in presence of) osteogenic medium. *p<0.05, **p<0.01, ***p<0.001, ****p<0.0001. At 20 μg/m L, the LaEPs/CNPs (orange bars) are more potent in stimulating the deposition of bone minerals than either the CNPs alone (yellow bars) or the LaEPs alone (red bars). The LaEPs/CNPs+loose LaEPs (aqua bars) did not perform as well the LaEPs/CNPs (orange bars, minus loose LaEPs). FIG. 20B shows micrographs indicating bone mineral deposition (red stain) in samples measured in FIG. 20A. Red stain=bone mineral deposition. Overall, these results show that in the presence of osteogenic medium given to stimulate the fate of stem cells toward osteoblast formation, the LaEPs/CNPs significantly increased the amount of bone mineral that formed, an indication that the LaEPs/CNPs are more potent in driving the creation of new bone (during later stages) compared to either the CNPs or LaEPs alone.

FIGS. 21A-B show data derived from a bone mineral deposition experiment conducted on day 28 of culture with (in presence of) osteogenic medium. FIG. 21A is a graph showing optical density @415 nm on day 28 of culture. *p<0.05, **p<0.01, ***p<0.001, ****p<0.0001. FIG. 21B shows micrographs indicating bone mineral deposition (red stain) in samples measured in FIG. 21A. Red stain=bone mineral deposition. As the cells have had more time (28 days vs. 14 days) to grow and deposit new material, the red stain and levels of deposition are more pronounced than measured in FIG. 20B. These results also show that the LaEPs/CNPs significantly increased the amount of bone mineral that formed, indicating that this combination was synergistically more potent in driving the formation of new bone mineral compared to either the CNPs or LaEPs alone. A synergistic effect is also evident in the aqua bar group and at the higher LaEPs concentrations of 40 μg/mL and 60 μg/mL.

### Part B:

Next, the ability of the LaEPs/CNPs to heal injured cells was tested. Macrophages are involved in processes that either promote or quench harmful levels of inflammation and thus, were selected as test cells for this part of the experiment. The macrophages were exposed to harmful levels (7 Gy) of ionizing radiation. Ionizing radiation damages macrophages causing them to release pro-inflammatory factors that in turn perpetuate an inflammatory response that damages other cells in the surrounding area. Alternatively, macrophages, in certain circumstances, can be pushed towards releasing anti-inflammatory factors that can promote a healing response. In this case, the macrophages were exposed to harmful radiation (7 Gy) to test whether the LaEPs/CNPs are adept at reducing the level of damaging pro-inflammatory factors, while also increasing the level of helpful anti-inflammatory factors.

The experimental groups used were:
0=Control Group (green bar)
20=20 μg/mL LaEPs (red bar)
40=40 μg/mL LaEPs (red bar)
60=60 μg/mL LaEPs (red bar)
CNP 10=CNPs @10 g/ML (yellow bar)
P20=CNPs coated with 20 μg/mL LaEPs (orange)
P40=CNPs coated with 40 μg/mL LaEPs (orange)
P60=CNPs coated with 60 μg/mL LaEPs (orange)
U20:=CNPs coated with 20 g/mL LaEPs+any unbound LaEPs present in solution (aqua)
U40=CNPs coated with 40 μg/mL LaEPs+any unbound LaEPs present in solution (aqua)
U60=CNPs coated with 60 μg/mL LaEPs+any unbound LaEPs present in solution (aqua)

FIGS. 22A-B show data derived from a RANKL (receptor activator of nuclear factor Kappa-B ligand) gene expression measurement experiment using quantitative PCR (qPCR). FIG. 22A is a graph showing gene expression of the pro-inflammatory factor RANKL on day 1 of culture. *p<0.05, **p<0.01, ***p<0.001, ****p<0.0001. After day 1 of culture, the LaEPs alone, the CNPs alone, and the LaEPs/CNPs all drove down harmful levels of RANKL. The LaEPs/CNPs, at the concentration of 20 μg/mL, show a clear synergistic trend with much lower levels of RANKL expression measured. However, no statistically significant difference was measured. FIG. 22B is a graph showing gene expression of the pro-inflammatory factor RANKL on day 3 of culture. *p<0.05, **p<0.01, ***p<0.001, ****p<0.0001. After 3 days of culture, the LaEPs given at 60 μg/mL (red bar), CNPs alone (yellow bar), and LaEPs/CNPs P60 (orange bar) all significantly decreased harmful levels of RANKL. Notably, the P60 synergistically promotes significantly more dampening of RANKL expression than either the CNPs or LaEPs (60 μg/mL) alone.

FIGS. 23A-B show data derived from a TGFβ (transforming growth factor beta) gene expression measurement experiment using quantitative PCR (qPCR). FIG. 23A is a graph showing gene expression of the anti-inflammatory factor TGFβ on day 1 of culture. *p<0.05, **p<0.01, ***p<0.001, ****p<0.0001. After day 1 of culture, a trend is seen where the LaEPs alone, the CNPs alone, and the LaEPs/CNPs all increase beneficial levels of TGFβ expression. A clear trend is seen in which the LaEPs/CNPs (orange bars) synergistically drive higher levels of TGFβ expression compared to either CNPs alone (yellow) or the LaEPs alone (red), but the data is not statistically significant for confirmation of this pattern. FIG. 23B is a graph showing gene expression of the anti-inflammatory factor TGFβ on day 3 of culture. *p<0.05, **p<0.01, ***p<0.001, ****p<0.0001. After 3 days of culture, the LaEPs at concentration of 40 μg/mL (red bar) and the CNPs alone each induced significantly higher TGFβ expression compared to control cells (green bar, untreated cells). When CNPs were coated with 40 or 60 μg/mL of LaEPs a further significant effect in increasing expression is seen confirming the synergistic effect that coating the CNPs (with the LaEPs) has on stimulating a pro-healing response.

FIGS. 24A-B show data derived from an IL-10 (interleukin-10) gene expression measurement experiment using quantitative PCR (qPCR). FIG. 24A is a graph showing gene expression of the anti-inflammatory factor IL-10 on day 1 of culture. *p<0.05, **p<0.01, ***p<0.001, ****p<0.0001. After day 1 of culture, a trend is seen where the CNPs alone and the LaEPs/CNPs (40 μg/mL) both significantly increase beneficial levels of IL-10 expression. The LaEPs/CNPs (40 μg/mL) increase pro-healing IL-10 expression confirming the synergistic effect that the coating technique has on inducing a beneficial cellular response. FIG. 24B is a graph showing gene expression of the anti-inflammatory factor IL-10 on day 3 of culture. *p<0.05, **p<0.01, ***p<0.001, ****p<0.0001. After 3 days of culture, the LaEPs/CNPs (20 μg/mL) confirm a beneficial synergistic effect compared to both the CNPs alone and the LaEPs (20 μg/mL) alone.

FIGS. 25A-B show data derived from a CD-163 (cluster of differentiation-163) gene expression measurement experiment using quantitative PCR (qPCR). FIG. 25A is a graph showing gene expression of the anti-inflammatory factor CD-163 on day 1 of culture. *p<0.05, **p<0.01, ***p<0.001, ****p<0.0001. After day 1 of culture, the LaEPs given at a concentration of 60 μg/mL significantly increase beneficial CD-163 expression. A synergistic trend is measured when the CNPs were coated with LaEPs at a concentration of 40 μg/mL, however no significant difference was found. FIG. 25B is a graph showing gene expression of the anti-inflammatory factor CD-163 on day 3 of culture. *p<0.05, **p<0.01, ***p<0.001, ****p<0.0001. After 3 days of culture, the LaEPs at a concentration of 40 μg/mL and the CNPs (alone) both significantly increase levels of beneficial CD-163 expression. However, a further and substantial increase is measured when the LaEPs (40 and 60 μg/mL) are coated onto CNPs confirming a synergistic pro-healing response. When the LaEPs/CNPs include loose LaEPs in the solution (aqua bars), this synergistic and beneficial response is not seen.

FIGS. 26A-B show data derived from a CD-206 (cluster of differentiation-206) gene expression measurement experiment using quantitative PCR (qPCR). FIG. 26A is a graph showing gene expression of the anti-inflammatory factor CD-206 on day 1 of culture. *p<0.05, **p<0.01, ***p<0.001, ****p<0.0001. After day 1 of culture, the LaEPs at a concentration of 40 pig/mL as well as the CNPs alone significantly increase beneficial CD-206 expression. A synergistic trend is observed when CNPs were coated with 60 pig/mL of LaEPs. However, no statistically significant difference was found. FIG. 26B is a graph showing gene expression of the anti-inflammatory factor CD-206 on day 3 of culture. *p<0.05, **p<0.01, ***p<0.001, ****p<0.0001. After 3 days of culture, the CNP alone significantly increases levels of beneficial CD-206 expression compared to the control. However, a further and substantial increase is measured when the LaEPs, in a concentration of 40 μg/mL, are coated onto the CNPs confirming a synergistic pro-healing response.

Overall, these gene expression measurements show that the LaEPs/CNPs significantly decreased the level of pro-inflammatory factors and increased the level of anti-inflammatory factors, indicating that the LaEPs/CNPs are synergistically more potent in driving down inflammation and promoting more of a healing response compared to either the CNPs alone or the LaEPs alone.

### Additional Experimental Data

Previous work has shown that CNPs do not show sufficient efficacy against pathogenic Gram-negative bacteria Pseudomonas aeruginosa or Gram-positive Staphylococcus aureus bacteria forming biofilm. However, experiments similar to those discussed above (bacterial count: 10⁶ CFU/ml; temperature: 37 °C; incubation time: 24 hours; and medium: Luria-Bertani) show that the LaEPs at concentrations of 20, 40 and 60 μg/mL do prevent biofilm formation in a dose-dependent manner, and that this effect is further augmented when the pre-coated CNPs are in the presence of the loose/unbound LaEPs. A statistically significant effect was measured against both P. aeruginosa (FIG. 27A) and S. aureus (FIG. 27B).

For P. aeruginosa, the CNPs alone (at a dose of 10 μg/mL: CNP 10) slightly reduce biofilm formation (p<0.05). However, the pre-coated CNPs at concentrations of 40 and 60 μg/mL in the presence of unbound/loose LaEPs (U40 and U60), synergistically reduce/prevent P. aeruginosa biofilm formation. Statistically, the U40 and U60 groups significantly reduce biofilm formation more than either the CNPs alone or the respective LaEPs (alone) groups, demonstrating an augmented and synergistic effect.

For S. aureus, the CNPs alone (at a dose of 10 μg/mL: CNP 10) reduce biofilm formation (p<0.0001). However, the pre-coated CNPs at concentrations of 20, 40 and 60 μg/mL in the presence of unbound/loose LaEPs (U20, U40 & U60), synergistically prevents S. aureus biofilm formation. Statistically, the U20, U40, and U60 groups significantly reduce biofilm formation more than either the CNPs alone or the respective LaEPs (alone) groups, demonstrating an augmented and synergistic effect in preventing S. aureus biofilm formation.

### Conclusion of Specification

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended representative claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the representative claims.

After reading the disclosure, skilled artisans will appreciate that certain features are, for clarity, described herein in the context of separate embodiments can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following representative claims be interpreted to embrace all such variations and modifications. Thus, the nanocompositions, compositions, nanoparticles, pharmaceutical compositions, kits, implants, and/or methods described herein are presently representative of the preferred embodiments, are intended to be exemplary and are not intended as limitations on the scope.

### REFERENCES

[1]K. Keely Boyle, S. Rachala, S. R. Nodzo, Curr. Rev. Musculoskelet. Med. 2018, 11, 357.
[2]B. D. Springer, S. Cahue, C. D. Etkin, D. G. Lewallen, B. J. Mcgrory, Arthroplast. Today 2017, 3, 137.
[3]R. E. Delanois, J. B. Mistry, C. U. Gwam, N. S. Mohamed, U. S. Choksi, M. A. Mont, J. Arthroplasty 2017, 32, 2663.
[4]A. Eka, A. F. Chen, Ann. Transl. Med. 2015, 3, 233.
[5]S. Kurtz, K. Ong, E. Lau, F. Mowat, M. Halpern, J. Bone Jt. Surg. 2007, 89, 780.
[6]W. Zimmerli, A. Trampuz, P. E. Ochsner, N. Engl. J Med. 2004, 351,1645.
[7]K. L. Ong, S. M. Kurtz, E. Lau, K. J. Bozic, D. J. Berry, J. Parvizi, J. Arthroplasty 2009, 24, 105.
[8]P. Izakovicova, O. Borens, A. Trampuz, EFORT Open Rev 2019, 4, 482.
[9]B. H. Kapadia, R. A. Berg, J. A. Daley, J. Fritz, A. Bhave, M. A. Mont, Lancet 2016, 387, 386.
[10]A. H. Elmaidomy, N. H. Shady, K. M. Abdeljawad, M. B. Elzamkan, H. H. Helmy, E. A. Tarshan, A. N. Adly, Y. H. Hussien, N. G. Sayed, A. Zayed, U. R. Abdelmohsen, RSC Adv. 2022, 12, 29078.
[11]M. Taha, H. Abdelbary, F. P Ross, A. V. Carli, Curr. Rev. Musculoskelet. Med. 2018, 11, 380.
[12]J. S. Day, E. Lau, K. L. Ong, G. R. Williams, M. L. Ramsey, S. M. Kurtz, J. Shoulder Elb. Surg. 2010, 19, 1115.
[13]M. Sloan, A. Premkumar, N. P. Sheth, J. Bone Jt. Surg. 2018, 100, 1455.
[14]Y. M. Wi, R. Patel, Infect. Dis. Clin. North Am. 2018, 32, 915.
[15]L. Hall-Stoodley, J. W Costerton, P. Stoodley, Nat. Rev. Microbiol. 2004, 2, 95.
[16]W. Yin, Y. Wang, L. Liu, J. He, Int. J. Mol. Sci. 2019, 20, 3423.
[17]H. Koo, R. N. Allan, R. P. Howlin, P. Stoodley, L. Hall-Stoodley, Nat. Rev. Microbiol. 2017, 15, 740.
[18]U. Romling, S. Kjelleberg, S. Normark, L. Nyman, B. E. Uhlin, B. Akerlund, J. Intern. Med. 2014, 276, 98.
[19]C. V Eiff, C. Heilmann, G. Peters, Eur. J. Clin. Microbiol. Infect. Dis. 1999, 18, 843.
[20]A. Rakow, C. Perka, A. Trampuz, N. Renz, Clin. Microbiol. Infect. 2019, 25, 845.
[21]D. R. Murdoch, S. A. Roberts, V. G. Fowler, M. A. Shah, S. L. Taylor, A. J. Morris, G. R. Corey, Clin. Infect. Dis. 2001, 32, 647.
[22]G. J. Moran, A. Krishnadasan, R. J. Gorwitz, G. E. Fosheim, L. K. Mcdougal, R. B. Carey, D. A. Talan, N. Engl. J Med. 2006, 355, 666.
[23] S. J. Volin, S. H. Hinrichs, K. L. Garvin, Clin. Orthop. Relat. Res. 2004, 427, 94.
[24] D. J. Kilgus, D. J. Howe, A. Strang, Clin. Orthop. Relat. Res. 2002, 404, 116.
[25] L. Pulido, E. Ghanem, A. Joshi, J. J. Purtill, J. Parvizi, Clin. Orthop. Relat. Res. 2008, 466, 1710.
[26] Y. Mittal, T. K. Fehring, S. M. Odum, A. Hanssen, C. Marculescu, D. Osmon, J. Bone Jt. Surg. 2007, 89, 1227.
[27] W. Zimmerli, P. Sendi, APMIS 2017, 125, 353.
[28] J. Lienau, K. Schmidt-Bleek, A. Peters, F. Haschke, G. N. Duda, C. Perka, H. J. Bail, N. Schutze, F. Jakob, H. Schell, J. Orthop. Res. 2009, 27, 1133.
[29] A. Kienzle, S. Walter, P. Von Roth, M. Fuchs, T. Winkler, M. Muller, JBJS Open Access 2020, 5, e2000026.
[30] T. N. Crotti, A. A. S. S. K. Dharmapatni, E. Alias, D. R. Haynes, J. Immunol. Res. 2015, 2015, 281287.
[31] L. Biedermann, E. Bandick, Y. Ren, S. Tsitsilonis, S. Donner, M. Muller, G. Duda, C. Perka, A. Kienzle, JBJS Open Access 2023, 8, e22.
[32] R. K. Srivastava, H. Y. Dar, P. K. Mishra, Front. Immunol. 2018, 9, 657.
[33]Y. Saxena, S. Routh, A. Mukhopadhaya, Front. Immunol. 2021, 12, 687037.
[34] M. McClung, Arthritis Res. Ther. 2007, 9, S3.
[35] B. F. Boyce, L. Xing, Arch. Biochem. Biophys. 2008, 473, 139.
[36] S. M. Horowitz, M. A. Purdon, Calcif Tissue Int. 1995, 57, 301.
[37] U. Dapunt, T. Giese, S. Stegmaier, A. Moghaddam, G. M. Hansch, BMC Musculoskelet. Disord. 2016, 17, 243.
[38] D, T. Graves, Front. Biosci. 1999, 4, d571.
[39] Y. Tanaka, S. Nakayamada, Y. Okada, Curr. Drug Target-Inflammation Allergy 2005, 4, 325.
[40] N. Aaron, S. Costa, C. J. Rosen, L. Qiang, Front. Endocrinol. 2022, 13, 853765.
[41] F. Wei, C. J. Neal, T. S. Sakthivel, Y. Fu, M. Omer, A. Adhikary, S. Ward, K. M. Ta, X. Moxon, M. Molinari, J. Asiatico, M. Kinzel, S. N. Yarmolenko, V. S. Cheong, N. Orlovskaya, R. Ghosh, S. Seal, M. Coathup, Bioact. Mater. 2023, 21, 547.
[42] Y. Fan, J.-I. Hanai, P. T. Le, R. Bi, D. Maridas, V. Demambro, C. A. Figueroa, S. Kir, X. Zhou, M. Mannstadt, R. Baron, R. T. Bronson, M. C. Horowitz, J. Y. Wu, J. P. Bilezikian, D. W. Dempster, C. J. Rosen, B. Lanske, Cell Metab. 2017, 25, 661.
[43]A. G. Veldhuis-Vlug, C. J. Rosen, J. Intern. Med. 2018, 283, 121.
[44] T. Y. Kim, A. L. Schafer, Curr. Osteoporos. Rep. 2016, 14, 337.
[45] F. Wei, Z. K. Tuong, M. Omer, C. Ngo, J. Asiatico, M. Kinzel, A. S. Pugazhendhi, A. R. Khaled, R. Ghosh, M. Coathup, Bone Res. 2023, 11, 34.
[46] L. Hu, C. Yin, F. Zhao, A. Ali, J. Ma, A. Qian, Int. J. Mol. Sci. 2018, 19, 360.
[47]K. Redlich, J. S. Smolen, Nat. Rev. Drug Discovery 2012, 11, 234.
[48] T. Jiang, H. Gu, J. Wei, Front. Pharmacol. 2022, 13, 930053.
[49] S. Fukuda, H. Ohno, Semin. Immunopathol. 2014, 36, 103.
[50] H. Yousf, G. B. Tomar, R. K. Srivastava, Int. J. Immunother. Cancer Res. 2015, 1, 18.
[51] F. L. Collins, N. D. Rios-Arce, J. D. Schepper, N. Parameswaran, L. R. Mccabe, Microbiol. Spectr. 2017, 5, 4.
[52] S. Satpute, N. Mone, P. Das, A. Banpurkar, I. Banat, Appl. Sci. 2018, 8, 1555.
[53]C. Vuotto, F. Longo, G. Donelli, Int. J. Oral Sci. 2014, 6, 189.
[54] T. D. Lawley, A. W. Walker, Immunology 2013, 138, 1.
[55] H. Sikorska, W. Smoragiewicz, Int. J. Antimicrob. Agents 2013, 42, 475.
[56] A. Jeyanathan, A. Ramalhete, G. Blunn, H. Hibbs, C. A. Pumilia, T. Mechmongkol, J. Lovejoy, M. J. Coathup, J. Biomed. Mater. Res., Part B 2021, 109, 1634.
[57]Y. Alexandre, R. Le Berre, G. Barbier, G. Le Blay, BMC Microbiol. 2014, 14, 107.
[58] N. Ibnou-Zekri, S. Blum, E. J. Schiffrin, T. Von Der Weid, Infect. Immun. 2003, 71, 428.
[59] B. Zhou, D. Zhang, Exp. Ther. Med. 2018, 15, 2985.
[60] T. S. Trunk, H. Khalil, C. Leo, AIMS Microbiol. 2018, 4, 140.
[61] Y. Tuo, H. Yu, L. Ai, Z. Wu, B. Guo, W. Chen, J. Dairy Sci. 2013, 96, 4252.
[62]J. A. Younes, H. C. Van Der Mei, E. Van Den Heuvel, H. J. Busscher, G. Reid, PLoS One 2012, 7, e36917.
[63] X. Chen, F. Tian, X. Liu, J. Zhao, H.-P. Zhang, H. Zhang, W. Chen, J. Dairy Sci. 2010, 93, 5627.
[64] M. Nikolic, B. Jovcic, M. Kojic, L. Topisirovic, Eur. Food Res. Technol. 2010, 231, 925.
[65] C. L. Ferreira, L. Grzeskowiak, M. C. Collado, S. Salminen, J. Food Prot. 2011, 74, 1482.
[66]M. C Collado, J. Meriluoto, S. Salminen, J Microbiol. Methods 2007, 71, 71.
[67] A. Zawistowska-Rojek, A. Kosmider, K. Stepien, S. Tyski, Arch. Microbiol. 2022, 204, 285.
[68] S. Halder, K. K. Yadav, R. Sarkar, S. Mukherjee, P. Saha, S. Haldar, S. Karmakar, T. Sen, SpringerPlus 2015, 4, 672.
[69] R. Somayaji, T. Lynch, J. N. Powell, D. Gregson, BMC Infect. Dis. 2016, 16, 634.
[70]C. Tan, J. L. Howard, L. Bondy, Can. Med. Assoc. J. 2020, 192, E1357.
[71] F. Ait Ouali, I. Al Kassaa, B. Cudennec, M. Abdallah, F. Bendali, D. Sadoun, N.-E. Chihib, D. Drider, Int. J. Food Microbiol. 2014, 191, 116.
[72] M. Koohestani, M. Moradi, H. Tajik, A. Badali, Vet. Res. forum an Int. Q. J. 2018, 9, 301.
[73] A. Darbandi, A. Asadi, M. Mahdizade Ari, E. Ohadi, M. Talebi, M. Halaj Zadeh, A. Darb Emamie, R. Ghanavati, M. Kakanj, J. Clin. Lab. Anal. 2022, 36, e24093.
[74] R. S. Flannagan, G. Cosio, S. Grinstein, Nat. Rev. Microbiol. 2009, 7, 355.
[75] S. Nagata, R. Hanayama, K. Kawane, Cell 2010, 140, 619.
[76] F. Wei, C. J. Neal, T. S. Sakthivel, T. Kean, S. Seal, M. J. Coathup, Mater. Sci. Eng. C. Mater. Biol. Appl. 2021, 124, 112041.
[77]R. N. Freiberger, C. A. M. Lopez, F. A. Sviercz, C. Cevallos, A. D. Guano, P. Jarmoluk, J. Quarleri, M. V. Delpino, Int. J. Mol. Sci. 2023, 24, 5617.
[78] K. R. Georgiou, S. K. Hui, C. J. Xian, Am. J Stem Cells 2012, 1, 205.
[79] Q. Chen, P. Shou, C. Zheng, M. Jiang, G. Cao, Q. Yang, J. Cao, N. Xie, T. Velletri, X. Zhang, C. Xu, L. Zhang, H. Yang, J. Hou, Y. Wang, Y. Shi, Cell Death Differ. 2016, 23, 1128.
[80] A. T. J. C., Eur. Spine J. 2001, 10, S96.
[81] J. Haaber, M. T. Cohn, D. Frees, T. J. Andersen, H. Ingmer, PLoS One 2012, 7, e41075.
[82]D. Bujnakova, V. Kmet, J. Vet. Med. Ser. B 2002, 49, 152.
[83]G. Reid, J. A. Mcgroarty, R. Angotti, R. L. Cook, Can. J. Microbiol. 1988, 34, 344.
[84] B. Giordani, C. Parolin, B. Vitali, Front. Med. Technol. 2021, 3, 769172.
[85] F. C. Mgomi, Y.-R Yang, G. Cheng, Z.-Q. Yang, Biofilm 2023, 5, 100118.
[86] M. P. Taranto, M. L. Fernandez Murga, G. Lorca, G. F. Valdez, J. Appl. Microbiol. 2003, 95, 86.
[87] R. Sengupta, E. Altermann, R. C. Anderson, W. C. Mcnabb, P. J. Moughan, N. C. Roy, Mediators Inflamm. 2013, 2013, 237921.
[88] B. Alizadeh Behbahani, M. Noshad, F. Falah, Microb. Pathog. 2019, 136, 103677.
[89] M. Mohamadzadeh, E. A. Pfeiler, J. B. Brown, M. Zadeh, M. Gramarossa, E. Managlia, P. Bere, B. Sarraj, M. W. Khan, K. C. Pakanati, M. J Ansari, S. O'flaherty, T. Barrett, T. R. Klaenhammer, Proc. Natl. Acad. Sci. U.S.A. 2011, 108, 4623.
[90] E. K. Call, Y. J. Goh, K. Selle, T. R. Klaenhammer, S. O'flaherty, Microbiology 2015, 161, 311.
[91] Y. J. Goh, T. R. Klaenhammer, Appl. Environ. Microbiol. 2010, 76, 5005.
[92] B. R. Johnson, J. Hymes, R. Sanozky-Dawes, E. D. Henriksen, R. Barrangou, T. R. Klaenhammer, Appl. Environ. Microbiol. 2016, 82, 134.
[93] S. J. O'flaherty, T. R. Klaenhammer, Microbiology 2010, 156, 3360.
[94] C. M. C. Chapman, G. R. Gibson, I. Rowland, Anaerobe 2014, 27, 71.
[95] B. Sadowska, E. Walencka, M. Wieckowska-Szakiel, B. Ro'zalska, Folia Microbiol. 2010, 55, 497.
[96] J. Bhola, R. Bhadekar, BMC Complement. Altern. Med. 2019, 19, 70.
[97]N. Saidi, H. Saderi, P. Owlia, M. Soleimani, Adv. Biomed. Res. 2023, 12, 50.
[98] M. A. Diaz, M. R. Alberto, E. G. Vega-Hissi, S. N. Gonzalez, M. E. Arena, Arab. J. Sci. Eng. 2022, 47, 241.
[99] D. Abedi, S. Feizizadeh, V. Akbari, A. Jafarian-Dehkordi, Res. Pharm. Sci. 2013, 8, 260.
[100]J. S. Ong, T. D. Taylor, C. C. Yong, B. Y. Khoo, S. Sasidharan, S. B. Choi, H. Ohno, M. T. Liong, Probiotics Antimicrob. Proteins 2020, 12, 125.
[101]S. Jung, O.-J Park, A. R Kim, K. B. Ahm, D. Lee, K.-Y. Kum, C.-H. Yun, S. H. Han, J. Microbiol. 2019, 57, 310.
[102]N.-N. Kim, W. J. Kim, S.-S. Kang, Food Control 2019, 98, 274.
[103]L. Cisneros, N. Cattelan, M. I. Villalba, C. Rodriguez, D. O. Serra, O. Yantorno, S. Fadda, Lett. Appl. Microbiol. 2021, 73, 247.
[104]Y. Oishi, I. Manabe, Int. Immunol. 2018, 30, 511.
[105]W. Qiao, H. Xie, J. Fang, J. Shen, W. Li, D. Shen, J. Wu, S. Wu, X. Liu, Y. Zheng, K. M. C. Cheung, K. W. K. Yeung, Biomaterials 2021, 276, 121038.
[106]L. Szulc-Dabrowska, M. Bossowska-Nowicka, J. Struzik, F. N. Toka, Front. Cell. Infect. Microbiol. 2020, 10, 601072
[107]F. Y. Mcwhorter, T. Wang, P. Nguyen, T. Chung, W. F. Liu, Proc. Natl. Acad. Sci. U.S.A. 2013, 110, 17253.
[108]J. L Stoger, M. J. J. Gijbels, S. Van Der Velden, M. Manca, C. M. Van Der Loos, E. A. L. Biessen, M. J. A. P. Daemen, E. Lutgens, M. P. J. De Winther, Atherosclerosis 2012, 225, 461.
[109]N. Fujiwara, K. Kobayashi, Curr. Drug Target-Inflammation Allergy 2005, 4, 281.
[110]N. Bonnet, E. Douni, G. Perreard Lopreno, M. Besse, E. Biver, S. Ferrari, J Bone Miner. Res. 2021, 36, 1636.
[111]K. Soe, D. M. H. Merrild, J.-M. Delaisse, Bone 2013, 56, 191.
[112]L. Mira-Pascual, A. N. Tran, G. Andersson, T. Nareoja, P. Lang, Int. J. Mol. Sci. 2020, 21, 538.
[113]R. Dai, Z. Wu, H. Y. Chu, J. Lu, A. Lyu, J. Liu, G. Zhang, Front. Cell Dev. Biol. 2020, 8, 433.
[114]Y. Cheng, H. Liu, J. Li, Y. Ma, C. Song, Y. Wang, P. Li, Y. Chen, Z. Zhang, PLoS One 2022, 17, e0277871.
[115]T. T. Hlaing, J. E. Compston, Ann. Clin. Biochem. Int. J. Lab. Med. 2014, 51, 189.
[116]R. Swaminathan, Clin. Chim. Acta 2001, 313, 95.
[117]S. Conus, H. Simon, Swiss Med. Wkly. 2010, 140, w13042.
[118]I. Fabrik, O. Bilkei-Gorzo, M. Oberg, D. Fabrikova, J. Fuchs, C. Sihlbom, M. Goransson, A. Hartlova, Life Sci. Alliance 2023, 6, e202201535.
[119]Y. S. Nanjundaiah, D. A. Wright, A. R. Baydoun, Z. Khaled, Z. Ali, P. Dean, M. H. Sarker, Front. Cell Dev. Biol. 2020, 8, 723.
[120]L. M. Rocha-Ramirez, R. A. Perez-Solano, S. L. Castanon-Alonso, S. S. Moreno Guerrero, A. Ramirez Pacheco, M. Garcia Garibay, C. Eslava, J. Immunol. Res. 2017, 2017, 4607491.
[121]C. Hidalgo-Cantabrana, M. A. Moro-Garcia, A. Blanco-Miguez, F. Fdez-Riverola, M. Olivan, L. J. Royo, S. Riestra, A. Margolles, A. Lourenco, R. Alonso-Arias, B. Sanchez, J. Funct. Foods 2020, 64, 103660.
[122]S. G. Bungau, T. Behl, A. Singh, A. Sehgal, S. Singh, S. Chigurupati, S. Vijayabalan, S. Das, V. R. Palanimuthu, Nutrients 2021, 13, 3376.
[123]O. M. S. Khedr, S. M. El-Sonbaty, F. S. M. Moawed, E. I. Kandil, B. E. Abdel-Maksoud, Nutr. Cancer 2022, 74, 1037.
[124]J. Zhou, J. Cheng, L. Liu, J. Luo, X. Peng, Foods 2023, 12, 275.
[125]H. Malmir, H.-S. Hjtahed, A.-R. Soroush, A. M. Mortazavian, N. Fahimfar, A. Ostovar, A. Esmaillzadeh, B. Larijani, S. Hasani-Ranjbar, Altern. Med. 2021, 2021, 3582989.
[126]L. A. T. Cannarella, N. L. Mari, C. C. Alcantara, T. M. V. Iryioda, N. T. Costa, S. R. Oliveira, M. A. B. Lozovoy, E. M. V. Reiche, I. Dichi, A. N. C. Simao, Nutrition 2021, 89, 111282.
[127]S.-C. Li, W.-F. Hsu, J-S. Chang, C.-K. Shih, Nutrients 2019, 11, 969.
[128]N. M. Breyner, C. Michon, C. S. De Sousa, P. B. Vilas Boas, F. Chain, V. A. Azevedo, P. Langella, J. M. Chatel, Front. Microbiol. 2017, 8, 114.
[129]C-Y. Chang, M. Tucci, R. C. Baker, Alcohol 2000, 20, 37.
[130]C.-Y. Lin, S.-H. Kao, L.-C. Hung, H.-J. Chien, W.-H. Wang, Y.-W Chang, Y.-H. Chen, Life 2021, 11, 411.
[131]S. Y. Ji, H.-J. Cha, I. M. N. Molagoda, M. Y. Kim, S. Y Kim, H. Hwangbo, H. Lee, G.-Y Kim, D.-H Kim, J. W. Hyun, H.-S. Kim, S. Kim, C.-Y. Jin, Y. H. Choi, Biomol. Ther. 2021, 29, 685.
[132]M. Nakayama, A. Nakamura, T. Hondou, H. Miyata, Int. J. Radiat. Biol. 2016, 92, 583.
[133]M. Bauer, M. Goldstein, M. Christmann, H. Becker, D. Heylmann, B. Kaina, Proc. Natl. Acad. Sci. U.S.A. 2011, 108, 21105.
[134]S. Pereira-Lopes, J. Tur, J. A. Calatayud-Subias, J. Lloberas, T. H. Stracker, A. Celada, Blood 2015, 126, 2502.
[135]T. Vico, C. Youssif, F. Zare, M. Comalada, C. Sebastian, J. Lloberas, A. Celada, Cells 2022, 11, 935.
[136]J. A. Hamilton, P. P. Tak, Arthritis Rheum. 2009, 60, 1210.
[137]S. Arora, K. Dev, B. Agarwal, P. Das, M. A. Syed, Immunobiology 2018, 223, 383.
[138]C. Schlundt, T. El Khassawna, A. Serra, A. Dienelt, S. Wendler, H. Schell, N. Van Rooijen, A. Radbruch, R. Lucius, S. Hartmann, G. N. Duda, K. Schmidt-Bleek, Bone 2018, 106, 78.
[139]F. Takahashi, K. Takahashi, K. Shimizu, R. Cui, N. Tada, H. Takahashi, S. Soma, M. Yoshioka, Y. Fukuchi, Lung 2004, 182, 173.
[140]M. Kreutz, R. Andreesen, S. Krause, A. Szabo, E. Ritz, H. Reichel, Blood 1993, 82, 1300.
[141]K. Chen, Y. Jiao, L. Liu, M. Huang, C. He, W. He, J. Hou, M. Yang, X. Luo, C. Li, Front. Cell Dev. Biol. 2020, 8, 598263.
[142]H. Y. Dar, P. Shukla, P. K. Mishra, R. Anupam, R. K. Mondal, G. B. Tomar, V. Sharma, R. K. Srivastava, Bone Reports 2018, 8, 46.
[143]N. Montazeri-Najafabady, Y. Ghasemi, M. H. Dabbaghmanesh, Y. Ashoori, P. Talezadeh, F. Koohpeyma, S. N. Abootalebi, A. Gholami, BMC Complement. Med. Ther. 2021, 21, 155.
[144]N. Montazeri-Najafabady, Y. Ghasemi, M. H. Dabbaghmanesh, P. Talezadeh, F. Koohpeyma, A. Gholami, Probiotics Antimicrob. Proteins 2019, 11, 1145.
[145]J. Zhai, S. Sun, J. Cheng, J. Wang, G. Jin, X. Xu, X. Liu, J. Zhao, C. Chen, W. Zhong, B. Wang, Future Microbiol. 2023, 18, 581.
[146]J. Masrour Roudsari, S. Mahjoub, Casp. J. Intern. Med. 2012, 3, 478.
[147]H. Harris, Clin. Chim. Acta 1990, 186, 133.
[148] S. Vimalraj, Gene 2020, 754, 144855.
[149]T. Komori, H. Yagi, S. Nomura, A. Yamaguchi, K. Sasaki, K. Deguchi, Y. Shimizu, R. T Bronson, Y.-H Gao, M. Inada, M. Sato, R. Okamoto, Y. Kitamura, S. Yoshiki, T. Kishimoto, Cell 1997, 89, 755.
[150]W. Huang, Front. Biosci. 2007, 12, 3068.
[151]M. J. Jeon, J. A. Kim, S. H. Kwon, S. W. Kim, K. S. Park, S.-W. Park, S. Y. Kim, C. S. Shin, J. Biol. Chem. 2003, 278, 23270.
[152]Y. Kang, X. Kang, H. Yang, H. Liu, X. Yang, Q. Liu, H. Tian, Y. Xue, P. Ren, X. Kuang, Y. Cai, M. Tong, L. Li, W. Fan, Pharmacol. Res. 2022, 175, 106020.
[153]S.-S. Park, Y.-J. Lee, S. Song, B. Kim, H. Kang, S. Oh, E. Kim, J. Endocrinol. 2018, 237, 87.
[154]S. Kim, S.-I. Choi, M. Jang, Y. Jeong, C.-H. Kang, G.-H. Kim, Food Sci. Biotechnol. 2020, 29, 1541.
[155]J.-Y. Kim, E.-J. Park, H.-J. Lee, Int. J. Mol. Sci. 2022, 23, 5901.
[156]L. A. Ribeiro, V. Azevedo, Y. Le Loir, S. C. Oliveira, Y. Dieye, J.-C. Piard, A. Gruss, P. Langella, Appl. Environ. Microbiol. 2002, 68, 910.
[157]H. C. Beck, S. M. Madsen, J. Glenting, J. Petersen, H. Israelsen, M. R. Norrelykke, M. Antonsson, A. M. Hansen, FEMS Microbiol. Lett. 2009, 297, 61.
[158]M. V. Herias, C. Hessle, E. Telemo, T. Midtvedt, L. A. Hanson, A. E. Wold, Clin. Exp. Immunol. 2001, 116, 283.
[159]M. Schultz, C. Veltkamp, L. A. Dieleman, W. B. Grenther, P. B. Wyrick, S. L. Tonkonogy, R. B Sartor, Inflamm. Bowel Dis. 2002, 8, 71.
[160]H. Kinoshita, H. Uchida, Y. Kawai, T. Kawasaki, N. Wakahara, H. Matsuo, M. Watanabe, H. Kitazawa, S. Ohnuma, K. Miura, A. Horii, T. Saito, J. Appl. Microbiol. 2008, 104, 1667.
[161]R. Nandakumar, K. Talapatra, J Dairy Sci. 2014, 97, 1999.
[162]P. Alvarez-Sieiro, M. Montalban-Lopez, D. Mu, O. P. Kuipers, Appl. Microbiol. Biotechnol. 2016, 100, 2939.
[163]M. P. Mokoena, Molecules 2017, 22, 1255.
[164]L. M. T. Dicks, L. Dreyer, C. Smith, A. D. A. R van Staden, Front. Microbiol. 2018, 9, 2938.
[165]J. Zheng, S. Wittouck, E. Salvetti, C. M. A. P. Franz, H. M. B. Harris, P. Mattarelli, P. W. O'toole, B. Pot, P. Vandamme, J. Walter, K. Watanabe, S. Wuyts, G. E. Felis, M. G. Ganzle, S. Lebeer, Int. J. Syst. Evol. Microbiol. 2020, 70, 2782.
[166]M. Kleerebezem, P. Hols, E. Bernard, T. Rolain, M. Zhou, R. J. Siezen, P. A. Bron, FEMS Microbiol. Rev. 2010, 34, 199.
[167]J. L. Ramirez-Zacarias, F. Castro-Munozledo, W. Kuri-Harcuch, Histochemistry 1992, 97, 493.
[168]M. R. Green, J. Sambrook Molecular Cloning A LABORATORY MANUAL, Cold Spring Harbor Laboratory Press, Cold Spring Harbor, New York, 2012.

### ADDITIONAL REFERENCES

[169]M. Sloan & N. Sheth “Projected Volume of Primary and Revision Total Joint Arthroplasty in the United States”, 2030-2060, in Annual Meeting of the American Academy of Orthopedic Surgeons 16, 2018.
[170]A. S. Pugazhendhi et al. “Extracellular Proteins Isolated from L. acidophilus as an Osteomicrobiological Therapeutic Agent to Reduce Pathogenic Biofilm Formation, Regulate Chronic Inflammation, and Augment Bone Formation In Vitro” Advanced Healthcare Materials 13, 2302835, 2024. DOI:10.1002/adhm.202302835
[171] Xu, C. et al. “Cerium oxide nanoparticle: a remarkably versatile rare earth nanomaterial for biological applications”, NPG Asia Mater. 6, (No. 3) (2014).
[172] Celardo, I. et al. “Pharmacological potential of cerium oxide nanoparticles” Nanoscale 3, (No. 4) (2011).
[173] Lord, M. S. et al. Redox Active Cerium Oxide Nanoparticles: Current Status and Burning Issues. Small 2021, 17, 2102342. DOI:10.1002/smll.202102342
[174] Hirst, S. M. et al. “Anti-Inflammatory Properties of Cerium Oxide Nanoparticles” Small 2009 5(24): 2848-2856. DOI:10.1002/smll.200901048
[175] Neal, C. J. et al. “Aging of Nanoscale Cerium Oxide in a Peroxide Environment: Its Influence on the Redox, Surface, and Dispersion Character” J. Phys. Chem. C 2021 125(49):27323-27334. doi.org/10.1021/acs.jpcc.1c06279
[176] Neal, C. J. et al. “Metal-Mediated Nanoscale Cerium Oxide Inactivates Human Coronavirus and Rhinovirus by Surface Disruption” ACS Nano 2021 15(9):14544-14556. doi:10.1021/acsnano.1c04142
[177] Chen, H. et al. “Synthesis of Nanocrystalline Cerium Oxide Particles by the Precipitation Method” Ceramics International 2005 31:795-802. doi:10.10i6/j.ceramint.2004 09.006

1-59. (canceled)

60. A composition comprising cerium oxide nanoparticles and at least one bioactive agent released by or isolated from probiotic organisms.

61. The composition according to claim 60, wherein the probiotic organisms are bacteria.

62. The composition according to claim 61, wherein the bacteria are Lactobacillus acidophilus (L. acidophilus).

63. The composition according to claim 60, wherein the at least one bioactive agent is a protein.

64. The composition according to claim 60, wherein the composition is osteoinductive.

65. A method for decreasing expression of at least one pro-inflammatory gene in cells or tissue, the method comprising administering the composition according to claim 60 to the cells or to the tissue, thereby decreasing expression of the at least one pro-inflammatory gene, wherein the at least one pro-inflammatory gene is RANKL (receptor activator of nuclear factor Kappa-B ligand).

66. A method for increasing expression of at least one anti-inflammatory gene in cells or tissue, the method comprising administering the composition according to claim 60 to the cells or to the tissue, thereby increasing expression of the at least one anti-inflammatory gene, wherein the at least one anti-inflammatory gene is at least one of TGF-β (transforming growth factor beta), IL-10 (interleukin-10), CD-163 (Cluster of Differentiation-163), and CD-206 (Cluster of Differentiation-206).

67. A pharmaceutical composition comprising cerium oxide nanoparticles coated with at least one extracellular protein isolated from Lactobacillus acidophilus (L. acidophilus).

68. The pharmaceutical composition according to claim 67, wherein surfaces of the cerium oxide nanoparticles are partially or completely coated with the at least one extracellular protein isolated from Lactobacillus acidophilus (L. acidophilus).

69. The pharmaceutical composition according to claim 67, wherein the pharmaceutical composition is a therapeutic solution, gel, paste, or topical cream.

70. The pharmaceutical composition according to claim 68, wherein the pharmaceutical composition is an additive formulated for another composition.

71. An implant configured for insertion into a body, the implant comprising a surface coated with a pharmaceutical composition, wherein the pharmaceutical composition comprises cerium oxide nanoparticles coated with at least one extracellular protein isolated from Lactobacillus acidophilus (L. acidophilus).

72. A kit comprising the implant according to claim 71.

73. A method for treating a condition in a subject in need thereof, the method comprising:
providing the implant according to claim 71; and
inserting the implant into the subject, thereby treating the condition.

74. The method according to claim 73, wherein the subject is a human and the condition is a periprosthetic joint infection (PJI).

75. A method for healing or repairing injured tissues by stimulating new bone formation in injured tissue, the method comprising:
providing the implant according to claim 71; and
inserting the implant into the injured tissue, thereby stimulating new bone formation in the injured tissue to heal or repair the injured tissue.

76. A method for protecting cells and tissue from radiation exposure, the method comprising:
providing the implant according to claim 71; and
inserting the implant into the subject, thereby protecting the cells and tissue from radiation exposure.

77. A method for protecting cells and tissue from effects of aging, the method comprising:
providing the implant according to claim 71; and
inserting the implant into the subject, thereby protecting the cells and tissue from the effects of aging.
